(19) United States
(12) Patent Application Publication
(10) Pub. No.: US 2004/0030367 A1
(43) Pub. Date: Feb. 12, 2004

(54) Medical control device, control method for medical control device, medical system device and control system

(75) Inventors: Masahide Yamaki, Tokyo (JP); Kenji Noda, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS OPTICAL CO., LTD., TOKYO (JP)

(21) Appl. No.: 10/635,045

(22) Filed: Aug. 5, 2003

(30) Foreign Application Priority Data
Aug. 9, 2002 (JP) 2002-233670
Oct. 3, 2002 (JP) 2002-291564
Oct. 3, 2002 (JP) 2002-291565
Nov. 8, 2002 (JP) 2002-325817

(51) Int. Cl. A61N 1/08

(52) U.S. Cl. 607/60

(57) ABSTRACT

The system controller comprises a character superimposing unit which superimposes desired characters on an endoscopic image and outputs the image, a setting operating unit I/F unit which transmits and receives data to and from an operating panel, a unidirectional infrared I/F unit which performs infrared communications with an infrared remote controller, a bidirectional infrared I/F unit which performs infrared communications with a PDA, a remote controller control I/F unit which transmits and receives data to and from a remote controller, and a serial communications I/F unit which performs serial communications, and is constructed so that these parts are connected to an internal bus. A CPU which controls the interior of the system controller is connected to the internal bus. Even if communications are performed with a plurality of devices that have different communications formats, the control of the plurality of devices with different communications formats can be quickly performed without increasing the cost or increasing the size of the apparatus.

This application claims benefit of Japanese Application Nos. 2002-233670 filed on Aug. 9, 2002, 2002-291564 filed on Oct. 3, 2002, 2002-291565 filed on Oct. 3, 2002, and 2002-325817 filed on Nov. 8, 2002, the contents of which are incorporated by this reference.

### BACKGROUND OF THE INVENTION

1. Field of the Invention

The present invention relates to a medical control device, control method for a medical control device, medical system device and control system which control medical devices that are used to perform medical treatments.

2. Description of the Related Art

In recent years, a more compact size and a higher degree of functions have been required in computers. For example, low power consumption type personal computers and the like have been developed in order to improve the portability of devices. Furthermore, ether net communications that transmit large quantities of data at a high speed, compact portable terminals known as portable information terminals (personal digital assistance; hereafter referred to as “PDA”), such a palm top computers and the like, and infrared communications (infrared data association; hereafter referred to as “IrDA”) that transmit data, are available as means of improving the expandability of systems.

Meanwhile, a system control device (hereafter referred to as a “system controller”) in which the functions of a plurality of medical devices are displayed on a menu screen and these medical devices are controlled by operating this displayed menu screen in order to improve operability as a surgical system is disclosed in Japanese Patent Application Laid-Open No. 7-303654.

A medical device in which information from an external device is input, and this input information is displayed by a display device together with an observation image, is disclosed in Japanese Patent Application Laid-Open No. 11-318823.

It is conceivable that ether net communications, IrDA communications or the like that allow high-speed data communications might be used for the input of such external information.

Furthermore, endoscopic surgical systems exist as one type of surgical system equipped with a plurality of devices used for surgical procedures and a system controller that performs comprehensive control of these surgical devices. A common endoscopic surgical system comprises an endoscope that is used for observation, a camera head that is connected to the endoscope, a light source device that provides illuminating light to the observation site via the abovementioned endoscope, an endoscopic camera device that processes image signals acquired by the abovementioned camera head, a monitor that displays object images of the observation site processed by the endoscopic camera device, an insufflator that is used to expand the abdominal cavity, and a plurality of medical devices that are surgical devices used to perform surgical procedures, such as a high-frequency cauterizing device that excises biological tissues or causes coagulation. These surgical devices are mainly operated and used by surgeons.

Meanwhile, in the operating room, there are also devices known as patient monitoring devices, which are monitored mainly by anesthesiologists. Such devices are devised so that biological information relating to the patient (hereafter referred to as “vital signs”) can be monitored in a concentrated manner. An electrocardiograph, pulse oximeter, capnometer and the like are connected to this device, so that vital signs such as electrocardiograms, concentration of carbon dioxide gas in the breath, blood pressure, degree of oxygen saturation in the blood and the like can be measured and displayed in a concentrated display.

Furthermore, hospital systems in which patient monitoring devices in respective operating rooms, camera controller units (hereafter referred to as “CCU”) and patient monitoring devices installed in hospital rooms are connected by communications means, and monitoring and recording are performed in doctors' offices or nurse stations, have been proposed.

A system control device in which means for displaying the functions of controlled devices and means for operating the controlled devices are provided in order to allow easy operation and control of a plurality of surgical devices so that the operability as a system is improved is disclosed in Japanese Patent Application Laid-Open No. 7-303654.

Furthermore, a medical device which has means for inputting signals from external communications devices and display means for displaying information as medical treatment information on the basis of the input signals, and which can display medical treatment information from locations other than the operating room together with endoscopic images, is disclosed in Japanese Patent Application Laid-Open No. 11-318823. Furthermore, an anesthetic device which measures biological information for the patient is disclosed as one example of an external device, and a method for connecting surgical devices and biological information for the patient by communications is disclosed.

Furthermore, a medical system which is characterized in that this system comprises a plurality of surgical systems and communications means for connecting these systems and transmitting and receiving data, so that shared data can be acquired by the respective systems, is proposed in Japanese Patent Application Laid-Open No. 2001-000449.

In the conventional medical device systems described above, the following problem has been encountered: specifically, since there is no association between the surgical devices and the patient monitoring devices, respective communications means are required in the hospital system, so that the devices and systems become complicated and bothersome.

Furthermore, the following problem has also been encountered: specifically, in cases where no anesthesiologist is present during the operation, the surgeon may not be able to make a determination in response to emergencies when abnormalities occur in the vital signs of the patient, so that time must be taken to find an anesthesiologist.

Accordingly, for example, a medical device communications system which can provide information that immediately allows the surgeon to make an appropriate determination in cases where abnormalities occur in the vital signs of the patient has been proposed in Japanese Patent Application Laid-Open No. 2002-065618 and the like.

Furthermore, for example, endoscopic systems used for medical treatment which are equipped with an endoscope may be cited as examples of systems comprising a plurality of devices. In the case of common endoscopic systems, the system comprises an endoscope that is used for observation, a camera head that is connected to the endoscope, an endoscopic camera device that processes the image signals acquired by the camera head, a light source device that supplies illuminating light to the object of observation, a monitor that displays an image of the object of observation and the like. This system is devised so that the endoscope is inserted to the observation site, the object of observation is illuminated by illuminating light from the light source device so that an optical image of the object of observation is obtained by the endoscope, the image signal of the object image obtained by the camera head is subjected to signal processing by the endoscopic camera device, and an image of the object of observation is displayed on the monitor. Observation and examinations inside body cavities and the like can be performed using such an endoscopic system.

In recent years, surgical techniques and the like using endoscopes have also been performed. In the case of such endoscopic surgical techniques, an insufflator which is used to expand the abdominal cavity, a high-frequency cauterizing device used to excise biological tissues (which is a treatment device used to perform surgical procedures) and the like are used as surgical devices in addition to the devices described above, and various types of procedures are performed while the treatment site is observed via the endoscope, as indicated (for example) in the abovementioned Japanese Patent Application Laid-Open No. 7-303654.

### SUMMARY OF THE INVENTION

The medical control device of the present invention comprises a first communications control unit which utilizes communications of a first protocol to transmit and receive data to and from a first medical device that is used to perform medical treatments, a second communications control unit which utilizes communications of a second protocol that differs from the abovementioned first protocol to transmit and receive data to and from a second medial device that is used to perform medical treatments, and a control part which transmits and receives data utilizing communications of a third protocol that is common to the above-mentioned first communication control unit and the abovementioned second communications control unit, and which controls the abovementioned first communications control unit and the abovementioned second communications control unit.

Objects, features and advantages of the invention will become more clearly understood from the following description referring to the accompanying drawings.

### BRIEF DESCRIPTION OF THE DRAWINGS

FIG. 1 is a structural diagram which shows the construction of an endoscopic surgical system constituting a first embodiment of the present invention;

FIG. 2 is a structural diagram which shows the construction of a patient monitoring system that monitors the status of the patient in FIG. 1;

FIG. 3 is a diagram which shows a hospital network in which the endoscopic surgical system shown in FIG. 1 is disposed;

FIG. 4 is a diagram which shows one example of an internet connection service to which the hospital server shown in FIG. 3 is connected;

FIG. 5 is a diagram which shows the front-view construction of the system controller 22 shown in FIG. 1;

FIG. 6 is a diagram which shows the back-view construction of the system controller 22 shown in FIG. 1;

FIG. 7 is a block diagram which shows the construction of the system controller shown in FIG. 1;

FIG. 8 is a block diagram which shows the construction of the PDA shown in FIG. 1;

FIG. 9 is a diagram which shows the operating part of the operating panel shown in FIG. 1;

FIG. 10 is a diagram which shows the front-view construction of the PDA shown in FIG. 1;

FIG. 11 is a diagram which shows the back-view construction of the PDA shown in FIG. 1;

FIG. 12 is a diagram which is used to illustrate the expansion card mounted in the card slot shown in FIG. 11;

FIG. 13 is a diagram which illustrates the infrared remote controller shown in FIG. 1;

FIG. 14 is a flow chart which shows the flow of the processing of the CPU of the system controller in a second embodiment of the present invention;

FIG. 15 is a diagram which illustrates the main processing in FIG. 14;

FIG. 16 is a flow chart which shows the flow of the task handling processing in FIG. 15;

FIG. 17 is a diagram which illustrates the fixed-period processing part in FIG. 14;

FIG. 18 is a diagram which illustrates the part that performs communications processing according to function in FIG. 14;

FIG. 19 is a flow chart which shows the flow of system initialization processing in FIG. 14;

FIG. 20 is a flow chart which shows the flow of the communications port checking processing of the fixed-period processing part shown in FIG. 17;

FIG. 21 is a flow chart which shows the flow of the character superimposition processing of the fixed-period processing part shown in FIG. 17;

FIG. 22 is a flow chart which shows the flow of the peripheral device communications processing of the part that performs communications processing according to function shown in FIG. 18;

FIG. 23 is a flow chart which shows the data write processing in FIG. 22;

FIG. 24 is a flow chart which shows the data read-in processing in FIG. 22;

FIG. 25 is a flow chart which shows the flow of the setting display communications processing of the part that performs communications according to function shown in FIG. 18;

FIG. 26 is a flow chart which shows the flow of the PDA communications processing of the part that performs communications according to function shown in FIG. 18;

FIG. 27 is a flow chart which shows the flow of the remote controller communications processing of the part that performs communications according to function shown in FIG. 18;

FIG. 28 is a flow chart which shows the flow of the anesthetic device communications processing of the part that performs communications according to function shown in FIG. 18;

FIG. 29 is a first time chart which is used to illustrate the flow charts of FIGS. 15 and 16;

FIG. 30 is a second time chart which is used to illustrate the flow charts of FIGS. 15 and 16;

FIG. 31 is a third time chart which is used to illustrate the flow charts of FIGS. 15 and 16;

FIG. 32 is a fourth time chart which is used to illustrate the flow charts of FIGS. 15 and 16;

FIG. 33 is a block diagram which shows the construction of the system controller of an endoscopic surgical system constituting a third embodiment of the present invention;

FIG. 34 is a diagram which shows the construction of the remote controller in the third embodiment;

FIG. 35 is a diagram which shows the display screen of a display device that displays endoscopic images;

FIG. 36 is a diagram which is used to illustrate the pattern of the superimposed data that is superimposed on the display screen shown in FIG. 35;

FIG. 37 is a flow chart which illustrates the operation of the system controller;

FIG. 38 is a flow chart which shows the flow of the ON processing of the superimposed display in FIG. 37;

FIG. 39 is a structural diagram which shows the construction of the remote controller in a fourth embodiment of the present invention;

FIG. 40 is a diagram which shows the superimposed data setting screen that is set and operated by the remote controller shown in FIG. 39;

FIG. 41 is a block diagram which shows the construction of the essential parts of the operating panel in a fifth embodiment of the present invention;

FIG. 42 is a diagram which shows the connection relationship between the system controller and operating panel in the fifth embodiment of the present invention;

FIG. 43 is a first flow chart which illustrates the operation of the endoscopic surgical system of the fifth embodiment of the present invention;

FIG. 44 is a second flow chart which illustrates the operation of the endoscopic surgical system of the fifth embodiment of the present invention;

FIG. 45 is a third flow chart which illustrates the operation of the endoscopic surgical system of the fifth embodiment of the present invention;

FIG. 46 is a fourth flow chart which illustrates the operation of the endoscopic surgical system of the fifth embodiment of the present invention;

FIG. 47 is a block diagram which shows the construction of the infrared remote controller in a sixth embodiment of the present invention;

FIG. 48 is a flow chart which shows the flow of the processing that is performed when a peripheral device is operated by unidirectional infrared remote controller in the sixth embodiment of the present invention;

FIG. 49 is a block diagram which shows the construction of the touch panel and wireless communications interface (I/F) in the sixth embodiment of the present invention;

FIG. 50 is a diagram which shows a second screen displayed by the liquid crystal display part in the sixth embodiment;

FIG. 51 is a diagram which shows a third screen displayed by the liquid crystal display part in the sixth embodiment;

FIG. 52 is a diagram which shows a fourth screen displayed by the liquid crystal display part in the sixth embodiment;

FIG. 53 is a diagram which shows a fifth screen displayed by the liquid crystal display part in the sixth embodiment;

FIG. 54 is a diagram which shows a sixth screen displayed by the liquid crystal display part in the sixth embodiment;

FIG. 55 is a diagram which shows a seventh screen displayed by the liquid crystal display part in the sixth embodiment;

FIG. 56 is a diagram which shows an eighth screen displayed by the liquid crystal display part in the sixth embodiment;

FIG. 57 is a diagram which shows a ninth screen displayed by the liquid crystal display part in the sixth embodiment;

FIG. 58 is a diagram which shows a tenth screen displayed by the liquid crystal display part in the sixth embodiment;

FIG. 59 is a diagram which shows an eleventh screen displayed by the liquid crystal display part in the sixth embodiment;

FIG. 60 is a diagram which shows a twelfth screen displayed by the liquid crystal display part in the sixth embodiment;

FIG. 61 is a diagram which shows the construction of the unidirectional infrared communications controller of the unidirectional infrared communications interface (I/F) in the sixth embodiment;

FIG. 62 is a diagram which shows the construction of the bidirectional infrared communications controller of the bidirectional infrared communications interface (I/F) in the sixth embodiment;

FIG. 63 is a first flow chart which shows the flow of the processing that is performed when a peripheral device is operated by the PDA in the sixth embodiment;

FIG. 64 is a second flow chart which shows the flow of the processing that is performed when a peripheral device is operated by the PDA in the sixth embodiment;

FIG. 65 is a flow chart which illustrates the operation of the unidirectional infrared communications controller and bidirectional infrared communications controller shown in FIG. 61 and FIG. 62;

FIG. 66 is a diagram which is used to describe the flow chart shown in FIG. 65;

FIG. 67 is a block diagram which shows the essential parts of the construction of the PDA in a seventh embodiment of the present invention;

FIG. 68 is a flow chart which shows the flow of the processing that is performed when the PDA of an eighth embodiment of the present invention is operated;

FIG. 69 is a diagram which shows the state of the display part of the first PDA in a ninth embodiment of the present invention;

FIG. 70 is a diagram which shows the state of the display part of the second PDA in the ninth embodiment of the present invention; and

FIG. 71 is a flow chart which illustrates the software operation that transmits communications limiting commands from the system controller to a specified PDA in the ninth embodiment of the present invention.

### DETAILED DESCRIPTION OF THE PREFERRED EMBODIMENTS

Embodiments of the present invention will be described below with reference to the attached figures.

[First Embodiment]

[Construction]

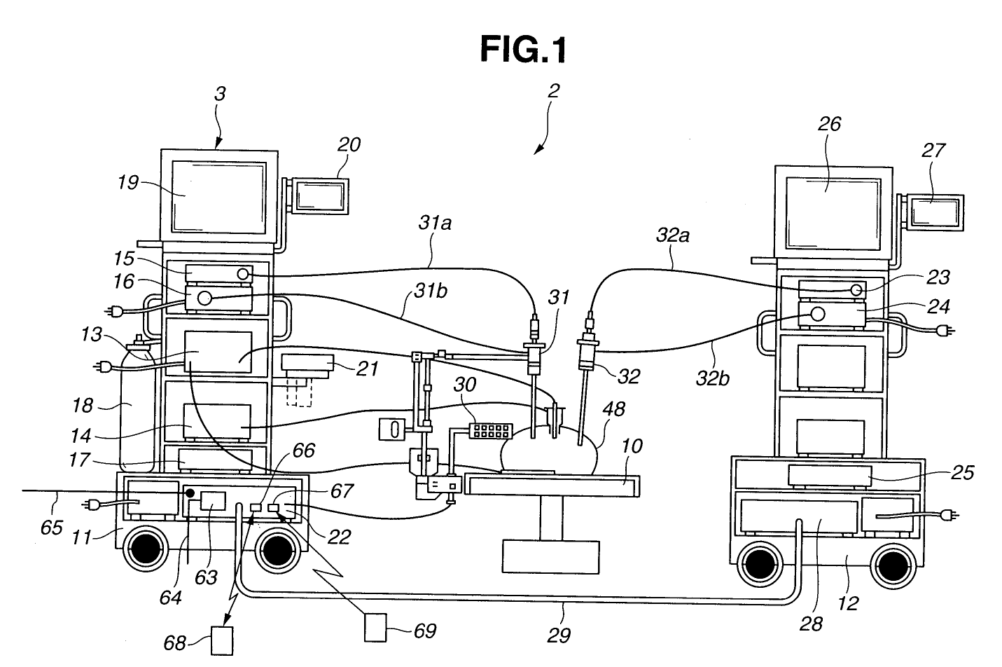

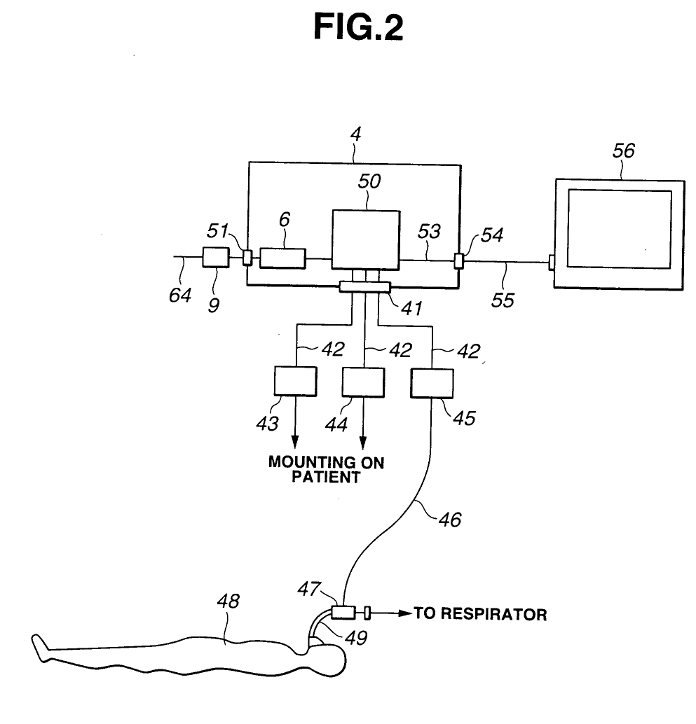

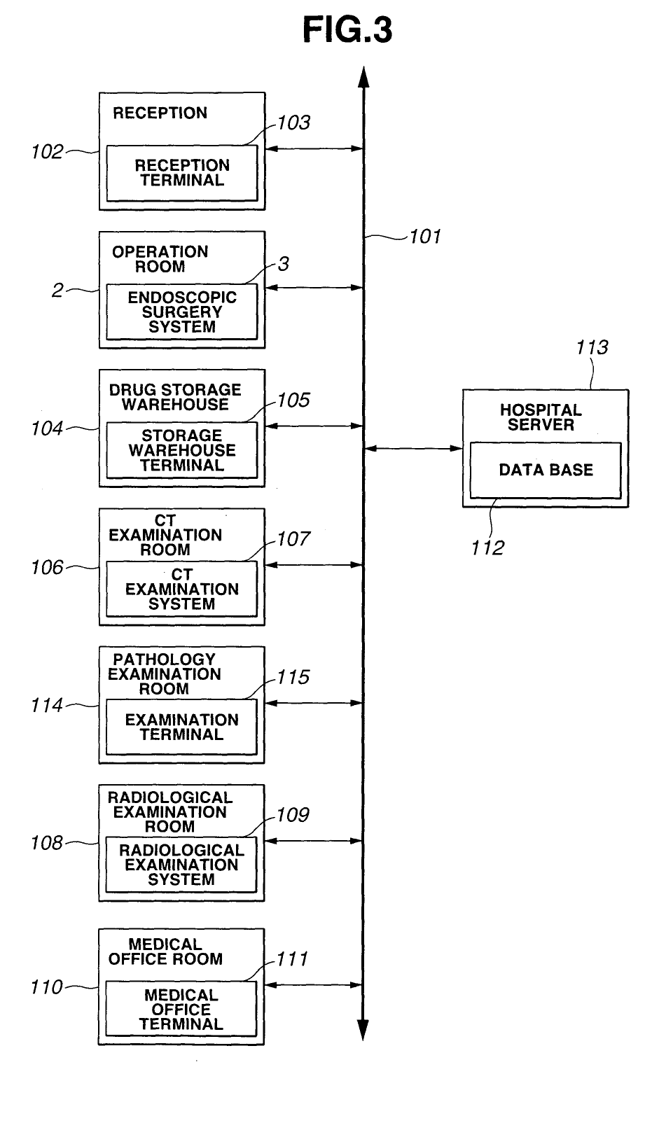

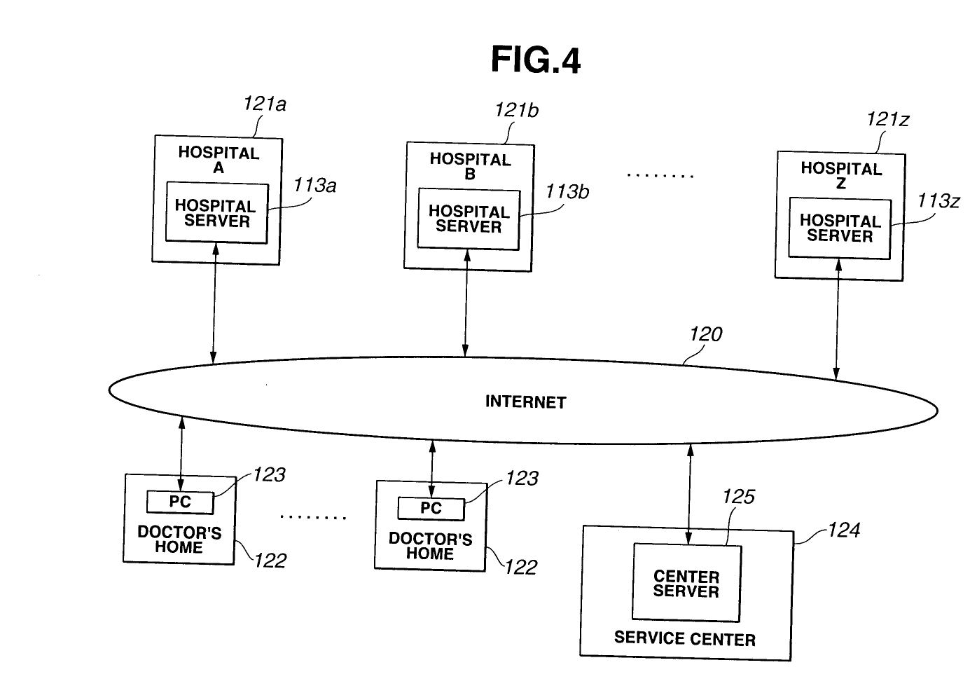

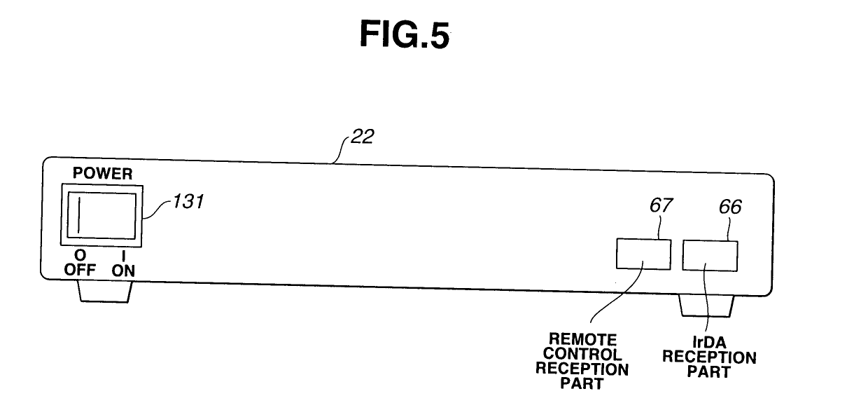

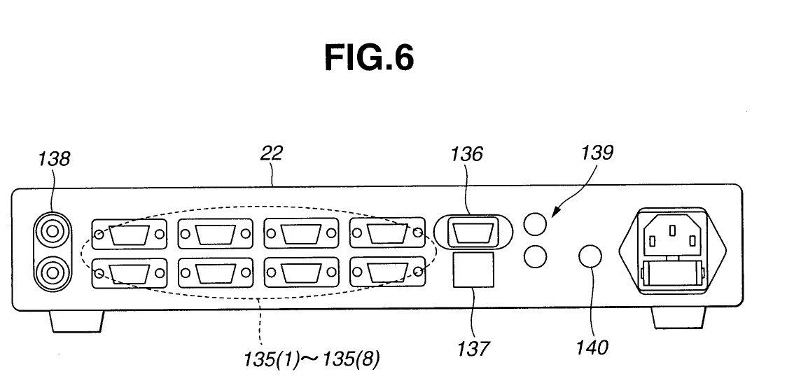

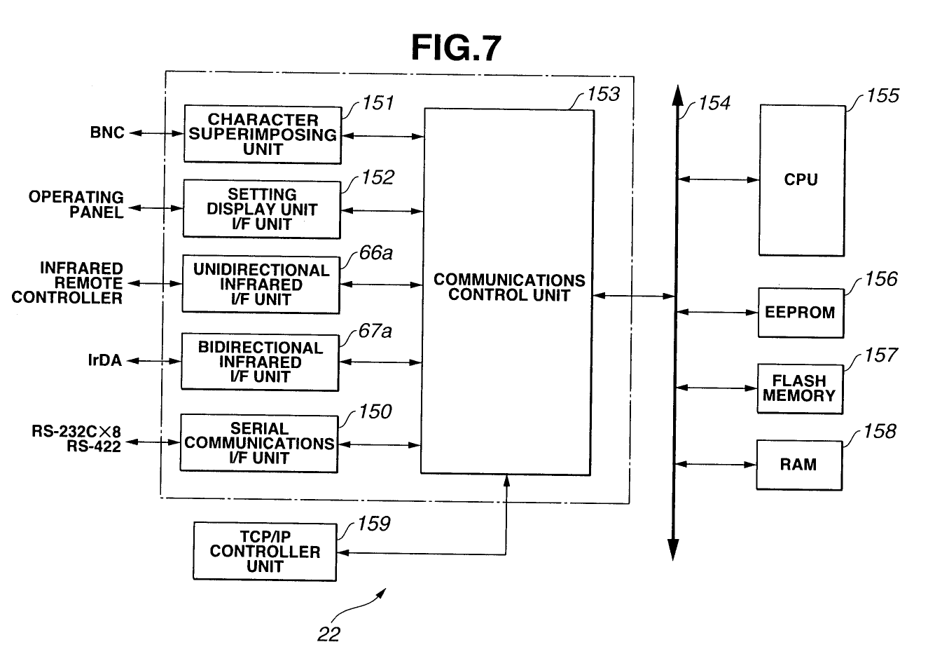

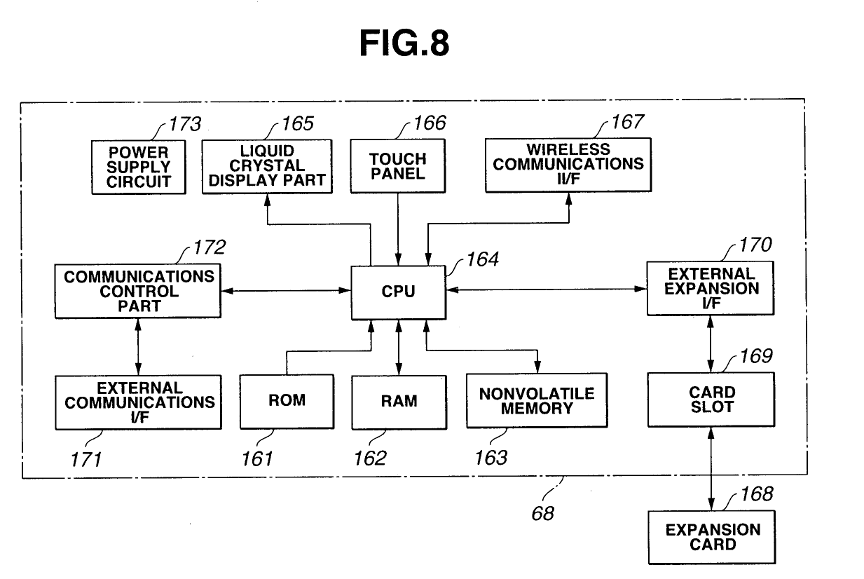

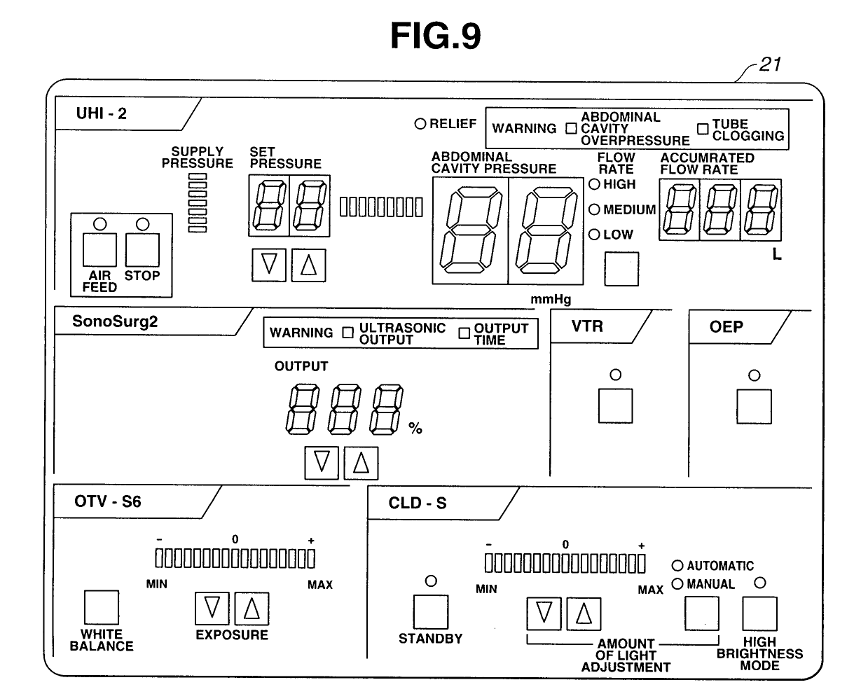

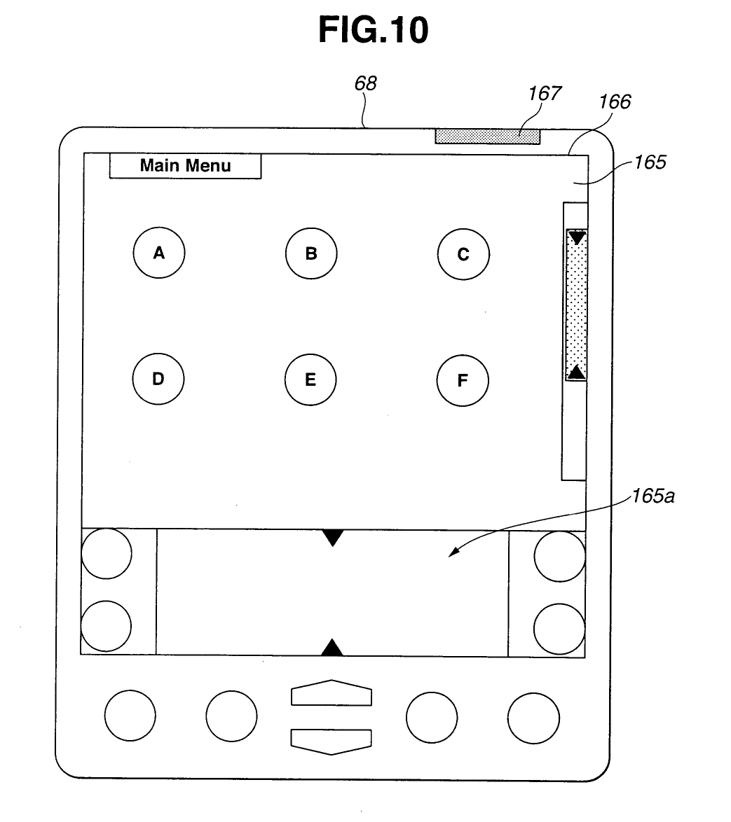

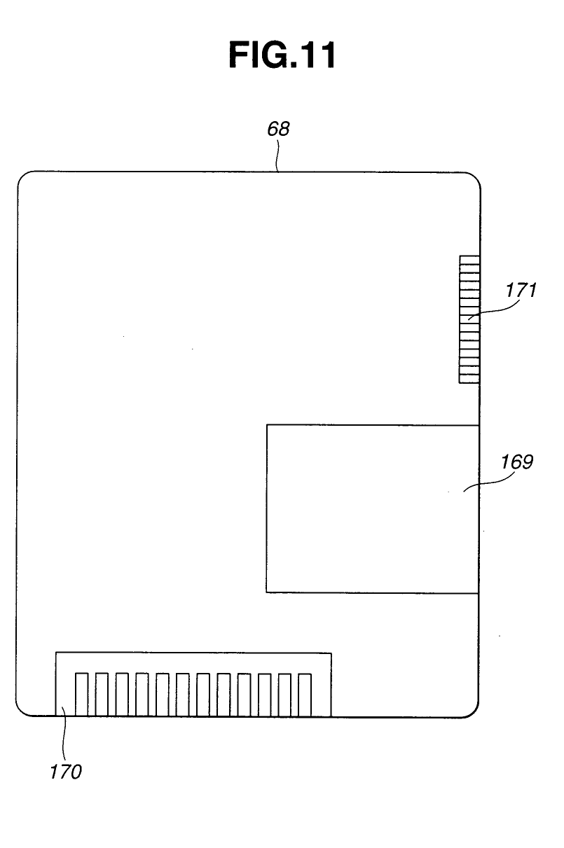

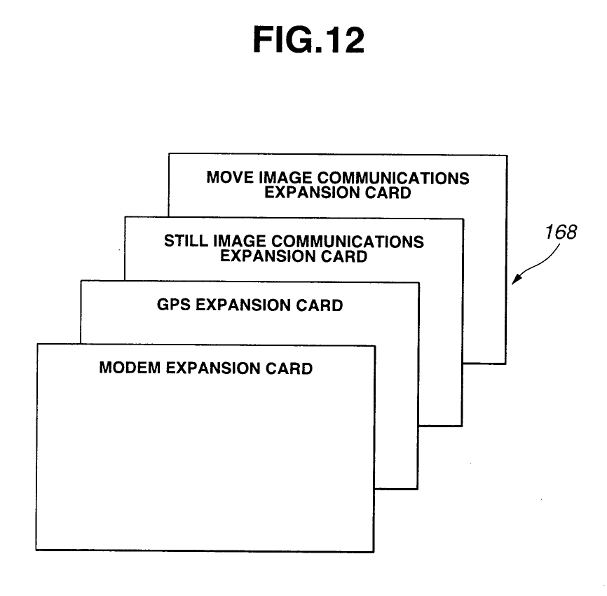

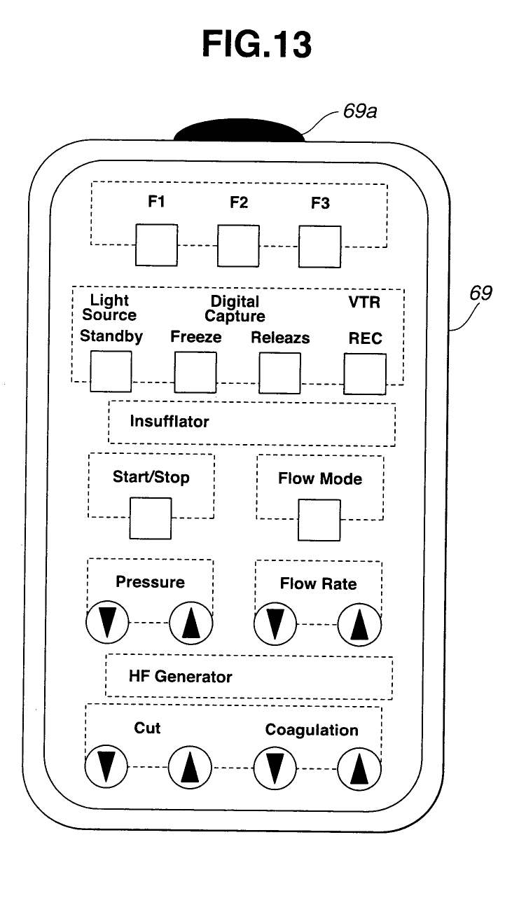

FIGS. 1 through 13 relate to a first embodiment of the present invention. FIG. 1 is a structural diagram which shows the construction of an endoscopic surgical system, FIG. 2 is a structural diagram which shows the construction of a patient monitoring system that monitors the status of the patient in FIG. 1, FIG. 3 is a diagram which shows a hospital network in which the endoscopic surgical system shown in FIG. 1 is disposed, FIG. 4 is a diagram which shows one example of an internet connection service to which the hospital server shown in FIG. 3 is connected, FIG. 5 is a diagram which shows the front-view construction of the system controller 22 shown in FIG. 1, FIG. 6 is a diagram which shows the back-view construction of the system controller 22 shown in FIG. 1, FIG. 7 is a block diagram which shows the construction of the system controller shown in FIG. 1, FIG. 8 is a block diagram which shows the construction of the PDA shown in FIG. 1, FIG. 9 is a diagram which shows the operating part of the operating panel and the display unit shown in FIG. 1, FIG. 10 is a diagram which shows the front-view construction of the PDA shown in FIG. 1, FIG. 11 is a diagram which shows the back-view construction of the PDA shown in FIG. 1, FIG. 12 is a diagram which is used to illustrate the expansion card mounted in the card slot shown in FIG. 11, and FIG. 13 is a diagram which illustrates the infrared remote controller shown in FIG. 1.

The overall construction of the endoscopic surgical system 3 which is disposed in an operating room 2 will be described with reference to FIG. 1.

As is shown in FIG. 1, a patient bed 10 on which the patient 48 lies, and the endoscopic surgical system 3, are disposed inside the operating room 2. The endoscopic surgical system 3 has a first cart 11 and a second cart 12.

For example, devices such as an electrical scalpel 13, an insufflator 14, an endoscopic camera device (CCU) 15, a light source device 16, a VTR 17 and the like, and a gas cylinder 18 filled with carbon dioxide gas or the like, are disposed on the first cart 11 as medical devices. The endoscopic camera device 15 is connected to a first endoscope 31 via a camera cable 31a. The light source device 16 is connected to the first endoscope 31 via a light guide cable 31b.

Furthermore, a display device 19, a concentrated display panel 20, an operating panel 21 and the like are disposed on the first cart 11. For example, the display device 19 is a TV monitor that displays endoscopic images and the like.

The concentrated display panel 20 constitutes display means that allow the selective display of all types of data during the operation. The operating panel 21 is a concentrated operating device which comprises, for example, display parts such as a seven-segment display device, LEDs and the like, and switches that are mounted on these display parts, and which is operated by a nurse or the like in a non-sterile area.

Furthermore, a system controller 22 is disposed on the first cart 11. The abovementioned electrical scalpel 13, insufflator 14, endoscopic camera device 15, light source device 16 and VTR 17 are connected to the system controller 22 via communications lines (not shown in the figures) in accordance with serial communications standards such as RS-232C or the like. A communications controller 63 is disposed inside the system controller 22, and this is connected to the communications circuit 9 shown in FIG. 2 via a communications cable 64. Furthermore, the system controller 22 is connected to a hospital LAN via a communications cable 65. Moreover, a bidirectional infrared communications interface (hereafter abbreviated to “I/F”) 66 and a unidirectional infrared communications I/F 67 are disposed in the system controller 22, so that the transmission and reception of signals to and from a PDA 68 (see FIGS. 10 and 11) can be accomplished by IrDA communications via the bidirectional infrared communications I/F 66, and so that the reception of commands by infrared communications from an infrared remote controller 69 (see FIG. 13) can be accomplished via the unidirectional infrared communications I/F 67. Furthermore, the PDA 68 can also be connected to the system controller by serial communications.

Meanwhile, an endoscopic camera device 23, light source device 24, image processing device 25, display device 26 and second concentrated display panel 27 are disposed on the abovementioned second cart 12.

The endoscopic camera device 23 is connected to a second endoscope 32 via a camera cable 32a. The light source device 24 is connected to the second endoscope 32 via a light guide cable 32b.

The display device 26 displays endoscopic images and the like acquired by the endoscopic camera device 23. The second concentrated display panel 27 can selectively display all types of data during the operation.

The abovementioned endoscopic camera device 23, light source device 24 and image processing device 25 are connected via communications lines (not shown in the figures) to a relay unit 28 disposed on the second cart 12. Furthermore, the relay unit 28 is connected to the system controller 22 disposed on the abovementioned first cart 11 via a relay cable 29.

Accordingly, the system controller 22 can perform concentrated control of the camera device 23, light source device 24 and image processing device 25 disposed on the second cart 12, and the electrical scalpel 13, insufflator 14, camera device 15, light source device 16 and VTR 17 disposed on the first cart 11. Consequently, in cases where communications are established between the system controller 22 and these devices, the system controller 22 can display the set states of the connected devices and the setting screens of the operating switches and the like on the liquid crystal display of the abovementioned operating panel 21 for purposes of confirmation; furthermore, operating input such as the alteration of setting values and the like can be accomplished by touching desired operating switches and operating touch sensors in predetermined regions.

The system controller 22 can analyze biological information acquired from a patient monitoring system 4 (described later), and can display the results of this analysis on a predetermined display device.

Next, the patient monitoring system 4 will be described with reference to FIG. 2.

As is shown in FIG. 2, a signal connection part 41 is disposed in the patient monitoring system 4 of the present embodiment. The signal connection part 41 is connected to vital sign measuring devices such as an electrocardiogram 43, pulse oximeter 44, capnometer 45 and the like via cables 42.

The capnometer 45 is connected to a breath sensor 47 via a cable 46. The breath sensor 47 is installed in the hose 49 of a respirator that is attached to the patient 48. As a result, biological information such as an electrocardiogram, degree of saturation of oxygen in the blood, concentration of carbon dioxide gas in the breath and the like can be measured for the patient 48.

The signal connection part 41 is electrically connected to a control part 50 inside the patient monitoring system 4. Furthermore, the control part 50 is connected to a display device 56 via a video signal line 53, video connector part 54 and cable 55. Moreover, the control part 50 is electrically connected to a communications controller 6. The communications controller 6 is connected to a communications circuit 9 via a communications connector 51.

The communications circuit 9 is connected to the communications controller (not shown in the figures) of the abovementioned endoscopic surgical system 3.

As is shown in FIG. 3, the endoscopic surgical system 3 installed in the operating room 2 is connected to a hospital LAN 101 which is constructed inside the hospital via the system controller 22.

A reception terminal 103 which is disposed in another facility of the hospital, e.g., reception 102, a storage warehouse terminal 105 which is disposed in a drug storage warehouse 104, a CT examination system (system controller) 107 which is disposed in a CT examination room 106, a radiological examination system (system controller) 109 which is disposed in a radiological examination room 108, a medical office terminal 111 which is disposed in a medical office 110, and a pathology terminal 115 which is disposed in a pathology examination room 114, are connected to the hospital LAN 101, and the hospital LAN 101 is controlled by a hospital server 113 that constructs a data base 112.

Furthermore, as is shown in FIG. 4, the hospital server 113 is connected to the internet 120, and personal computers (hereafter referred to as “PCs”) 123 installed in doctors' homes 122, as well as hospital servers 113a through 113z of a plurality of hospitals 121a through 121z, are connected to the internet 120, so that (for example) the center server 125 of a service center 124 can operate a service that provides medical information to hospitals and doctors' homes.

As is shown in FIG. 5, a power supply switch 131, the abovementioned unidirectional infrared I/F 67 for the infrared remote controller 69, and the abovementioned bidirectional infrared I/F 66 for the PDA 68, are installed on the front surface of the system controller 22, and as is shown in FIG. 6, eight RS-232C communications connectors 135(1) through 135(8) (for example) which are used to control the electrical scalpel 13, insufflator 14, endoscopic camera device 15, light source device 16, VTR 17, concentrated display panel 20, remote controller 30 and the like, an RS-422 communications connector 136 which is used to control the operating panel 21, a 100T/Base connector 137 which is used for connection with the hospital LAN 101, a BNC 138 which connects the display device 19, a pin jack 139 which transmits and receives video signals to and from the VTR 17, a communications connector 140 which is used for setting control of the operating panel 21 and the like are installed on the back surface of the system controller 22.

As is shown in FIG. 7, the system controller 22 comprises a character superimposing unit 151 which superimposes desired characters on the endoscopic image and outputs this image to the BNC 138, a setting operating unit I/F unit 152 which transmits and receives data to and from the operating panel 21, a unidirectional infrared I/F unit 67a which performs infrared communications with the infrared remote controller 69, a bidirectional infrared I/F unit 66a which performs infrared communications with the PDA 68, and a serial communications I/F unit 150 which is constructed from an FPGA (field programmable gate array) that performs serial communications via the RS-232C communications connectors 135(1) through 135(8) and RS-422 communications connector 136, and is constructed so that these parts are connected to an internal bus 154.

Furthermore, in the present construction, the controller parts (parts indicated by one-dot chain lines in FIG. 7) of a plurality of communications systems, i.e., the abovementioned character superimposing unit 151, setting display unit I/F unit 152 and the like, are constructed using an FPGA, so that the circuit construction is integrated; however, it would also be possible to construct these controller parts using respective independent circuits.

A CPU (central processing unit) 155 which controls the interior of the system control unit 22, and EEPROM (electrically erasable programmable read-only memory) 156, a flash memory 157 used for version upgrading, and a RAM (random-access memory) 158, are connected to the abovementioned internal bus 154, and the CPU 155 controls the interior of the system controller 22 using the EEPROM 156, version upgrading flash memory 157, RAM 158 and the like. Furthermore, setting information for task priority information (described later) and programs executed by the CPU 155 are stored in the EEPROM 156.

Furthermore, a TCP/IP controller unit 159 is connected to the CPU 155 via the FPGA. A connection to the hospital LAN 101 is made via the TCP/IP controller unit 159.

In the present embodiment, the system controller 22 has a character superimposing unit 151, setting display unit I/F 152, unidirectional infrared I/F 67a, bidirectional infrared I/F 66a and serial communications I/F 150 constructed by the abovementioned FPGA. These respective I/Fs are comprised of a driver and a controller for each protocol.

Furthermore, a communications control unit 153 which performs control of the respective I/Fs and exchange of data with the CPU 155 is disposed inside the FPGA, so that a construction is formed in which signals are transferred as bus signals to the CPU 155. The FPGA is connected to the CPU 155 by a signal bus 154 (constructed from a data bus, address bus and select signal). Furthermore, in the present embodiment, the TCP/IP controller unit 159 is independently installed outside the FPGA; however, it would also be possible to install this part inside the FPGA.

As is shown in FIG. 8, the PDA 68 comprises a CPU 164 which controls the interior of the PDA 68 using a ROM (read-only memory) 161, RAM 162, nonvolatile memory 163 and the like, a liquid crystal display unit 165 which displays the information form the CPU 164, a touch panel 166 which is installed on the liquid crystal display part 165 that inputs information into the CPU 164, a wireless communications I/F 167 for bidirectional infrared communications by means of IrDa, Bluetooth, wireless LAN or the like, an external expansion I/F 170 which connects an expansion card 168 for realizing the expansion of functions to the CPU 164 via a card slot 169, a communications control unit 172 which controls communications with external devices connected to an external communications I/F 171, and a power supply circuit 173 which supplies power to these circuits.

As is shown in FIG. 9, the operating panel 21 is comprised of a display function, e.g., a plurality of seven-segment display devices and LEDs or the like, and switches, and is a concentrated operating device which is operated by a nurse or the like in a non-sterile area.

As is shown in FIG. 10, a liquid crystal display part 165 on which a touch panel 166 is installed in disposed on the front surface of the PDA 68, and a portion of the liquid crystal display part 165 constitutes a handwritten input part. Furthermore, as is shown in FIG. 11, a card slot 169 and an external communications I/F 171 are disposed on the back surface of the PDA 68. Examples of expansion cards 168 that can be mounted in the card slot 169 include movie image communications expansion cards, still image communications expansion cards, GPS (global positioning system) expansion cards, modem expansion cards and the like such as those shown in FIG. 12.

Furthermore, as is shown in FIG. 13, various types of operating buttons are disposed on the front surface of the remote controller 69, and infrared command signals are output from an output window part 69a in accordance with the buttons that are operated.

[Effect]

The operation of the first embodiment will be described.

As was described above, a character superimposing unit 151, a setting operating unit I/F unit 152, a unidirectional infrared I/F unit 67a, a bidirectional infrared I/F unit 66a and a serial communications I/F unit 150 comprises of an FPGA are disposed in the system controller 22 of the first embodiment, and the CPU 155 of the system controller 22 designates desired I/F parts by outputting (for example) address data to the internal bus 154, and outputs data to these I/F parts. In the I/F parts that have received data from the CPU 155, the data is converted into a predetermined protocol, and data is exchanged with the peripheral devices connected to these I/F parts. Furthermore, data received from peripheral devices in a predetermined protocol is converted into data that corresponds to the internal bus 154, and is output to the internal bus 154 in accordance with requests from the CPU 155.

For example, in the processing that displays vital signs on the display panel 20, the TCP/IP controller unit 159 receives vital sign information in the TCP/IP protocol, performs data analysis, and outputs the results of the analysis to the communications control unit 153.

The communications control unit 153 recognizes that the received information is vital sign information as a result of the input results of analysis. Then, the communications control unit 153 temporarily stores the protocol-analyzed vital sign information in an internal memory, and confirms the communications operating states of other I/F parts. When the communications operating state of the other I/F parts in this case are states that allow transmission, the vital sign information is read out from the memory and output to the character superimposing unit 151.

On the other hand, in cases where the communications control unit 153 confirms that the communications operating state of an I/F part is “in execution”, the communications control unit 153 performs a determination as to the degree of importance of the communications data in execution and the vital sign information. For example, in cases where the communications data in execution is operating parameter information for a medical device, it is determined that the vital sign information has a higher degree of importance, and the communications control unit 153 outputs an interrupt signal (that is used to instruct the CPU 155 to perform interrupt processing) to the CPU 155.

On the basis of this interrupt signal, the CPU 155 reads out the vital sign information from the memory via the system bus 154, and execute processing that displays the vital sign information or that displays information relating to the vital sign information.

Thus, the CPU 155 is arranged so that in cases where communications states do not overlap, communications processing is delegated to the communications control unit 153 inside the FPGA, and in cases where communications states are overlapped, interrupt processing is performed using interrupt signals in accordance with the degree of importance of the communications data. Specifically, the communications processing of data with a low degree of importance may be delayed and temporarily caused to wait, or in cases where data with a low degree of importance is updated during this waiting period, the processing may be compressed even if this data is communicated. Furthermore, the RAM 158 may also be used instead of the abovementioned memory.

[Merits]

Thus, since the communications control unit is responsible for a portion of the communications processing, the processing load on the CPU can be reduced; furthermore, since the CPU performs interrupt processing in accordance with the degree of importance of the communicated information, the communications processing of information with a high degree of importance can be performed quickly.

[Second Embodiment]

Next, a second embodiment of the present invention will be described.

The second embodiment illustrates a configuration in which the interrupt processing and priority determination processing described in the first embodiment are realized by the multi-task function of a real-time OS (operating system).

Furthermore, parts that are the same as in the construction of the first embodiment are labeled with the same symbols, and a description of such parts is omitted.

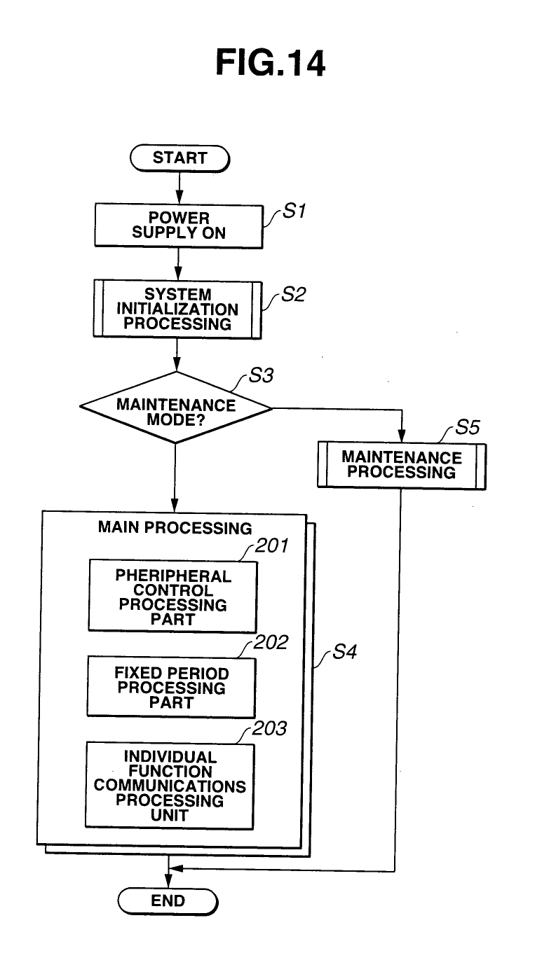

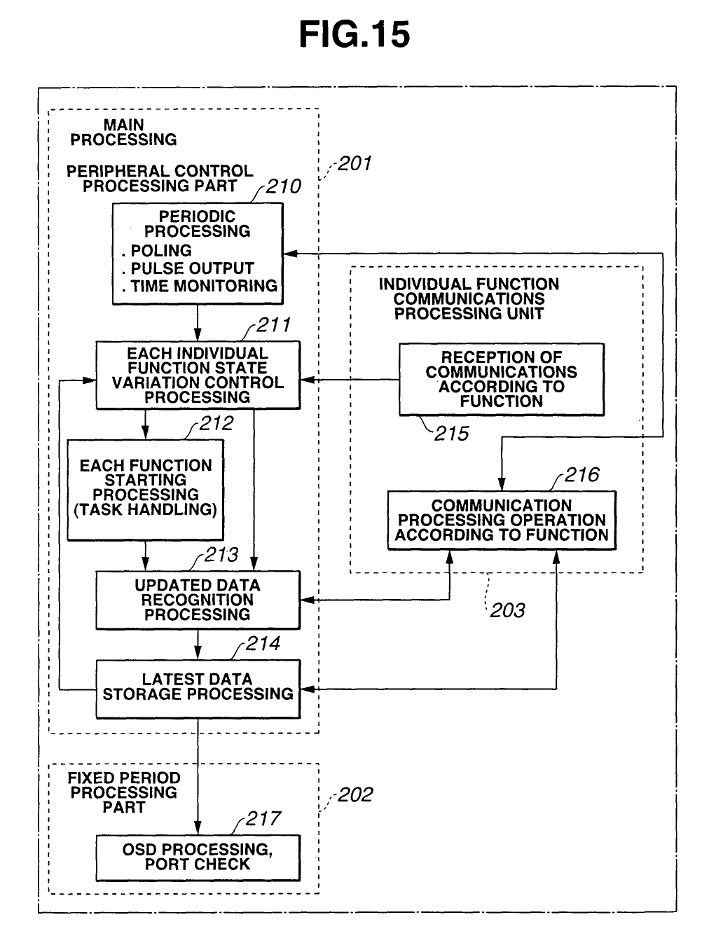

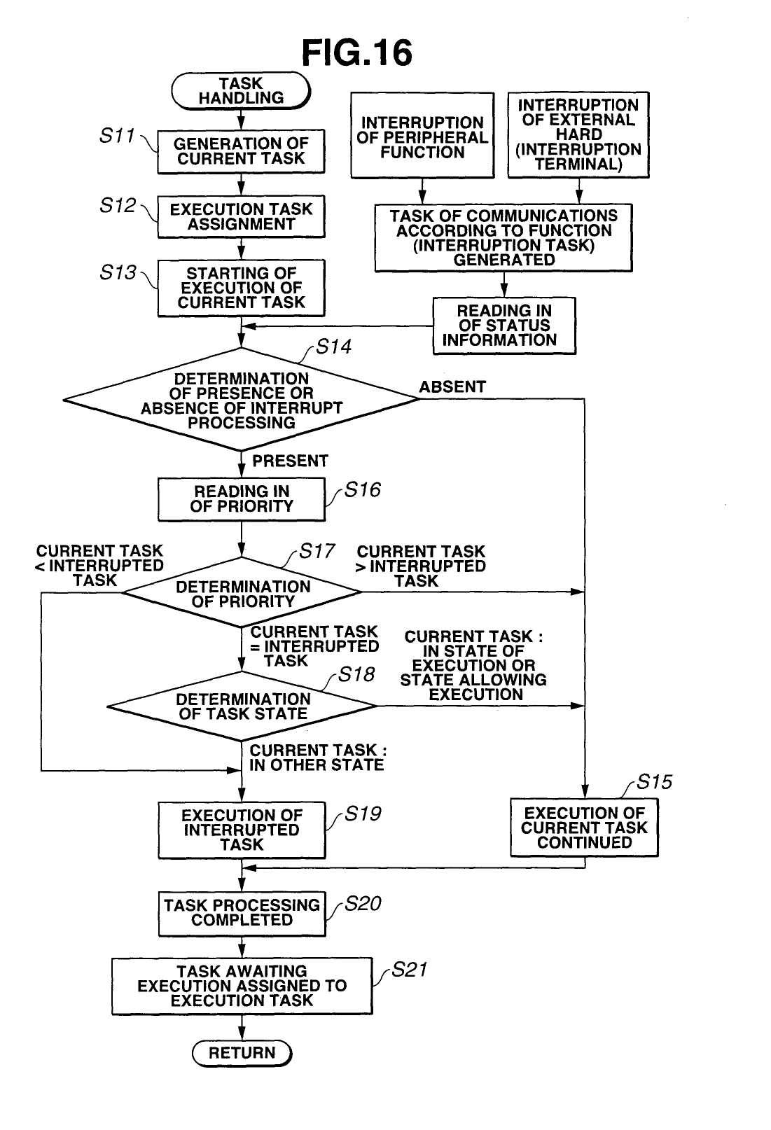

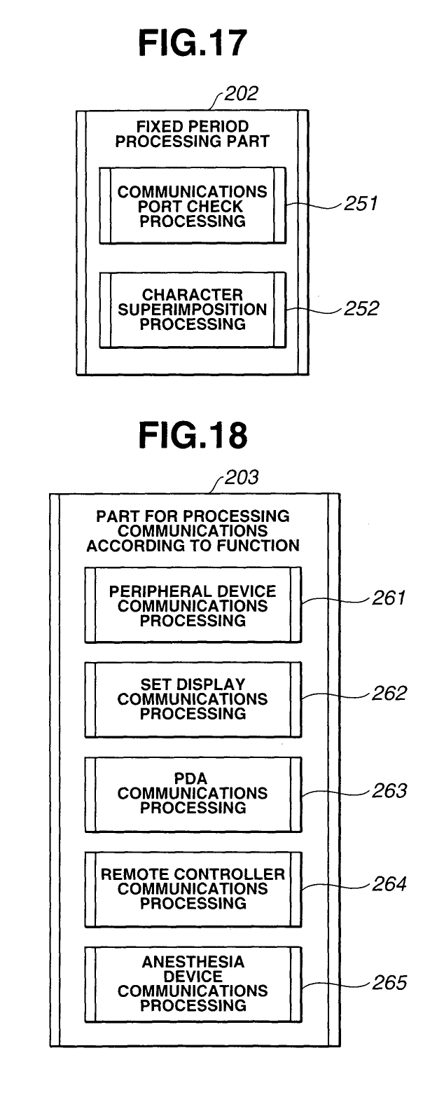

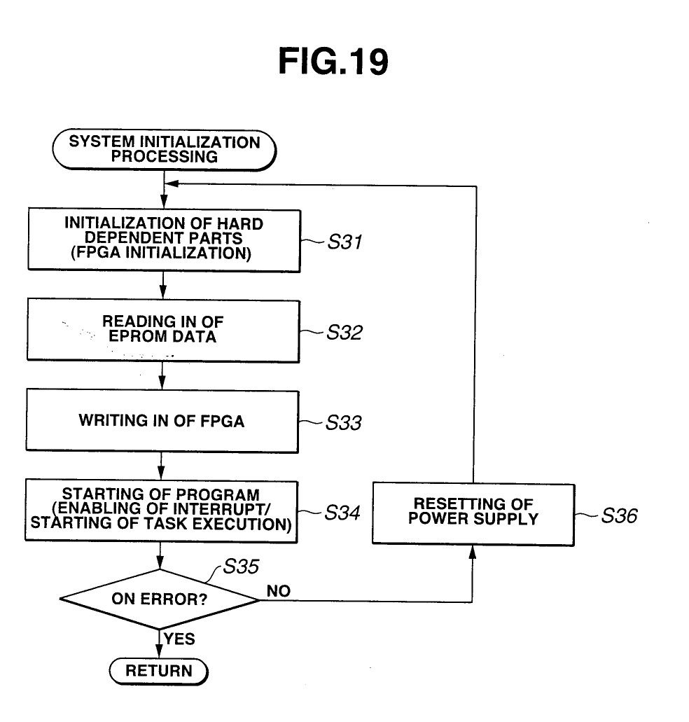

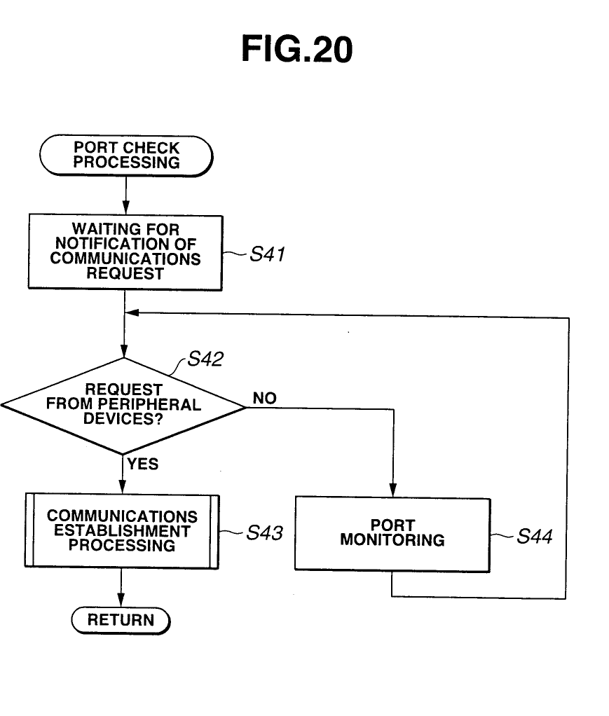

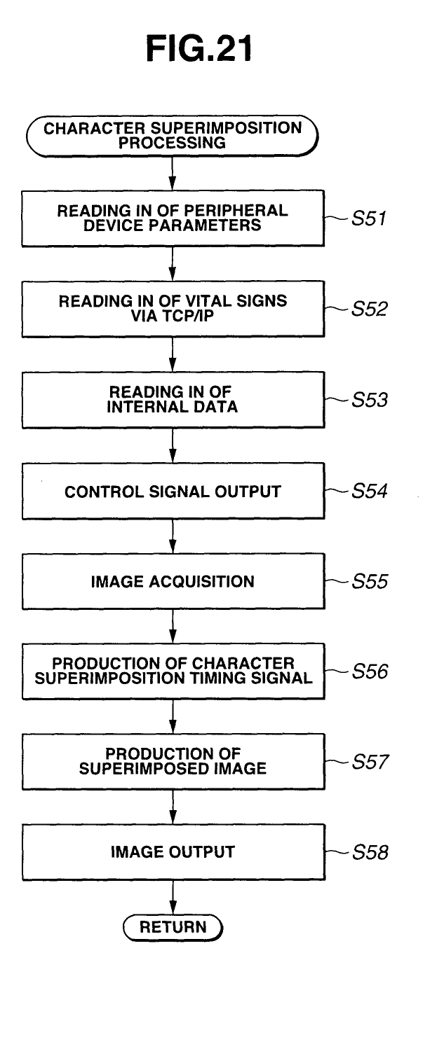

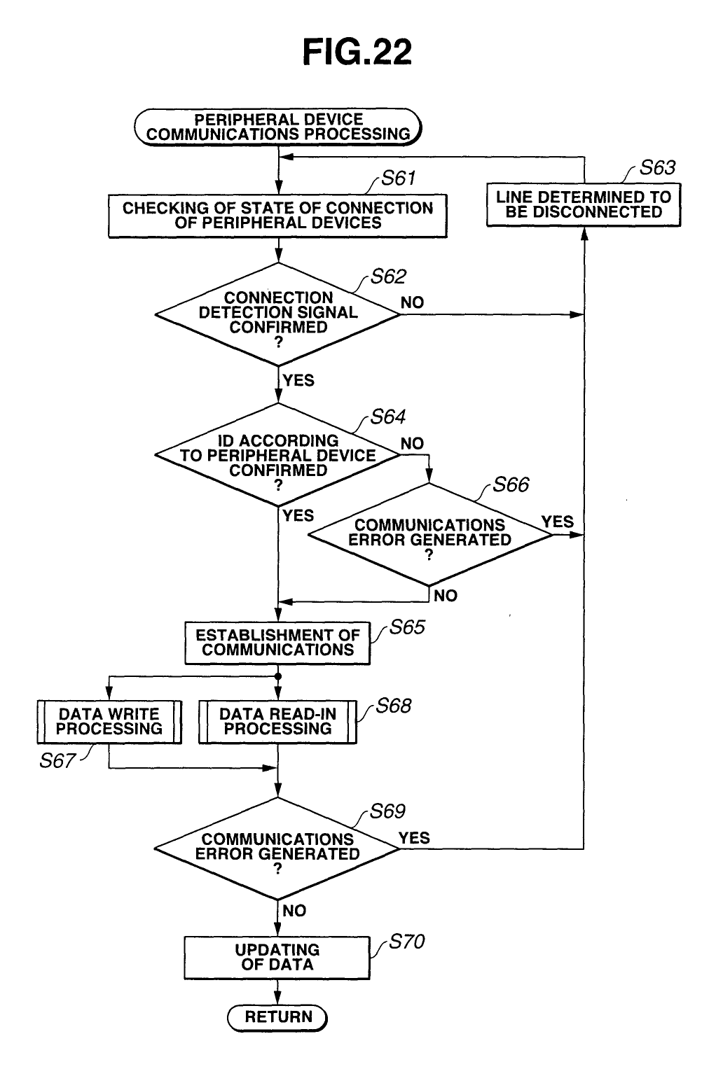

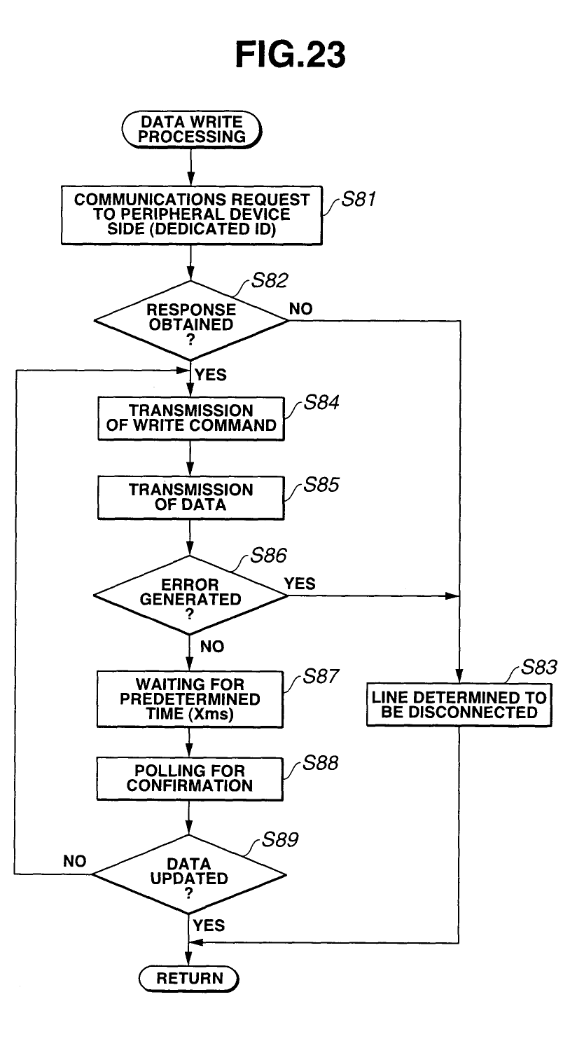

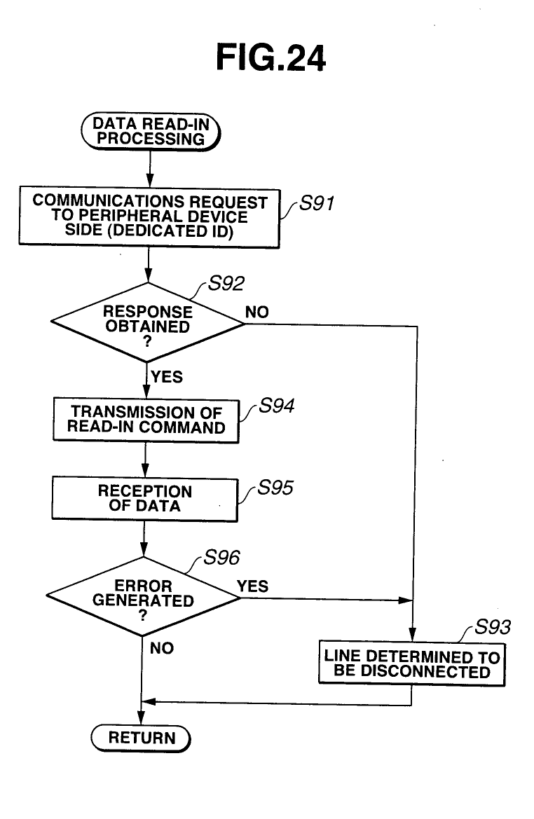

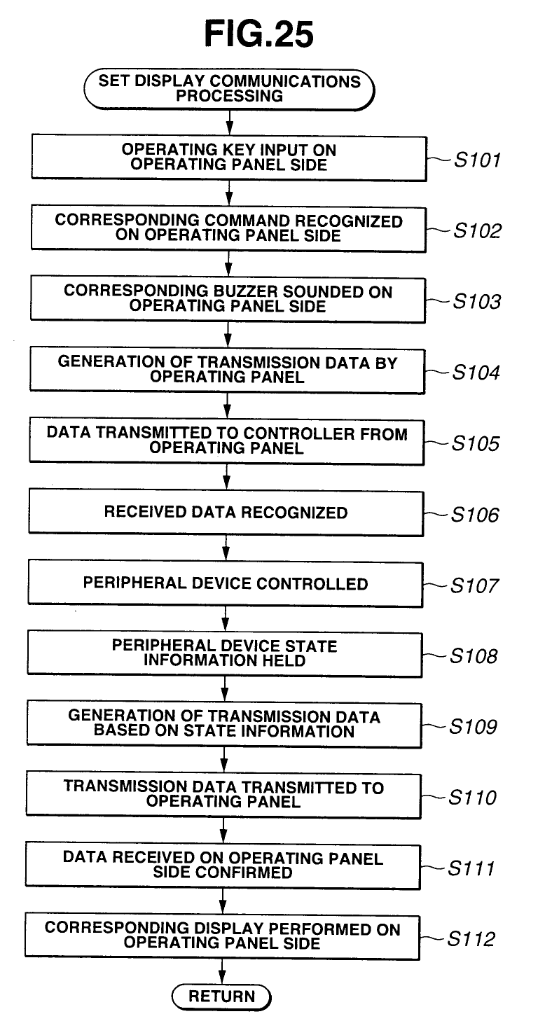

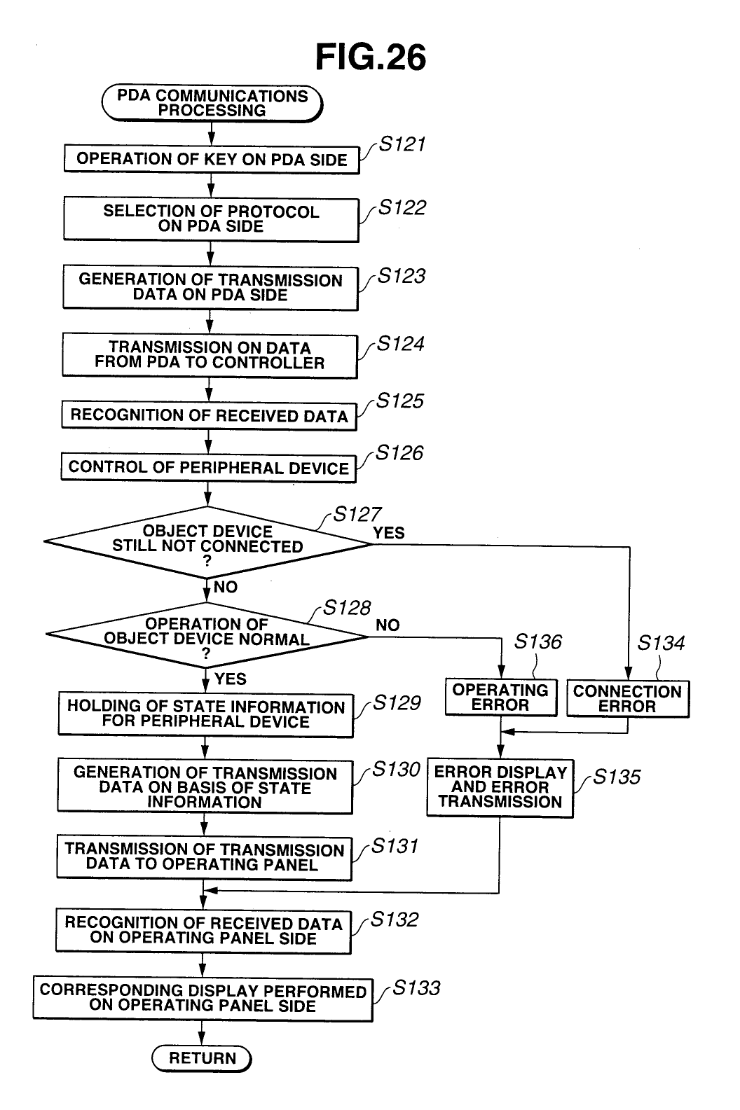

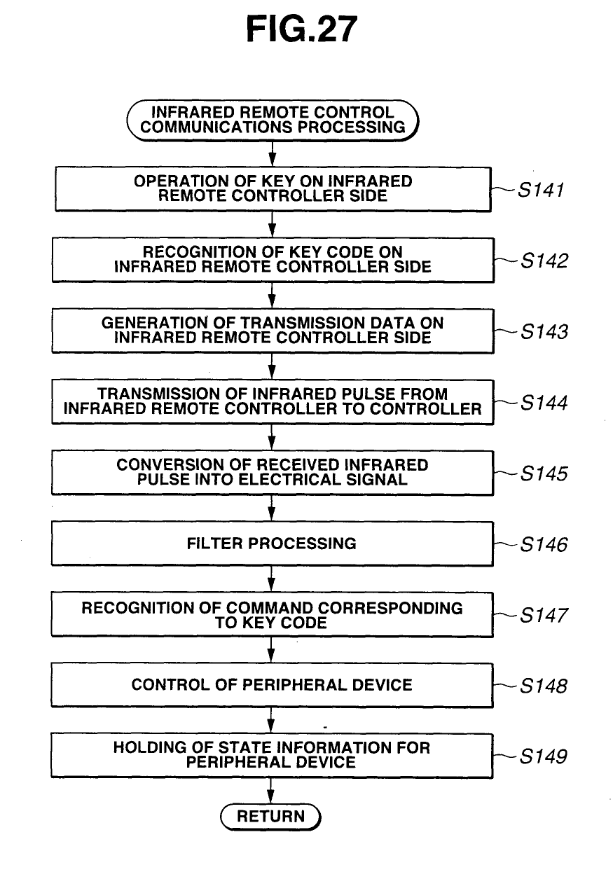

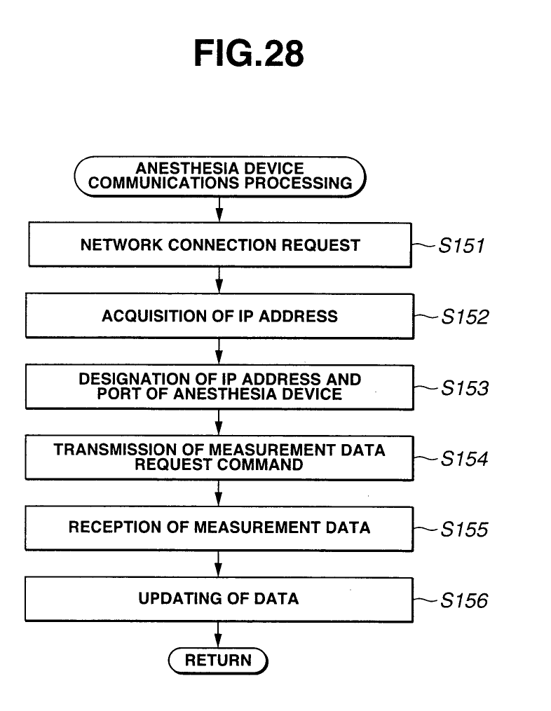

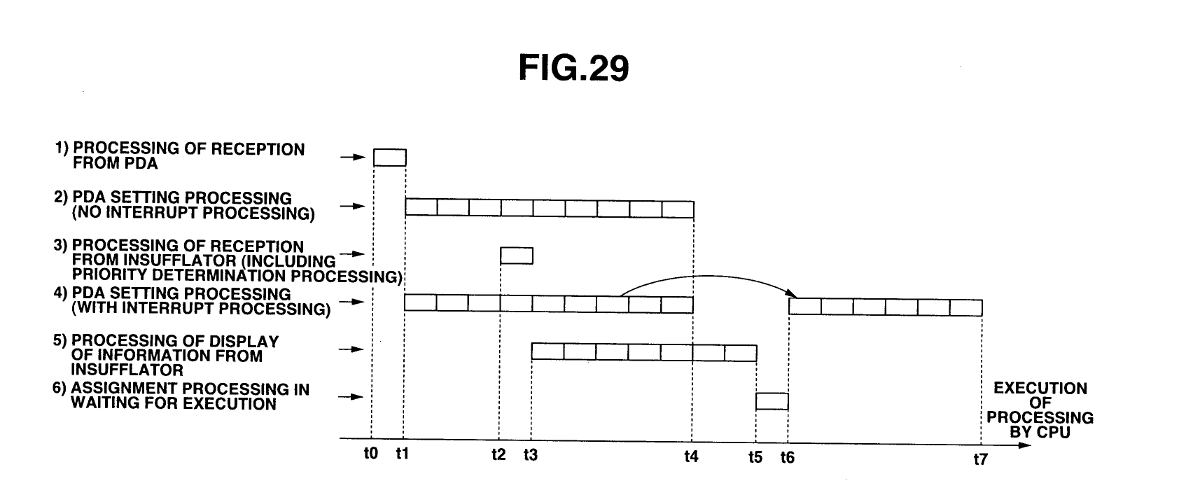

FIGS. 14 through 32 relate to the second embodiment of the present invention. FIG. 14 is a flow chart which shows the overall flow of the processing of the CPU 155, FIG. 15 is a diagram which illustrates the main processing in FIG. 14, FIG. 16 is a flow chart which shows the flow of the task handling processing in FIG. 15, FIG. 17 is a diagram which illustrates the fixed-period processing part in FIG. 14, FIG. 18 is a diagram which illustrates an individual function communications processing unit in FIG. 14, FIG. 19 is a flow chart which shows the flow of system initialization processing in FIG. 14, FIG. 20 is a flow chart which shows the flow of the communications port checking processing of the fixed-period processing part shown in FIG. 17, FIG. 21 is a flow chart which shows the flow of the character superimposition processing of the fixed-period processing part shown in FIG. 17, FIG. 22 is a flow chart which shows the flow of the peripheral device communications processing of the individual function communications processing unit shown in FIG. 18, FIG. 23 is a flow chart which shows the data write processing in FIG. 22, FIG. 24 is a flow chart which shows the data read-in processing in FIG. 22, FIG. 25 is a flow chart which shows the flow of the setting display communications processing of the individual function communications processing unit shown in FIG. 18, FIG. 26 is a flow chart which shows the flow of the PDA communications processing of the individual function communications processing unit shown in FIG. 18, FIG. 27 is a flow chart which shows the flow of the remote controller communications processing of the individual function communications processing unit shown in FIG. 18, FIG. 28 is a flow chart which shows the flow of the anesthetic device communications processing of the individual function communications processing unit shown in FIG. 18, FIG. 29 is a first time chart which is used to illustrate the flow charts of FIGS. 15 and 16, FIG. 30 is a second time chart which is used to illustrate the flow charts of FIGS. 15 and 16, FIG. 31 is a third time chart which is used to illustrate the flow charts of FIGS. 15 and 16, and FIG. 32 is a fourth time chart which is used to illustrate the flow charts of FIGS. 15 and 16.

[Construction]

The communications process and protocol analysis described in the first embodiment are performed by the CPU 155. Here, an operating system (hereafter referred to as “OS”) is accommodated in the EEPROM 156. The OS is loaded into the RAM 158 when the system controller 22 is started, resulting in a state in which individual function starting processing can be executed as a multi-task function (described later) by the CPU 155 executing the OS.

[Operation]

Here, the processing of the CPU 155 of the system controller 22 will be described. As is shown in FIG. 14, when the power supply is switched on in step S1, system initialization processing (described later) is executed in step S2. Then, in step S3, a determination is made as to whether the mode is the maintenance mode or not. In cases where the mode is not the maintenance mode, the main processing is performed in step S4, and processing is ended. In cases where the mode is the maintenance mode, predetermined maintenance processing is executed in step S5, and processing is ended. The main processing comprises the peripheral control processing part 201, fixed-period processing part 202 and individual function communications processing unit 203.

In the system initialization processing of step S2, as is shown in FIG. 19, the CPU 155 of the system controller 22 initializes the hardware-dependent parts, i.e., initializes the character superimposing unit 151, setting operating unit I/F unit 152, unidirectional infrared I/F unit 67a, bidirectional infrared I/F unit 66a and serial communications I/F unit 150 constructed from the FPGA. Then, in step S32, the setting data for the FPGA is read in from the EEPROM 156, and in step S33, the setting data is written into the character superimposing unit 151, setting operating unit I/F unit 152, unidirectional infrared I/F unit 67a, bidirectional infrared I/F unit 66a and serial communications I/F unit 150 constructed from the FPGA. In step S34, the program is stared (interrupt permitted/task execution initiated), and in step S35, a determination is made as to whether or not there is an error. When there is no error, the processing is ended; when there is an error, the power supply is reset in step S36, and the processing returns to step S31.

To describe this in greater detail, in the main processing, as is shown in FIG. 15, periodic processing, state variation control processing for respective functions, starting processing according to respective functions (task handling), updated data recognition processing and latest data storage processing are performed in the peripheral control processing part 201, and the execution of the fixed-period processing part 202 and individual function communications processing unit 203 is controlled.

Here, as is shown in FIG. 16, the task handling of the peripheral control processing part 201 is performed as follows: specifically, when a current task to be executed is generated in step S11, the current task is assigned to a task to be executed in step S12, and execution of the current task is initiated in step S13. On the other hand, when a peripheral function interrupt or external hardware interrupt is input, so that an individual function communications task (interrupt task) is generated, the status of this task is read in so that the processing shifts from step S14 to step S16. The priority of the task is read in in step S16, and the respective priorities of the current task and interrupt task are determined in step S17.

In cases where the priority of the current task is higher than the priority of the interrupt task, the execution of the current task is continued in step S15, and the processing proceeds to step S20. In cases where the priority of the current task and the priority of the interrupt task are the same, the status of the current task is determined in step S18.

In cases where the current task is in a state of execution or an executable state in step S18, the execution of the current task is continued in step S15; in all other cases, the interrupt task is executed in step S19, and the processing proceeds to step S20. Furthermore, in cases where the priority of the current task is lower than the priority of the interrupt task in step S17, the processing proceeds to step S19.

The task processing is ended in step S20; in step S21, the task awaiting execution is assigned to the task that is to be executed, and processing is ended.

In the fixed-period processing part 202, as is shown in FIG. 17, communications port checking processing 251 and character superimposition processing 252 are performed. Details will be described later.

Furthermore, in the individual function communications processing unit 203, as is shown in FIG. 18, peripheral device communications processing 261, setting display communications processing 262, PDA communications processing 263, remote controller communications processing 264 and anesthetic device communications processing 265 are performed. Details will be described later.

In the communications port checking processing 251 of the fixed-period processing part 202, the CPU 155 of the system controller 22 waits for notification of communications requests from the character superimposing unit 151, setting operating unit I/F unit 152, unidirectional infrared I/F unit 67a, bidirectional infrared I/F unit 66a and serial communications I/F unit 150 by monitoring the ports in step S41 as shown in FIG. 20. In step S42, a determination is made as to whether or not there has been a communications request from a peripheral device. In cases where there has been a communications request, communications establishment processing is performed in step S43, and processing is ended. In cases where there has been no communications request, the monitoring of the ports is continued in step S44, and the processing returns to step S42.

In the character superimposing processing 252 of the fixed-period processing part 202, the CPU 155 of the system controller 22 reads in peripheral device parameters from the character superimposing unit 151, setting operating unit I/F unit 152, unidirectional infrared I/F unit 67a, bidirectional infrared I/F unit 66a and serial communications I/F unit 150 in step S51 as shown in FIG. 21. Furthermore, in step S52, vital signs of the patient are read in from the patient monitoring system 4 via the CP/IP. Then, in step S53, internal data is read in, and in step S54, a control signal is output to the peripheral device. When an image is acquired in step S55, a character superimposition timing signal is generated in step S56, a superimposed image in which characters are superimposed on the image is generated in step S56, this image is output in step S57, and processing is ended.

In the peripheral device communications processing 261 of the individual function communications processing unit 203, the CPU 155 of the system controller 22 checks the connection state of the peripheral devices in step S61, and makes a determination as to whether or not a connection detection signal has been confirmed in step S62, as is shown in FIG. 22. In cases where a connection detection signal cannot be confirmed, it is determined in step S63 that the circuit is disconnected, and the processing returns to step S61.

When a connection detection signal can be confirmed, a determination is made in step S64 as to whether or not the device ID has been confirmed. When the device ID can be confirmed, a determination is made in step S65 that communications have been established.

In cases where the device ID cannot be confirmed, a determination is made in step S66 as to whether or not a communications error has occurred. In cases where a communications error has occurred, the processing proceeds to step S63; in cases where a communications error has not occurred, the processing proceeds to step S65.

When it is determined that communications have been established, the data write processing (described later) of step S67 or the data read-in processing (described later) of step S68 is executed, and a determination is made in step S69 as to whether or not a communications error has occurred. In cases where a communications error has occurred, the processing proceeds to step S63, while in cases where a communications error has not occurred, the data is updated in step S70, and processing is ended.

In the data write processing of step S67, the CPU 155 of the system controller 22 sends a communications request to the peripheral device side in step S81, and makes a determination as to whether or not there has been a response from the peripheral device in step S82, as is shown in FIG. 23. In cases where there is no response from the peripheral device, a determination is made in step S83 that the circuit is disconnected, and processing is ended.

When there is a response from the peripheral device, a write command is transmitted to the peripheral device in step S84, and data is transmitted to the peripheral device in step S85. Then, a determination is made in step S86 as to whether or not an error has occurred. In cases where an error has occurred, the processing proceeds to step S83, while in cases where no error has occurred, the processing waits for a predetermined time in step S87, and then performs polling for the purpose of confirmation in step S88. In step S89, a determination is made as to whether or not the data of the peripheral device has been updated; in cases where the data has been updated, the processing is ended, while in cases where the data has not been updated, the processing returns to step S84.

In the data read-in processing of step S68, the CPU 155 of the system controller 22 sends a communications request to the peripheral device side in step S91, and makes a determination as to whether or not there has been a response from the peripheral device in step S92, as is shown in FIG. 24. In cases where there is no response from the peripheral device, a determination is made in step S93 that the circuit is disconnected, and processing is ended.

When there is a response from the peripheral device, a read-in command is transmitted to the peripheral device in step S94, and data is received from the peripheral device in step S95. Then, in step S96, a determination is made as to whether or not an error has occurred. In cases where an error has occurred, the processing proceeds to step S93, and in cases where an error has not occurred, the processing is ended.

In the setting display communications processing 262 of the individual function communications processing unit 203, as is shown in FIG. 25, when an operating key is input on the side of the operating panel 21 in step S101, a corresponding command is recognized on the side of the operating panel 21 in step S102, and a corresponding buzzer is caused to sound on the side of the operating panel 21 in step S103. Then, transmission data is generated by the operating panel 21 in step S104, and data is transmitted from the operating panel 21 to the CPU 155 of the system controller 22 in step S105.

The CPU 155 of the system controller 22 recognizes the received data in step S106, controls the peripheral device in step S107, holds the state information for the peripheral device in step S108, and creates transmission data on the basis of the state information in step S109.

Then, in step S110, the transmission data is transmitted to the operating panel 21, and in step S111, the received data is recognized on the side of the operating panel 21. In step S112, a display corresponding to the received data is performed on the side of the operating panel 21, and the processing is ended.

In the PDA communications processing 263 of the individual function communications processing unit 203, as is shown in FIG. 26, when a key is operated on the side of the PDA 68 in step S121, a protocol (IrDa or serial communications) is selected on the side of the PDA 68 in step S122, transmission data is generated on the side of the PDA 68 in step S123, and data is transmitted from the PDA 68 to the CPU 155 of the system controller 22 in step S124.

The CPU 155 of the system controller recognizes the received data in step S125, controls the peripheral device in step S126, and determines the connection state of the peripheral device in step S127. In cases where the peripheral device is connected, a determination is made in step S128 as to whether or not the operation of the peripheral device is normal. In cases where this operation is normal, the setting information of the peripheral device is held in step S129, and transmission information is created on the basis of the setting information in step S130.

Then, the transmission data is transmitted to the peripheral device, and in step S131, the transmission data is transmitted to the operating panel 21. In step S132, the received data is recognized on the side of the operating panel 21; in step S133, a display corresponding to the received data is performed on the side of the operating panel 21, and the processing is ended.

When it is determined in step S127 that the peripheral device is unconnected, it is determined in step S134 that a connection error has occurred; then, in step S135, an error is displayed, and error information is transmitted to the PDA 68. The processing then proceeds to step S132.

Furthermore, when it is determined in step S128 that the operation of the peripheral device is abnormal, it is determined in step S136 that an operating error has occurred, and the processing proceeds to step S135.

Furthermore, a construction may also be used in which the processing from step S127 on is executed by the abovementioned peripheral device communications processing 261 and setting display communications processing 262.

In the remote controller communications processing 264 of the individual function communications processing unit 203, as is shown in FIG. 27, when a key is operated on the side of the infrared remote controller 69 in step S141, the key code is recognized on the side of the infrared remote controller 69 in step S143, transmission data is created on the side of the infrared remote controller 69 in step S143, and data is transmitted as infrared pulses from the infrared remote controller 69 to the system controller 22 in step S144.

In the system controller 22, the received infrared pulses are converted into an electrical signal in step S145, and predetermined filter processing is performed in step S146, so that a command corresponding to the key code is recognized in step S147, and the peripheral device is controlled in step S148. The state information of the peripheral device is held in step S149, and the processing is ended.

In the anesthetic device communications processing 265 of the individual function communications processing unit 203, as is shown in FIG. 28, the CPU 155 of the system controller 22 requests a network connection to the hospital LAN 101 for the hospital server 113 in step S151, and acquires an IP address in step S152. Then, the IP address and port of the anesthetic device (not shown in the figures) connected to the hospital LAN 101 are designated in step S153, and a request command for measurement data is sent out to the anesthetic device in step S154.

Then, when the measurement data is received from the anesthetic device in step S155, the data is updated in step S156, and the processing is ended.

The above has been a description of the flow of the operations of the respective functions; next, the operation of the peripheral control processing part 201 and individual function communications processing unit 203 will be described using a more concrete example.

For example, in cases where PDA communications processing 263 is executed, the processing of the peripheral processing part 201 is performed in step S125.

To describe this in greater detail, when information is received from the PDA 68, the received and processed information is transmitted from an individual function communications reception 215 to an each individual function state variation control processing 211 in FIG. 15. The each individual function state variation control processing 211 recognizes that a variation in state has occurred, and the processing proceeds to an each function starting processing 212. In the each function starting processing 212, the processing of the respective steps of the PDA communications processing 263 is assigned as tasks, and the tasks are executed. In the recognition processing 213, the data produced by the executed tasks is recognized as updated data, and in the latest data storage processing 214, storage processing of the updated data is performed; then, the each individual function state variation control processing 211 is informed that there has been a change in the stored data.

Furthermore, the operation of the each function starting processing 212 in a case where the PDA communications processing 263 is executed will be described with reference to FIG. 29.

For example, when step S121 through step S124 of the PDA communications processing 263 is executed on the side of the PDA 68, and step S124 is executed at a timing of t0, reception processing is performed on the side of the controller 22 from t0 to t1. Specifically, reception is performed by the bidirectional I/F part 66a, and when reception is completed by the bidirectional I/F part 66a, step S125 of the PDA communications processing 263 is executed in the CPU 155, and the processing of the abovementioned peripheral control part 201 is executed.

Then, from t1, the processing of step S126 of the PDA communications processing 263 is initiated, and in cases where there is no interrupt processing, the processing of step S126 through step S133 is executed from t1 to t4.

Furthermore, when (for example) when the reception of abdominal cavity overpressure warning information is initiated by the serial communications I/F 150 from the insufflator 14 at a timing of t2 in cases where it appears that interrupt processing will be performed, the processing from step S216 on in the PDA communications processing 263 that is in execution is temporarily stopped, and reception processing from the insufflator 14 is executed on the side of the controller 22 from t2 to t3. Specifically, peripheral device communications processing 261 is executed in the CPU 155. Furthermore, in the CPU 155, the processing of the peripheral control processing part 201 is executed, and from t2 to t3, the CPU 155 determines that there is interrupt processing in step S14 of the each function starting processing 212, and proceeds to step S16. In step S16, for example, priority information according to the type of communications protocol (RS232C>IrDA) is stored beforehand in an EEPROM or the like, and this priority information is read in. When a determination is made to proceed to step S19 in the determination results of step S17, the CPU 155 executes interrupt processing in step S19 of the each function starting processing 212, and the controller 22 initiates display processing in which abdominal cavity overpressure warning information transmitted from the insufflator 14 is displayed on the display device 19 at a timing of t3. The abovementioned display processing is ended by the execution of step S20 of the each function starting processing 212 at a timing of t5. Step S21 of the each function starting processing 212 is executed between t5 and t6, and the previously stopped PDA communications processing 263 is re-initiated at a timing of t6, so that the PDA communications processing 263 is executed between t6 and t7.

Furthermore, in cases where there are three or more communications protocols as in the embodiments of the present application, processing may be performed (for example) as follows: specifically, priority information which is such that (for example) TCP/IP>RS232C>IrDA is stored in memory, and when vital sign information based on TCP/IP is received during the abovementioned interrupt processing, further interrupt processing is performed, so that the interrupt processing is multiplexed.

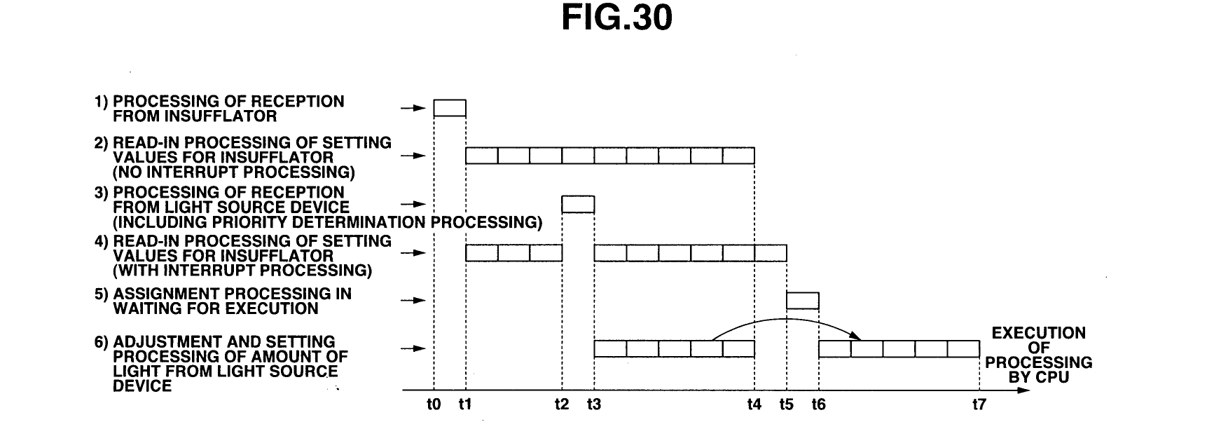

Furthermore, in cases where there are a plurality of communications protocols of the same type as in the embodiments of the present application, priority information corresponding to the type of device is stored in memory, and, as is shown in FIG. 30 (for example), when an adjustment of the light quantity of the light source device 16 occurs during the measurement value read-in processing of the insufflator 14, a determination of the priority of the each function starting processing 212 is made by reception processing from the light source device performed between t2 and t3 on the basis of the previously stored priority information corresponding to the peripheral devices (insufflator 14>light source device 16). Then, the processing of the insufflator 14 is continued from t3 (step S15 of the each function starting processing 212), and after the continued processing is completed at t5, the task assignment processing of the light source device 16 is performed between t5 and t6, and the processing of the light source device 16 is executed from t6 on.

Furthermore, in cases where further information is received from the light source device 16 between t3 and t5, the received data that was in a state awaiting execution is overwritten, and the most recent data may be processed.

Furthermore, a case in which priority information corresponding to the functions of the medical devices is stored in memory, and communications processing is performed for each function of the medical devices, will be described.

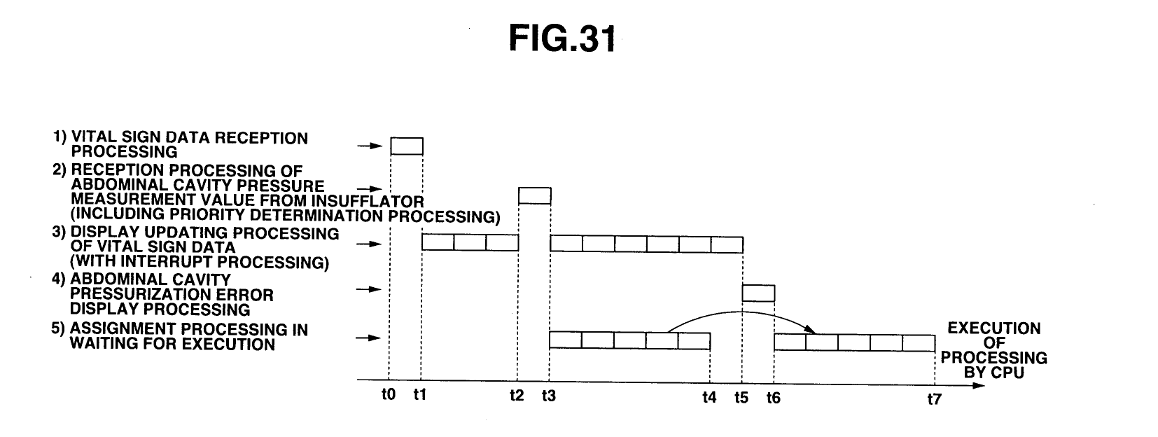

For example, priority data which is such that abdominal cavity overpressure>vital sign information display processing>abdominal cavity pressure measurement value updating processing is stored in memory beforehand, and, as is shown in FIG. 31, in a case where processing in which the measurement value of the abdominal cavity pressure is received from the insufflator 14 and displayed at a timing of t2, at which processing that receives vital sign information in the TCP/IP protocol and displays this received information on the monitor is being performed, when the vital sign information is received at a timing of t0, vital sign information display updating processing to the monitor is initiated from t1, the vital sign information display updating processing is temporarily stopped at t2, the priorities of the vital sign information display updating processing and the measurement value updating processing of the insufflator 14 are determined between t2 and t3, the vital sign information display updating processing is continued between t3 and t5, and the abdominal cavity display processing is executed between t6 and t7.

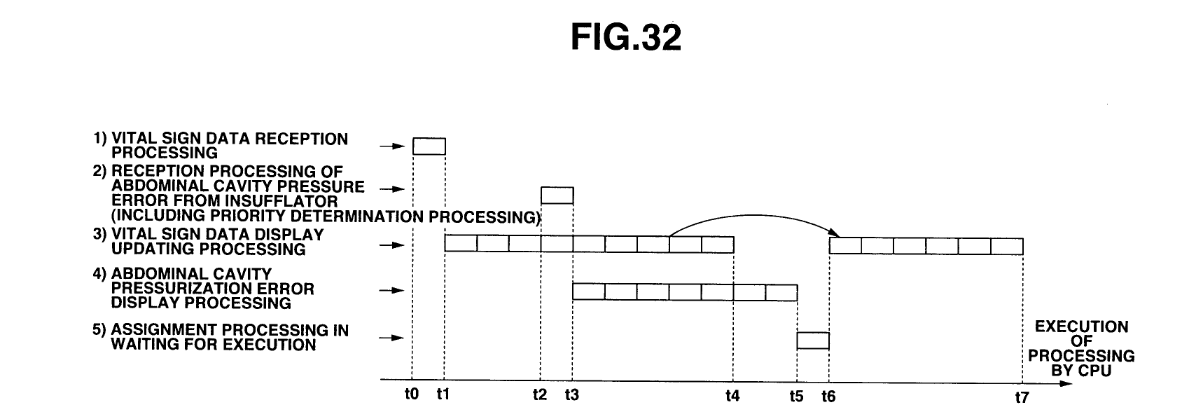

Furthermore, as is shown in FIG. 32, vital sign information is received in the same manner as in FIG. 31, and in a case where processing which receives an abdominal cavity overpressure error from the insufflator 14 and displays this on the monitor occurs at a timing of t2, at which processing that displays the vital sign information on the monitor is being performed, the vital sign information is received at a timing of t0, vital sign information display updating processing to the monitor is initiated from t1, and the vital sign information display updating processing is temporarily stopped at t2. The priorities of the vital sign information display updating processing and the abdominal cavity overpressure error warning processing of the insufflator 14 are determined between t2 and t3, the abdominal cavity overpressure error warning processing is executed between t3 and t5, and the temporarily stopped vital sign information display updating processing is executed between t6 and t7.

Thus, priority information corresponding to each function of the medical devices can be stored in memory, and communications control processing based on the priority information corresponding to each function of the medical devices can be performed.

[Merits]

As was described above, the present invention possesses the following merits: specifically, using the multi-task function of the OS when communications processing and protocol analysis are performed with a plurality of medical devices, interrupt processing can be performed in accordance with the priority of the task, the processing order can be optimally rearranged, and processing that has become unnecessary in this case can be discarded, so that processing can be efficiently performed.

Furthermore, as was described above, the first and second embodiments possess the following merits: specifically, even in the case of communications with a plurality of devices that have different communications formats, the control of the plurality of devices with different communications formats can be quickly accomplished without increasing the cost or increasing the size of the apparatus.

[Third Embodiment]

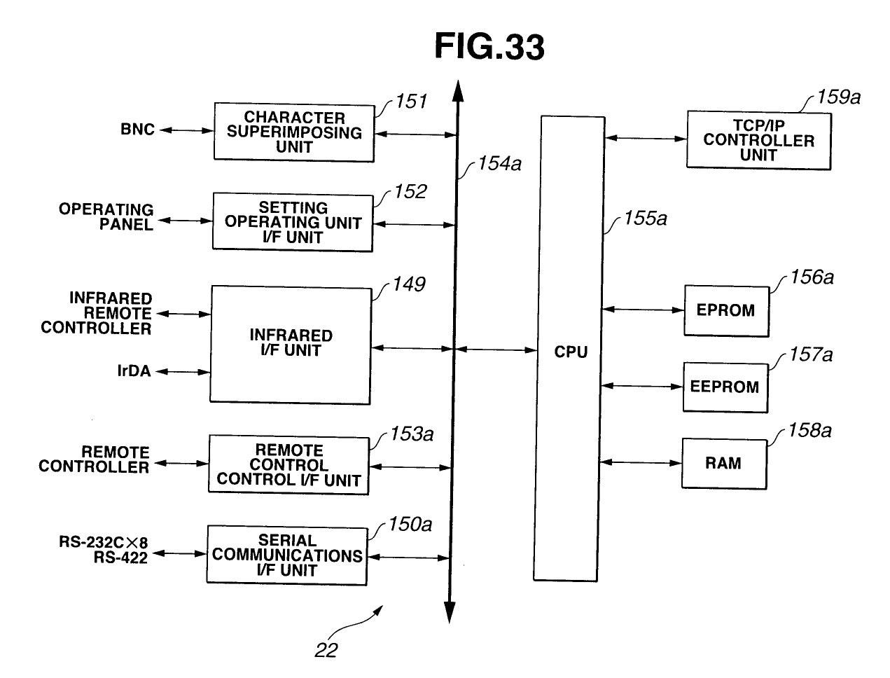

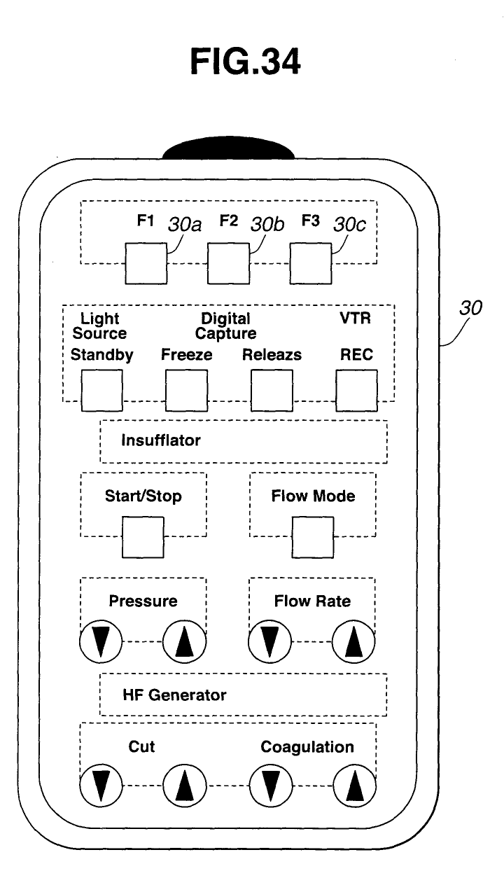

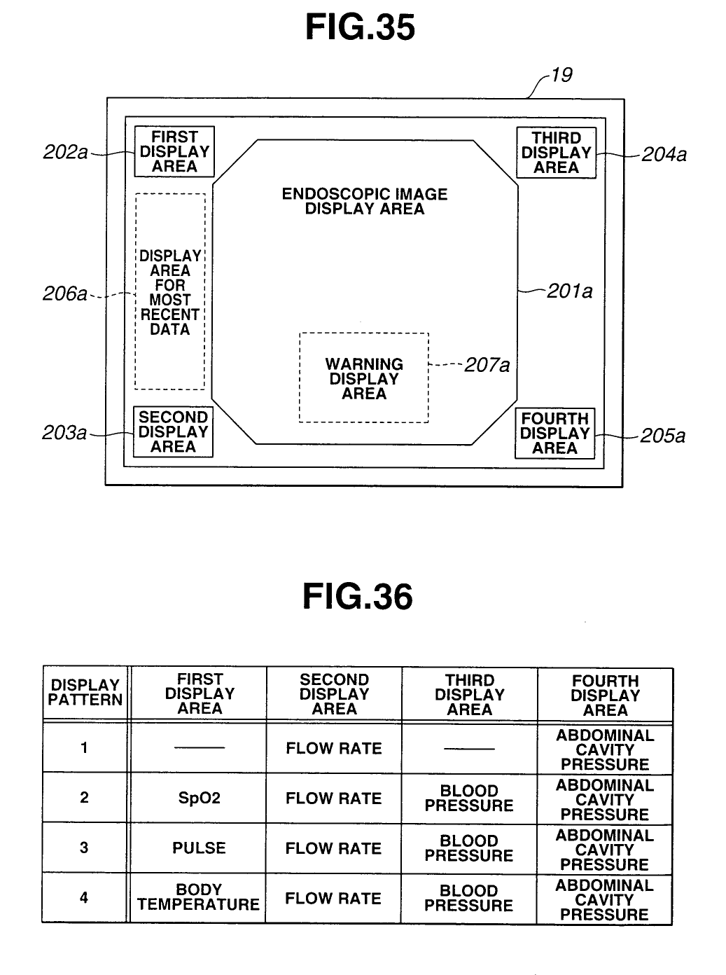

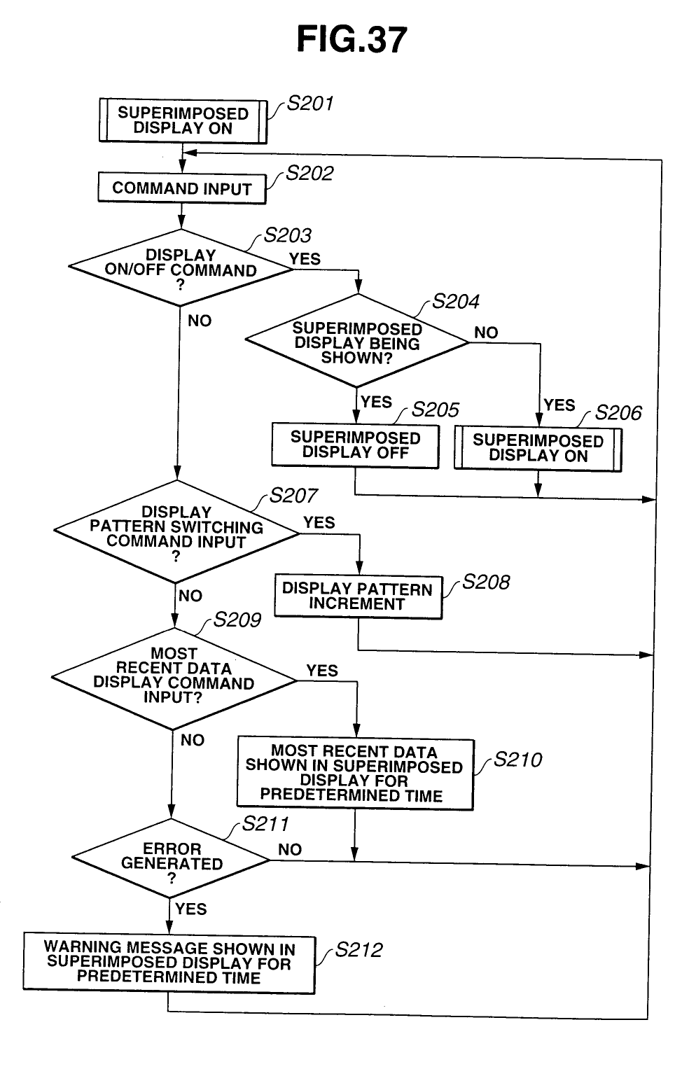

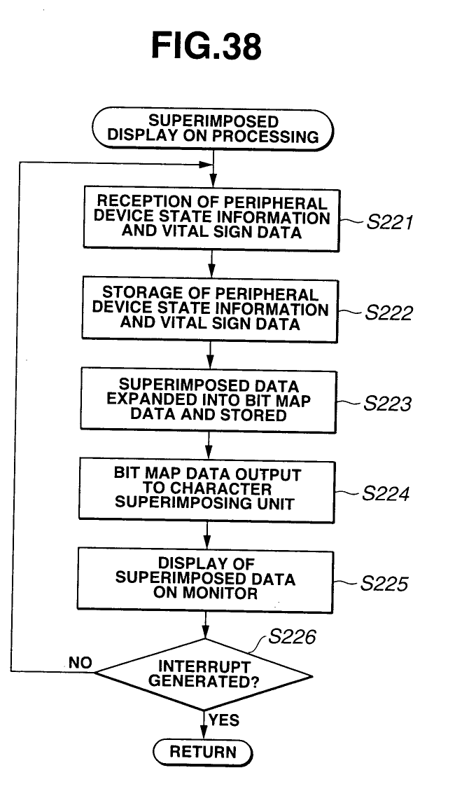

FIGS. 33 through 37 relate to a third embodiment of the present invention. Furthermore, since the overall construction of the system is similar to the constructions of the first and second embodiments, the same constituent elements will be labeled with the same symbols in the description of the present embodiment, and a description of these constituent elements will be omitted. Accordingly, only items in the present embodiment that differ from the first and second embodiments will be described. FIG. 33 is a block diagram which shows the construction of the system controller 22 shown in FIG. 1, FIG. 34 is a diagram which shows the construction of the remote controller shown in FIG. 1, FIG. 35 is a diagram which shows the display screen of the display device that displays endoscopic images (shown in FIG. 1), FIG. 36 is a diagram which illustrates the pattern of the superimposed data that is superimposed on the display screen shown in FIG. 35, FIG. 37 is a flow chart which illustrates the operation of the system controller shown in FIG. 1, and FIG. 38 is a flow chart which shows flow of the superimposed display ON processing in FIG. 37.

[Construction]

As was described above, the overall construction of the present embodiment is similar to the constructions of the first and second embodiments; accordingly, only items that differ from the first and second embodiments will be described below.

The remote controller 30 is constructed as shown in FIG. 34, and is a second concentrated operating device that is operated by an operating surgeon in a sterile area. This device is arranged so that other devices with which communications have been established can be operated via the system controller 22.

In the third embodiment, as is shown in FIG. 5, a power supply switch 131, the abovementioned bidirectional infrared I/F 66 for the PDA 68, and the abovementioned unidirectional infrared I/F 67 for the infrared remote controller 69, are installed on the front surface of the system controller 22, and as is shown in FIG. 6, eight RS-232C communications connectors 135(1) through 135(8) (for example) which are used to control the electrical scalpel 13, insufflator 14, endoscopic camera device 15, light source device 16, VTR 17, concentrated display panel 20 and the like, an RS-422 communications connector 136 which is used to control the remote controller 30, a connector 137 such as a 10BaSe/T or the like which is used for connection with the hospital LAN 101, a BNC 138 which connects the display device 19, a pin jack 139 which transmits and receives video signals to and from the VTR 17, a communications connector 140 which is used for setting control of the operating panel 21 and the like are installed on the back surface of the system controller 22.

As is shown in FIG. 33, the system controller 22 comprises a character superimposing unit 151 which superimposes desired characters on the endoscopic image and outputs this image to the BNC 138, a setting operating unit I/F unit 152 which transmits and receives data to and from the operating panel 21, an infrared I/F unit 149 which performs infrared communications with the infrared remote controller 69 and PDA 68, a remote controller control I/F unit 153a which transmits and receives data to and from the remote controller 30, and a serial communications I/F unit 150a which performs serial communications via the RS-232C communications connectors 135(1) through 135(8) and RS-422 communications connector 136, and is constructed so that these parts are connected to an internal bus 154a.

A CPU 155a which controls the interior of the system controller 22 is connected to the abovementioned internal bus 154a, and the CPU 155a controls the interior of the system controller 22 using an EEPROM 156a, EEPROM 157a, RAM 158a and the like. Furthermore, a TCP/IP control unit 159a is connected to the CPU 155a, and a connection to the hospital LAN is made by the TCP/IP control unit 159a.

Furthermore, in the first and second embodiments, wireless communications are performed using infrared (unidirectional infrared communications and bidirectional infrared communications, e.g., an IrDA system or the like). However, electromagnetic wireless may also be used for the transmission and reception of peripheral device parameters in both directions; e.g., a wireless LAN, Bluetooth or the like may also be used. In this case, since wireless is used, communications can always be continued so that data can be exchanged without being blocked by obstacles.

[Effect]

As is shown in FIG. 34, the three function keys F1, F2 and F3 of the remote controller 30 are a display ON/OFF key 30a which inputs display ON/OFF commands, a display pattern switching key 30b which inputs display pattern switching commands, and a most-recent data display key 30c which inputs most-recent data display commands. By operating these keys 30a, 30b and 30c, it is possible to display pre-registered peripheral device state information and vital sign data in a dispersed display in a first display area 202a disposed at the upper left of the endoscopic image display area 201a of a display device 19 that displays endoscopic images, a second display area 203a disposed at the lower left of the image display area 201a, a third display area 204a disposed at the upper right of the image display area 201a, or a fourth display area 205a disposed at the lower right of the image display area 201a, and to display the most recent state information for peripheral devices for a predetermined period of time in a most-recent data display area 206a, as shown in FIG. 35.

Furthermore, in the display device 19, a warning display area 207a which displays a warning message when the abdominal cavity pressure or output of treatment devices (electrical scalpel or ultrasonic treatment device), or the vital sign data for the patient, departs from preset setting values is superimposed and displayed on the endoscopic image display area 201a.

The peripheral device state information and vital sign data displayed in the first display area 202a, second display area 203a, third display area 204a and fourth display area 205a, are respectively set as a plurality of display patterns, e.g., four display patterns 1, 2, 3 and 4, as shown in FIG. 36, and are stored beforehand in the EEPROM 157a of the system controller 22.

Furthermore, in the system controller 22, as is shown in FIG. 37, the peripheral device state information and vital sign data of display pattern 1 are dispersed and displayed as a superimposed display in the first display area 202a, second display area 203a, third display area 204a and fourth display area 205a in the default case in step S201.

Next, in step S202, the input of the three function keys F1, F2 and F3 of the remote controller 30 is checked, and in step S203, a determination is made as to whether or not the function key F1, i.e., a display ON/OFF command, has been input. When a display ON/OFF command is input, a determination is made in step S204 as to whether or not the display device 19 is showing a superimposed display. In cases where a superimposed display is being shown, the superimposed display is switched OFF in step S205. In case where no superimposed display is being shown, the superimposed display is switched ON in step S206.

Furthermore, in step S207, a determination is made as to whether or not the function key F2, i.e., a display pattern switching command, has been input. When a display pattern switching command is input, the number of the display pattern is incrementally increased in step S208.

Furthermore, in step S209, a determination is made as to whether or not the function key F3, i.e., a most-recent data display command, has been input. When a most-recent data display command is input, the state information of the peripheral device connected to the system controller 22 and the vital sign information are displayed in a superimposed display for a predetermined period of time in the most-recent data display area 206a in step S210.

Furthermore, in step S211, a determination is made as to whether or not an error interrupt has been generated from the peripheral device. When an error interrupt is generated, a warning message is displayed in a superimposed display for a predetermined period of time in the warning display area 207 in step S212.

In the superimposed display ON processing in the abovementioned step S201 and step S206, as is shown in FIG. 38, the peripheral device state information and vital sign data are received in step S221, and the received state information and vital sign data are stored in the RAM 158a in step S222.

Furthermore, in step S223, the superimposed data that is superimposed on the basis of the state information and vital sign data is expanded into bit map data and stored in the RAM 158a, and in step 224, the bit map data is output to the character superimposing unit 151, so that the character superimposing unit 151 displays the superimposed data on the display device (monitor) 19 in step S225.

Furthermore, when the function keys F1, F2 or F3 are pressed in step S226 so that an interrupt is generated, the processing is ended, commands are discriminated, and predetermined processing is performed.

[Merits]

Thus, in the present embodiment, peripheral device state information and vital sign data are dispersed and displayed in a superimposed display in a first display area 202a disposed at the upper left of the endoscopic image display area 201a of the display device 19 that displays endoscopic images, a second display area 203a disposed at the lower left of the image display area 201a, a third display area 204a disposed at the upper right of the image display area 201a, and a fourth image display area 205a disposed at the lower right of the image display area 201a. Accordingly, even if the endoscopic images are displayed at an optimal size, there is no overlapping of the superimposed images with the endoscopic images. Furthermore, the ON/OFF switching of the display of the superimposed images can be accomplished merely by inputting a display ON/OFF command, so that the surgeon can confirm desired peripheral device state information and vital sign data only when this is necessary.

Furthermore, desired peripheral device state information and vital sign data can also be displayed in a superimposed display in desired positions merely by inputting a display pattern switching command.

Furthermore, state information of the peripheral device connected to the system controller 22 and vital sign data can be displayed in a superimposed display for a predetermined period of time by inputting a most-recent data display command; accordingly, the most recent data of the state information of all of the peripheral devices connected to the system controller and all of the vital sign data for the patient can easily be checked on the display device 19.

[Fourth Embodiment]

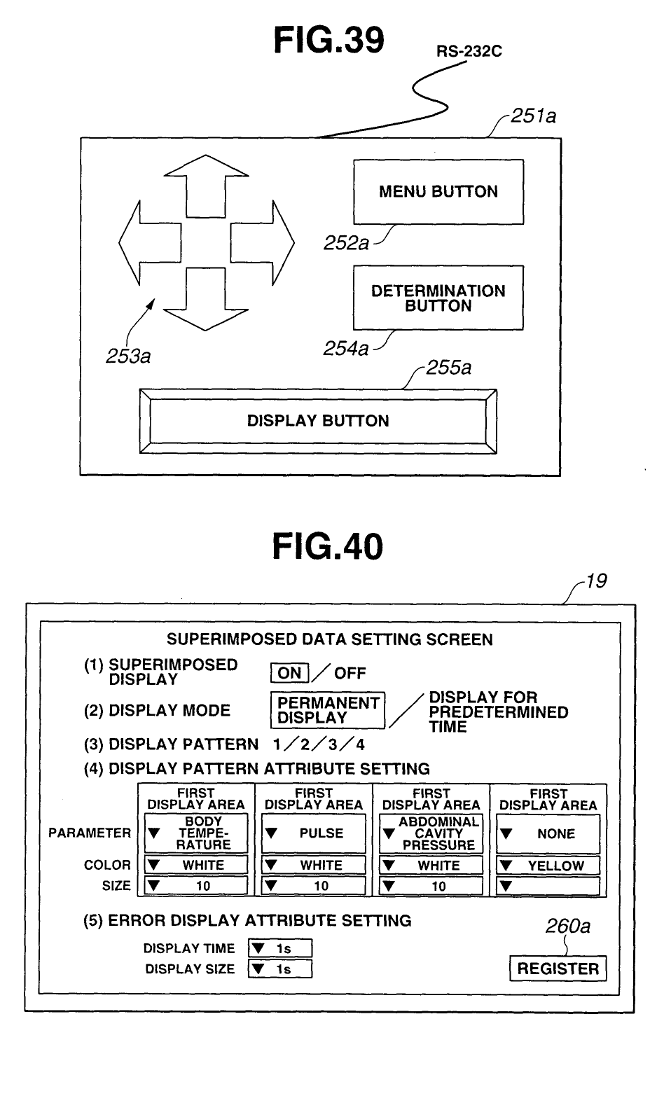

FIGS. 39 and 40 relate to a fourth embodiment of the present invention. FIG. 39 is a structural diagram which shows the construction of the remote controller, and FIG. 40 is a diagram which shows the superimposed data setting screen that is set and operated by the remote controller shown in FIG. 39.

The fourth embodiment is almost the same as the third embodiment. Accordingly, only points that are different will be described; the same constructions are labeled with the same symbols, and a description of such constructions is omitted.

[Construction]

In the present embodiment, a dedicated remote controller 251a of the type shown in FIG. 39 is provided instead of the remote controller 30. The remote controller 251a comprises a menu button 252a, a cursor key 253a, a determination button 254a, and a display button 255a.

Furthermore, it would also be possible to use a PDA with a touch sensor on the liquid crystal part instead of the remote controller 251a.

[Effect]

When the menu button 252a of the remote controller 251a is pressed, a superimposed data setting screen is displayed on the display device 19 as shown in FIG. 40. In this superimposed data setting screen, (1) the ON/OFF selection of superimposed display, (2) the selection of the display mode, (3) the selection of the display pattern, (4) the attribute setting of the display pattern, (5) the setting of error (warning) display attributes and the like are performed by operating the cursor key 253a and determination button 254a, and various types of settings of the superimposed data can be accomplished by checking the registration button 260a.

When this superimposed data setting screen is set, the display of the display device 19 shifts to the display of FIG. 35 described in the third embodiment. In the display of FIG. 35, for example, the remote controller 251a handles the respective inputs of display ON/OFF commands by the display button 255a, display pattern switching commands by the menu button 252a, and most-recent data display commands by the determination button 254a.

[Merits]

Thus, in the present embodiment, in addition to the merits of the third embodiment, the surgeon (user) can freely set attributes of the display pattern and the like; accordingly, the surgeon can check peripheral device state information and vital sign data in the desired positions, sizes, colors and the like.

As was described above, the third and fourth embodiments possess the following merits: specifically, various types of data can be superimposed on the endoscopic image in a desired display configuration, so that various types of data can be displayed without hindering observation of the image.

[Fifth Embodiment]

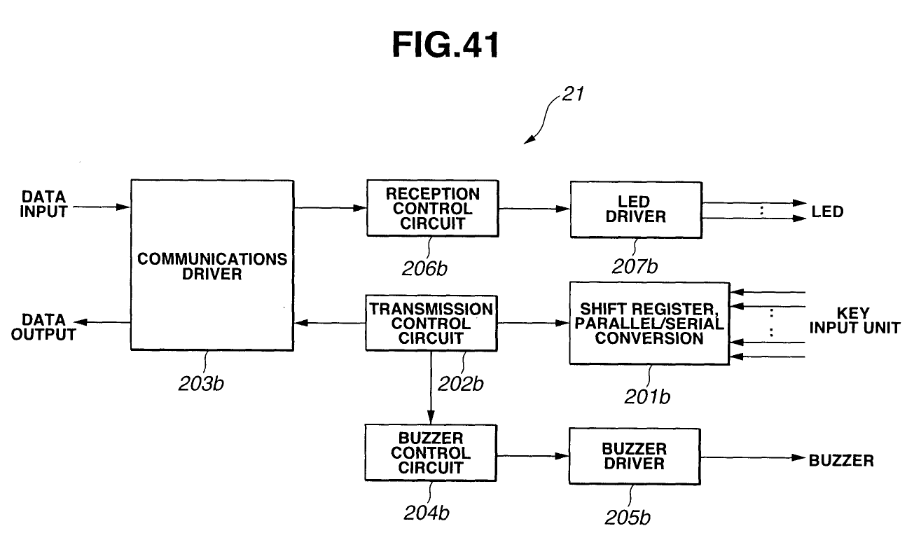

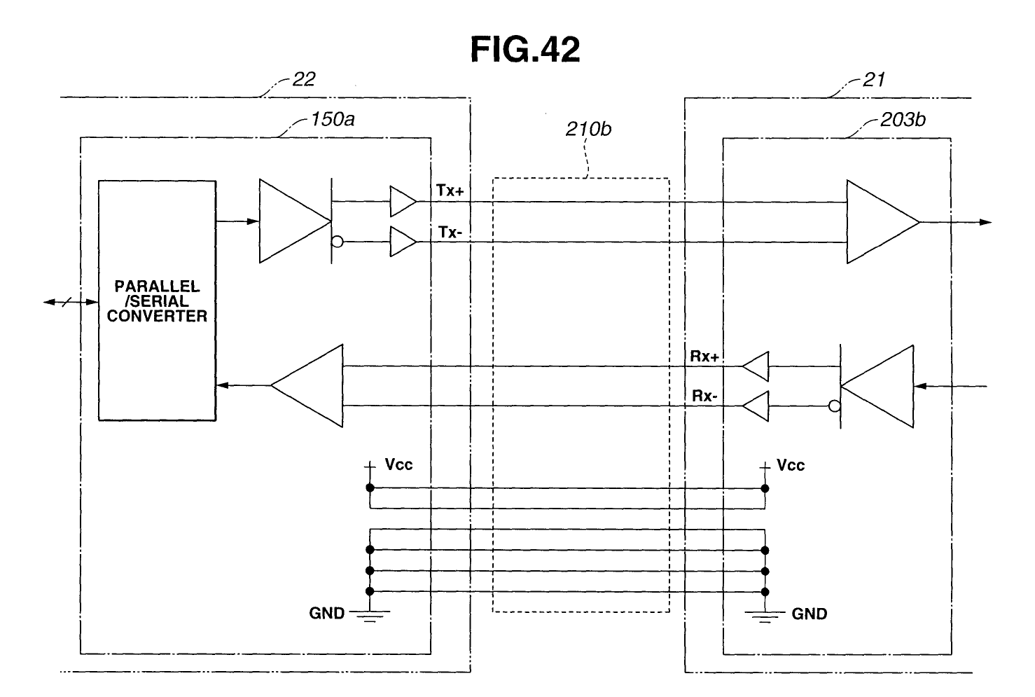

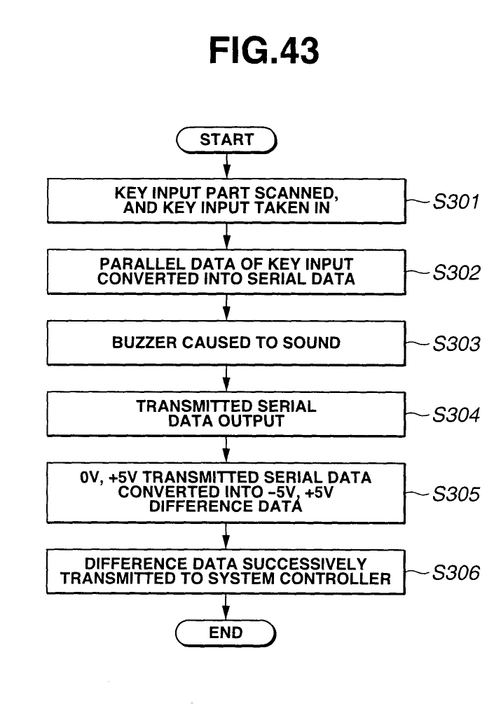

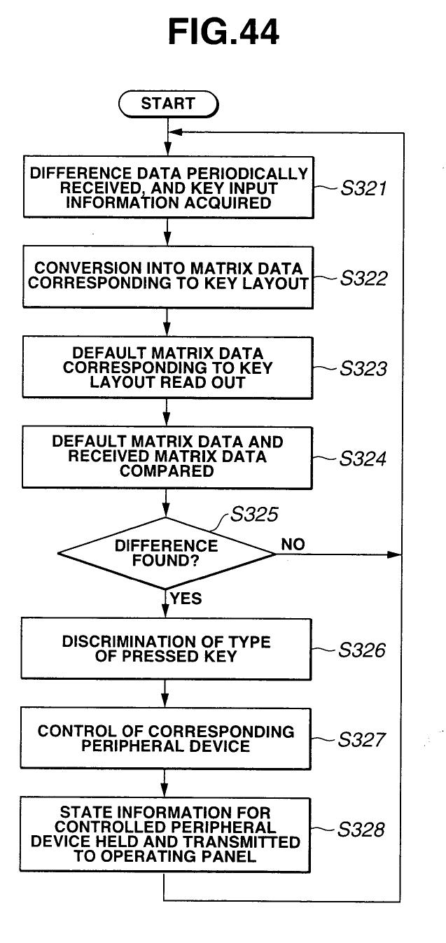

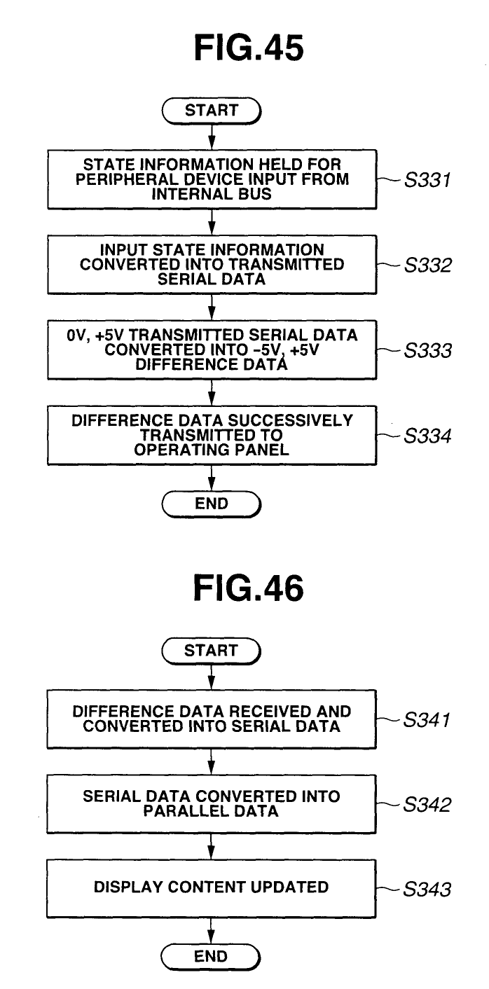

FIGS. 41 through 46 relate to a fifth embodiment of the present invention. Furthermore, since the overall construction of the system is similar to the constructions of the first through fourth embodiments, the same constituent elements are labeled with the same symbols, and a description of these constituent elements is omitted, in the description of the present embodiment. Accordingly, only items in the present embodiment that differ from the first and second embodiment will be described, and in particular, a description regarding FIGS. 1 through 6, FIG. 33 and FIG. 9 is omitted. FIG. 41 is a block diagram which shows the construction of the essential parts of the operating panel shown in FIG. 1, FIG. 42 is a diagram which shows the connection relationship between the system controller and operating panel in FIG. 1, FIG. 43 is a first flow chart which illustrates the operation of the endoscopic surgical system shown in FIG. 1, FIG. 44 is a second flow chart which illustrates the operation of the endoscopic surgical system shown in FIG. 1, FIG. 45 is a third flow chart which illustrates the operation of the endoscopic surgical system shown in FIG. 1, and FIG. 46 is a fourth flow chart which illustrates the operation of the endoscopic surgical system shown in FIG. 1.

[Construction]

The remote controller 30 is constructed as shown in FIG. 34, and is a second concentrated operating device that is operated by an operating surgeon in a sterile area. This device is arranged so that other devices with which communications have been established can be operated via the system controller 22.

In the fifth embodiment, as is shown in FIG. 5, a power supply switch 131, the abovementioned bidirectional infrared I/F 66 for the PDA 68, and the abovementioned unidirectional infrared I/F 67 for the infrared remote controller 69, are installed on the front surface of the system controller 22, and as is shown in FIG. 6, eight RS-232C communications connectors 135(1) through 135(8) (for example) which are used to control the electrical scalpel 13, insufflator 14, endoscopic camera device 15, light source device 16, VTR 17, concentrated display panel 20 and the like, an RS-422 communications connector 136 which is used to control the remote controller 30, a connector 137 such as a 10BaSe/T or the like which is used for connection with the hospital LAN 101, a BNC 138 which connects the display device 19, a pin jack 139 which transmits and receives video signals to and from the VTR 17, a communications connector 140 which is used for setting control of the operating panel 21 and the like are installed on the back surface of the system controller 22.

As is shown in FIG. 33, the system controller 22 comprises a character superimposing unit 151 which superimposes desired characters on the endoscopic image and outputs this image to the BNC 138, a setting operating unit I/F unit 152 which transmits and receives data to and from the operating panel 21, an infrared I/F unit 149 which performs infrared communications with the infrared remote controller 69 and PDA 68, a remote controller control I/F unit 153a which transmits and receives data to and from the remote controller 30, and a serial communications I/F unit 150a which performs serial communications via the RS-232C communications connectors 135(1) through 135(8) and RS-422 communications connector 136, and is constructed so that these parts are connected to an internal bus 154a.

A CPU 155a which controls the interior of the system controller 22 is connected to the abovementioned internal bus 154a, and the CPU 155a controls the interior of the system controller 22 using an EEPROM 156a, EEPROM 157a, RAM 158a and the like. Furthermore, a TCP/IP control unit 159a is connected to the CPU 155a, and a connection to the hospital LAN is made by the TCP/IP control unit 159a.

Furthermore, in the first and second embodiments, wireless communications are performed using infrared (unidirectional infrared communications and bidirectional infrared communications, e.g., an IrDA system or the like). However, electromagnetic wireless may also be used for the transmission and reception of peripheral device parameters in both directions; e.g., a wireless LAN, Bluetooth or the like may also be used. In this case, since wireless is used, communications can always be continued so that data can be exchanged without being blocked by obstacles.

As is shown in FIG. 9, the operating panel 21 is constructed from a display function, e.g., a plurality of seven-segment display devices and LEDs or the like, and a plurality of switches, and is a concentrated operating device which is operated by a nurse or the like in a non-sterile area.

Furthermore, as is shown in FIG. 41, the operating panel 21 comprises a shift register 201b which scans a key input unit comprising a plurality of switches, detects the key input states, and outputs these states as serial data, a transmission control circuit 202b which outputs the serial data from the shift register 201b as transmission serial data, and outputs a detection signal to a buzzer control circuit 204b when a key input is detected, a buzzer driver 205b which is controlled by the buzzer control circuit 204b, and which causes a buzzer to sound, a communications driver 203b which converts the transmission serial data from the transmission control circuit 202b into +5 V Rx+ and −5 V Rx− difference data, and outputs this data to the system controller 22, a reception control circuit 206b which receives (by means of the communications driver 203b) control commands comprising peripheral device state information and the like transmitted from the system controller 22 as +5 V Tx+ and −5V Tx− difference data, restores this data to reception serial data, and detects the control commands, and an LED driver 207b which drives the LEDs in accordance with the peripheral device state information under to control of the reception control circuit 206b.

As is shown in FIG. 42, the operating panel 21 and system controller 22 are connected by a communications cable 210b, i. e., the output connector of the operating panel 21 and (for example) the RS-422 communications connector 135 (see FIG. 6) of the system controller 22 are connected by the communications cable 210b. The setting operating unit I/F unit 152 which transmits and receives signals via the RS-422 communications connector 135 performs a parallel/serial conversion with respect to the internal bus 154a, creates +5 V Tx+ and −5 V Tx− difference data from the control commands comprising peripheral device state information and the like, and outputs this data to the operating panel 21. Furthermore, the I/F part 152 receives +5 V Rx+ and −5 V Rx− difference data from the operating panel 21, restores this to serial data, and outputs this data to the internal bus 154a.

[Effect]

In the operating panel 21, as is shown in FIG. 41, the key input unit is periodically scanned by the shift register 201b in step S301, and key input is taken in. In step S302, the parallel data of the key input is converted into serial data, and in step S303, the buzzer control circuit 204b controls the buzzer driver 205b so that the buzzer is caused to sound when a key input is detected by the transmission control circuit 202b. Furthermore, in step S304, the transmission control circuit 202b creates transmission serial data to which headers and the like have been added from the key input serial data, and outputs this data to the communications driver 203b. In step S305, the communications driver 203b converts the transmission serial data into +5 V Rx+ data and −5 V Rx− difference data, and in step S306, the communications driver 203b continuously outputs this data to the system controller 22.

In the system controller 22, as is shown in FIG. 44, the +5 V Rx+ and −5 V Rx− difference data from the operating panel 21 is periodically received in step S321, and the headers are detected and converted into serial data. Then, the data is converted into parallel data and output via the internal bus 154a, so that the CPU 155a acquires the key input information.

In step S322, the CPU 155a converts the acquired key input information into matrix data corresponding to the key layout, and in step S323, the default matrix data corresponding to the key layout which has been stored in the EEPROM 157a beforehand is read out. Then, the acquired matrix data and the default matrix data are compared in step S324.

When it is determined in step S325 (as a result of the comparison) that there is a difference from the default matrix data, the type of key pressed is discriminated from the elements of the different matrix data in step S326, and the peripheral device corresponding to the key input is controlled in step S327. Then, in step S328, the state information of the controlled peripheral device is held, and the state information of the peripheral device is transmitted to the operating panel 21.

As is shown in FIG. 45, the details of step S328 are as follows: specifically, in the setting operating unit I/F unit 152 of the system controller 22, when the peripheral device state information that is held is input from the internal bus 154a in step S331, the state information is converted into serial data in step S332, the serial data is further converted into +5 V Tx+ and −5 V Tx− difference data in step S333, and this data is output to the operating panel 21 via the communications cable 210b in step S334.

Furthermore, in the operating panel 21, as is shown in FIG. 46, the +5 V Tx+ and −5 V Tx− difference data from the system controller 22 is received by the communications driver 203b and restored to reception serial data in step S341. Then, in step S342, the peripheral device information is recognized in the reception control circuit 206b, the reception serial data is converted into parallel data, and this data is output to the LED driver 207b, so that the LED driver 207b updates the display content in step S343.

[Merits]

Thus, in the present embodiment, the operating panel 21 and system controller 22 can perform communications without a handshake; accordingly, as is shown in FIG. 40, the number of signal lines of the communications cable 210b used for communications can be reduced, and the signal lines that are not used for communications can be assigned to GND lines. As a result, stable communications can be ensured, and power can be supplied to the concentrated control panel via the communications cable; accordingly, connections may be established using the communications cable alone without any need for a power supply cable, so that the size of the apparatus can be reduced.

As was described above, the fifth embodiment possesses the following merits: specifically, stable communications can be ensured, power can be supplied to the concentrated control panel via the communications cable, and the size of the apparatus can be reduced.

[Sixth Embodiment]

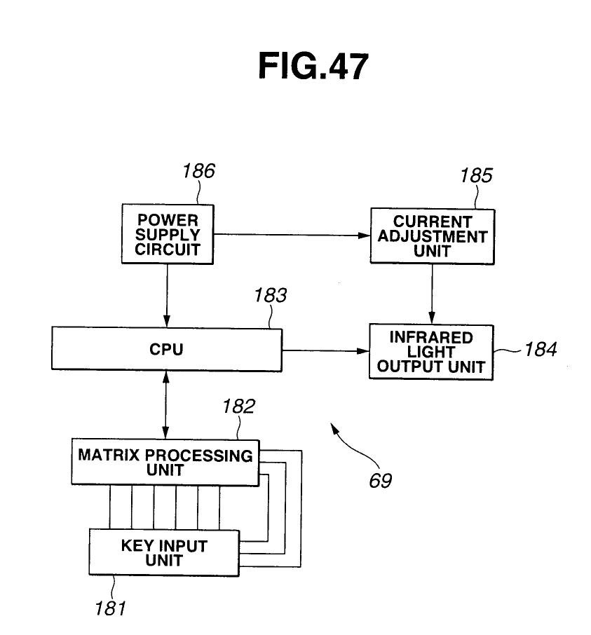

FIGS. 47 through 64 relate to a sixth embodiment of the present invention. Furthermore, since the overall construction of the system is similar to the constructions of the first through fifth embodiments, the same constituent elements are labeled with the same symbols, and a description of such elements is omitted, in the description of the present embodiment. Accordingly, only items of the present embodiment that differ from the first and second embodiments will be described, and in particular, a description regarding FIGS. 1 through FIG. 6, FIG. 8, FIGS. 10 through 12, FIG. 13 and FIG. 33 will be omitted.

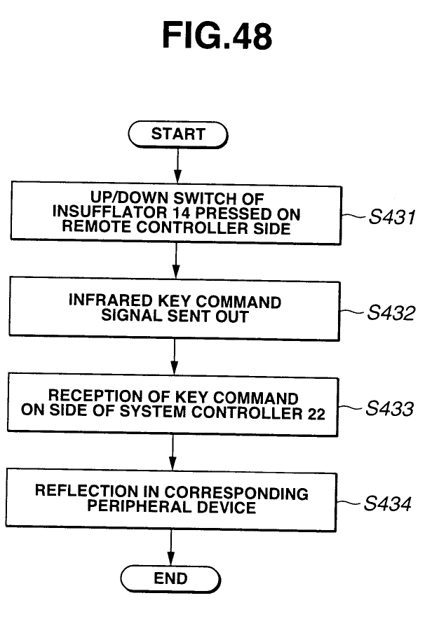

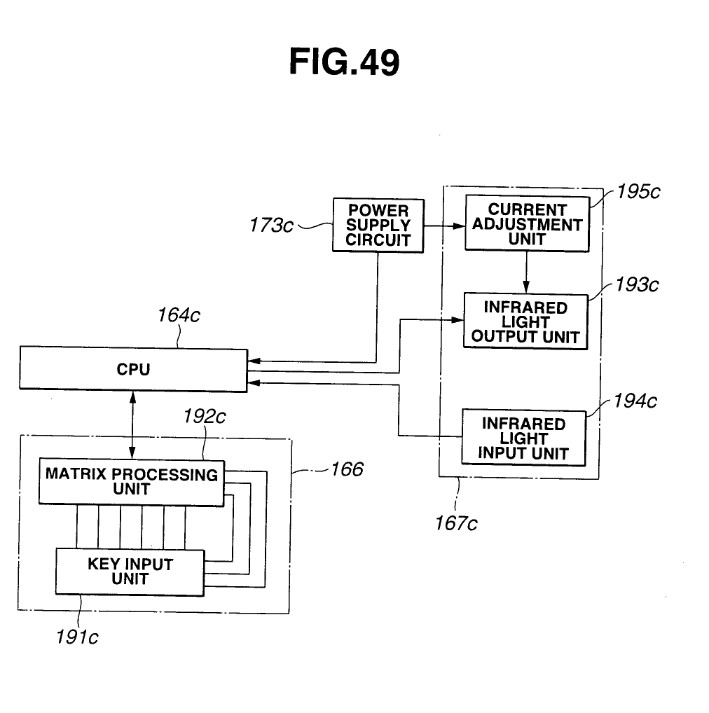

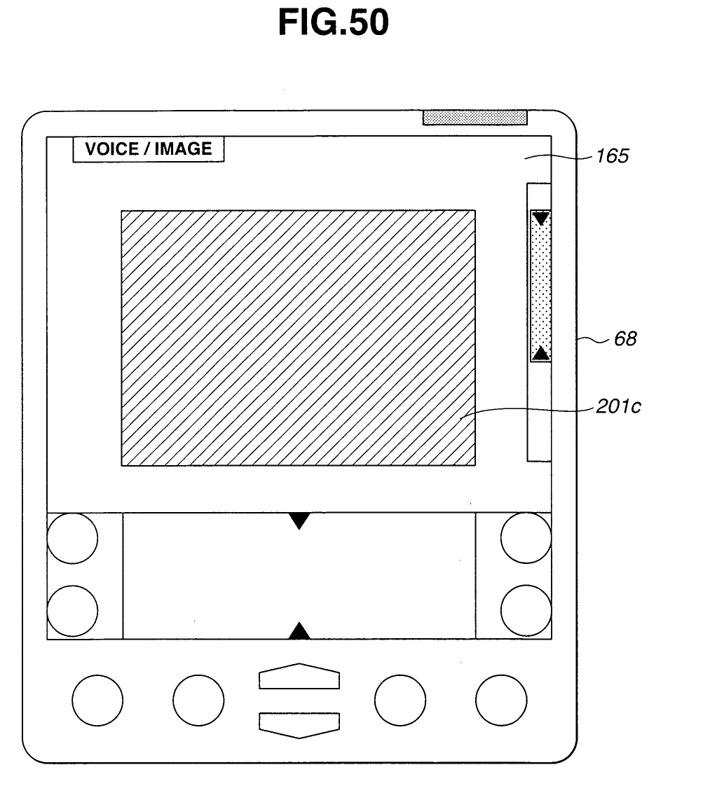

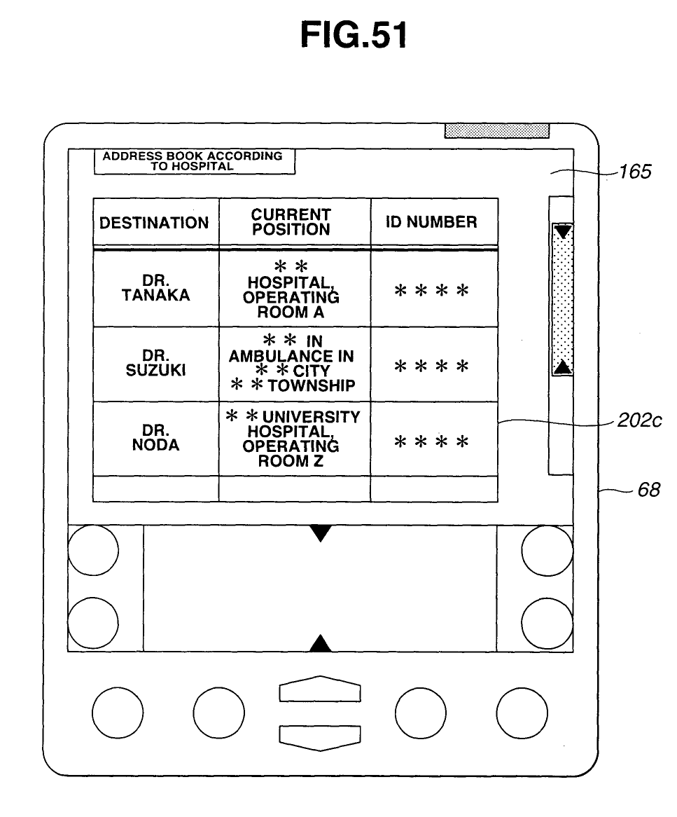

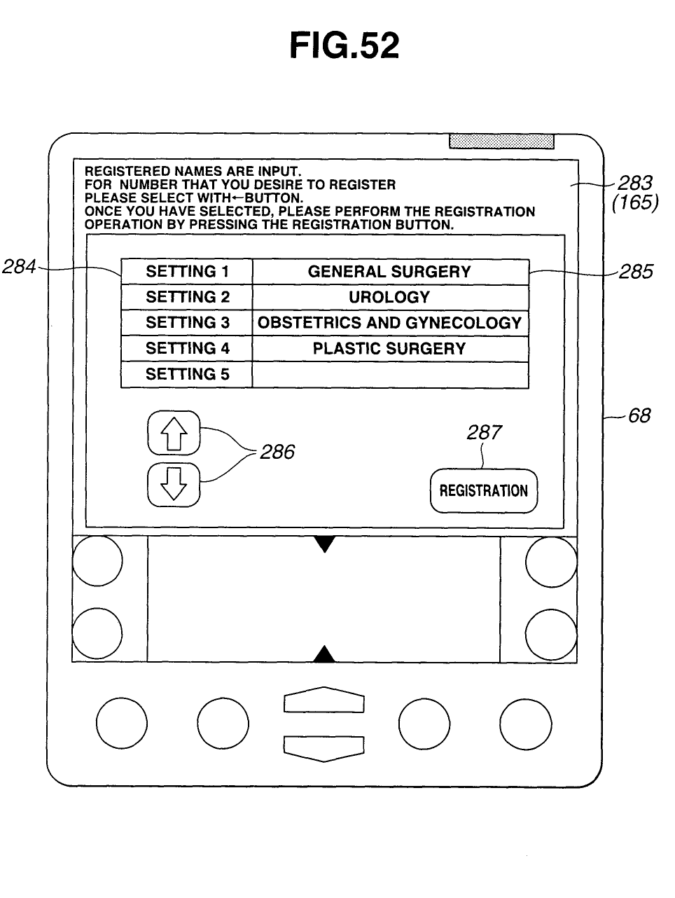

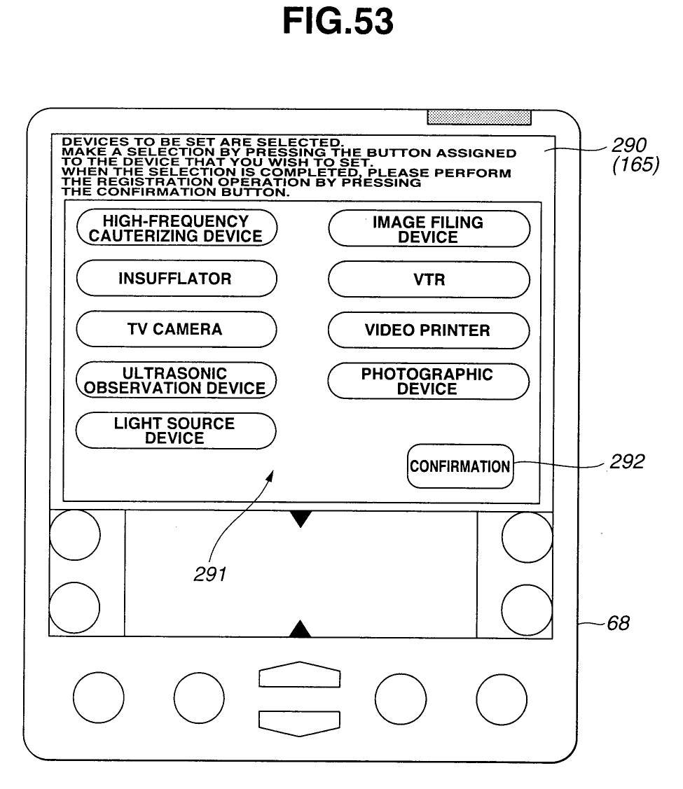

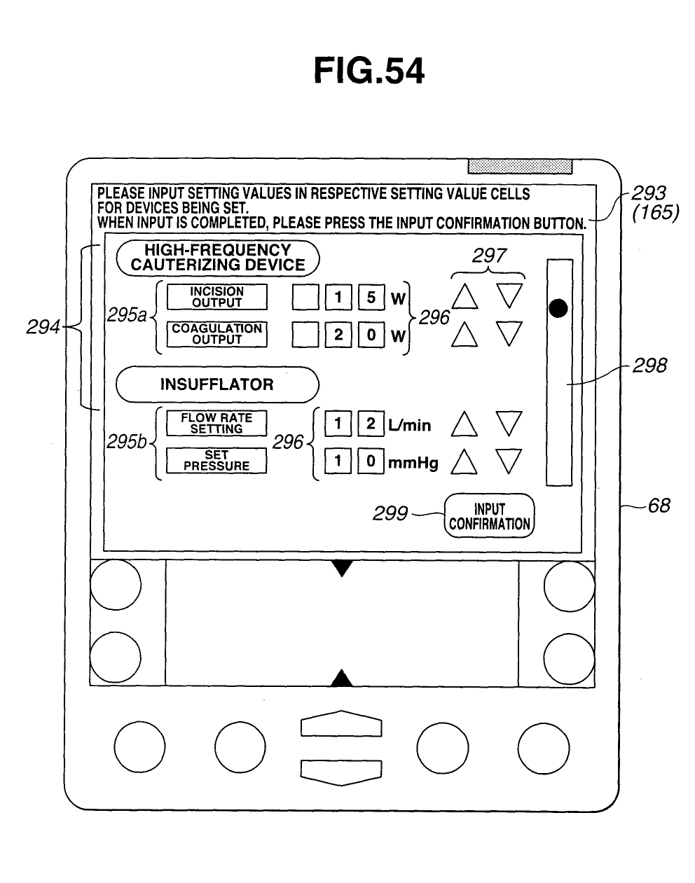

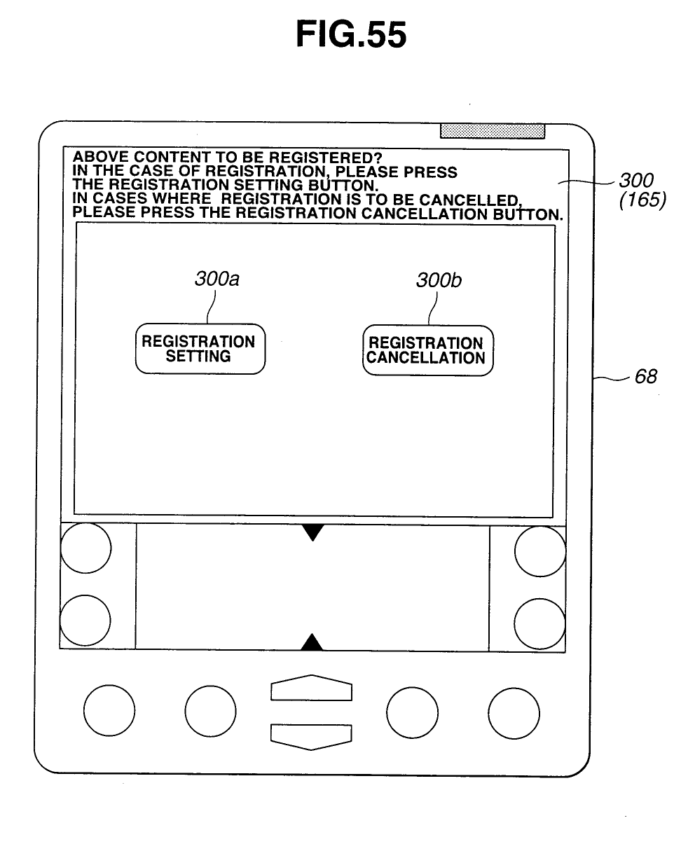

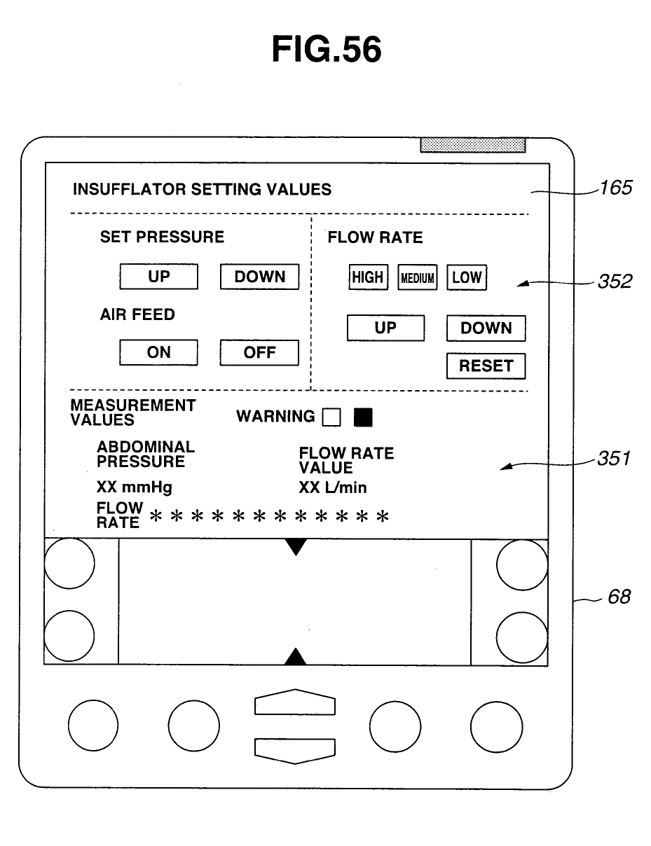

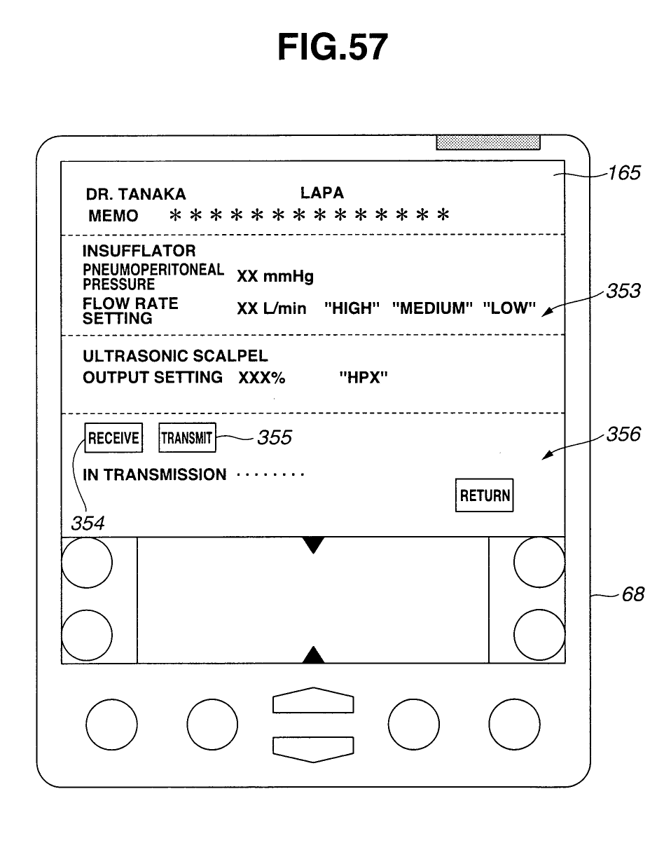

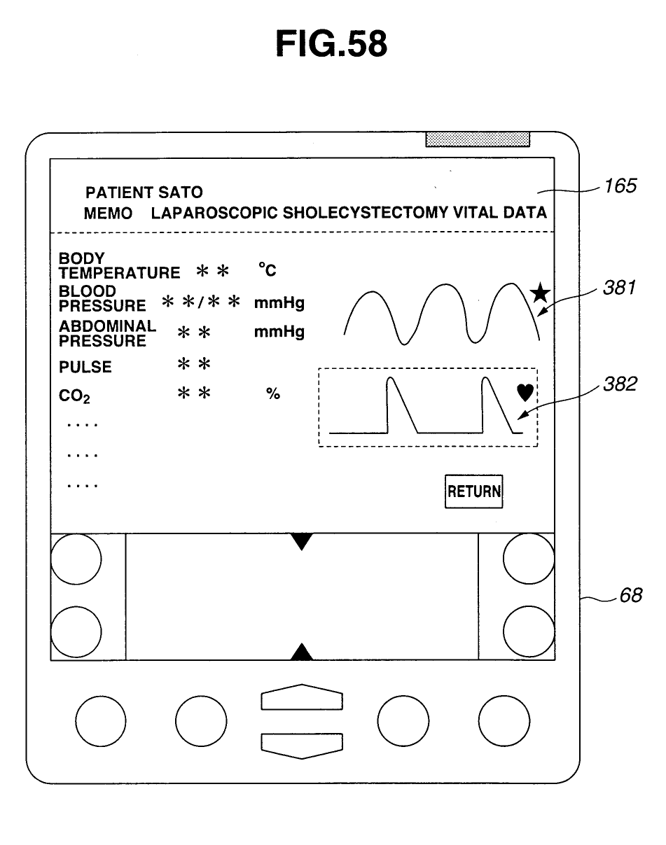

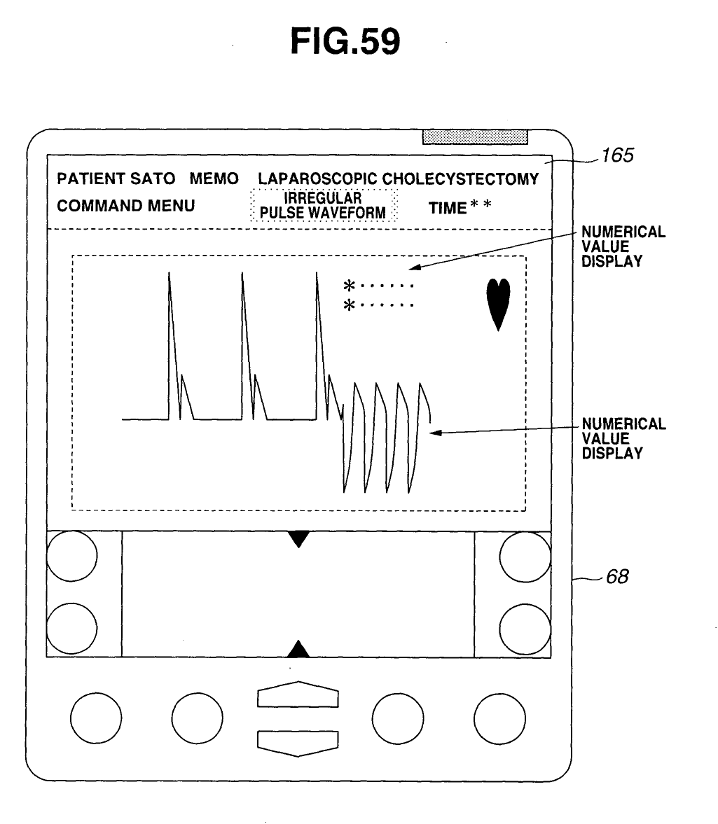

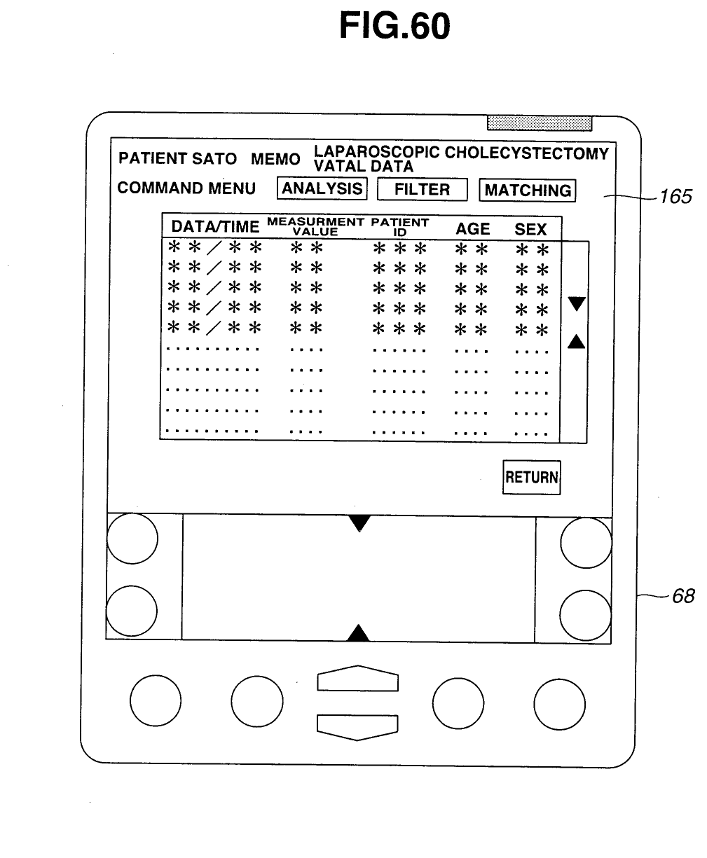

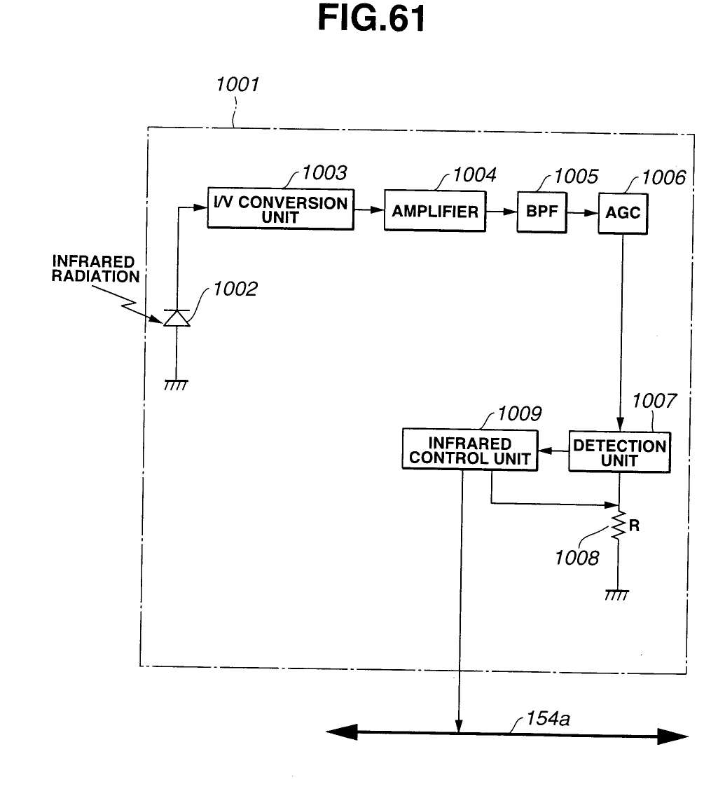

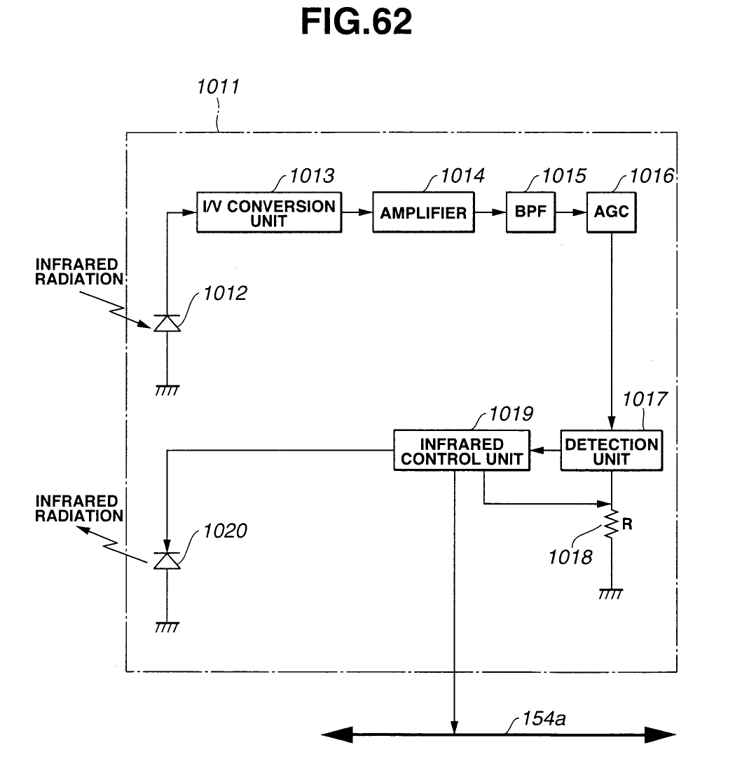

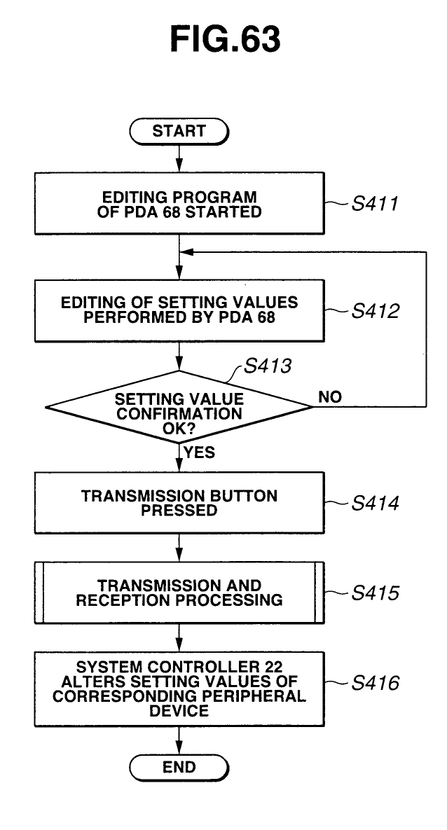

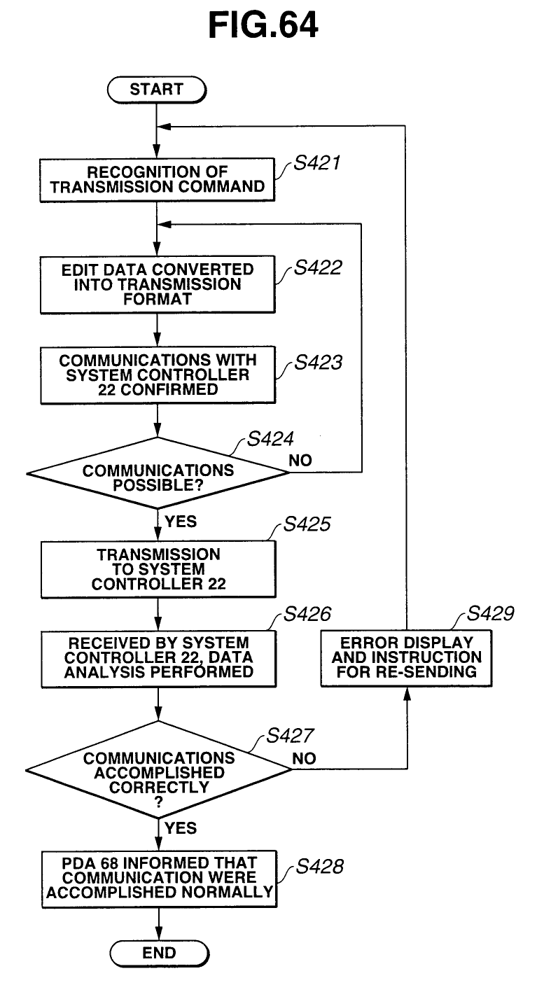

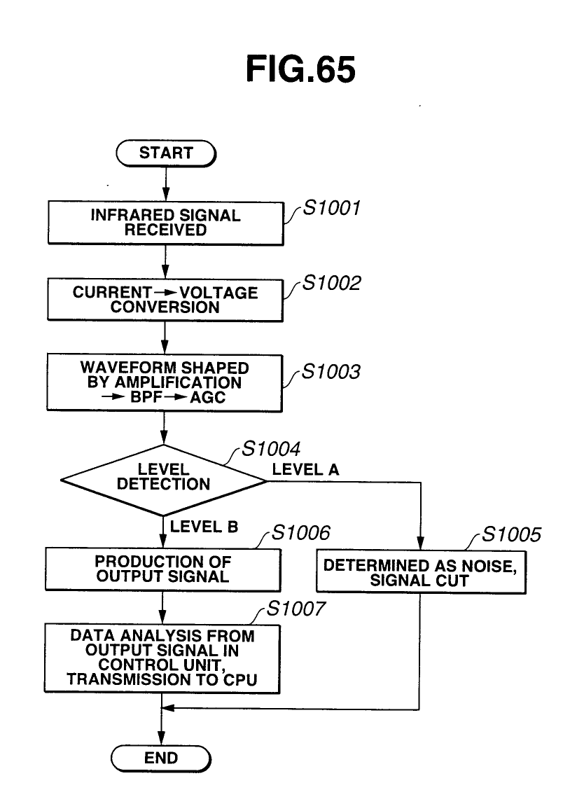

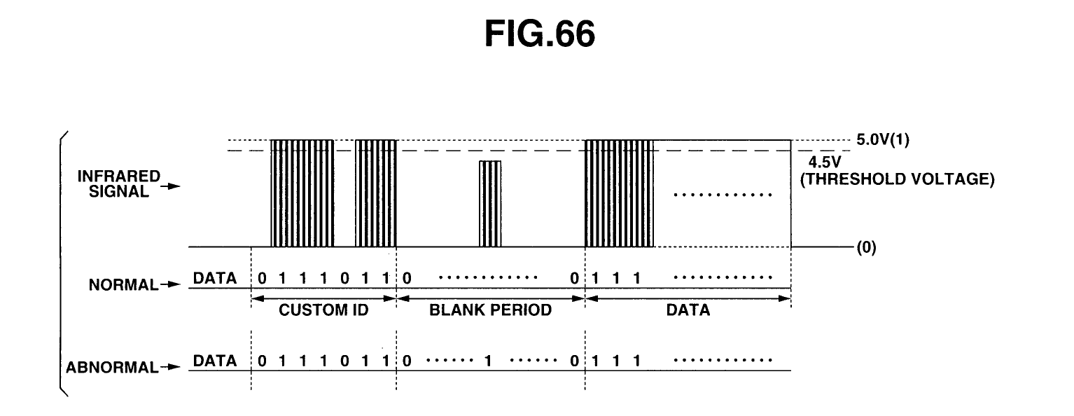

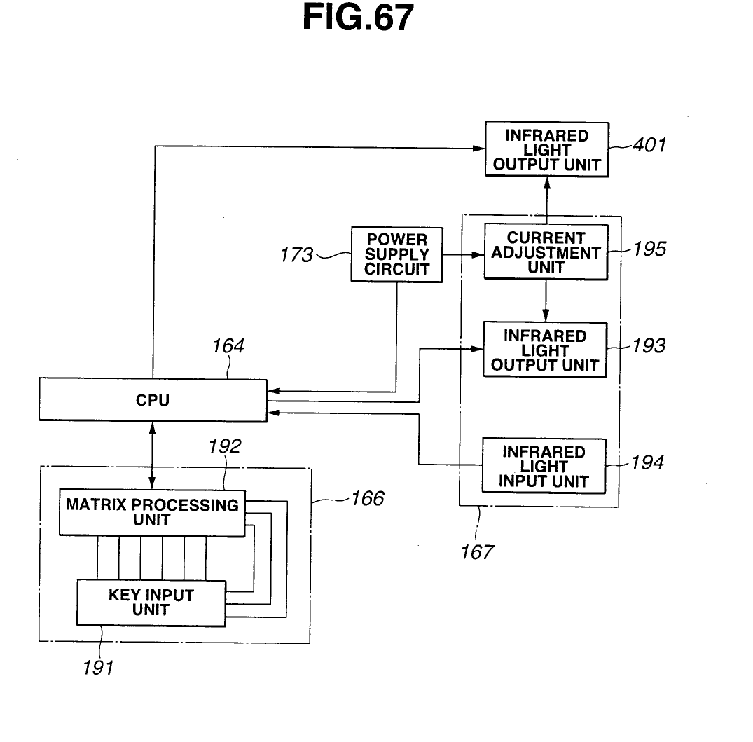

FIG. 47 is a block diagram which shows the construction of the infrared remote controller shown in FIG. 1, FIG. 48 is a flow chart which shows the flow of the processing that is performed when a peripheral device is operated by the unidirectional infrared remote controller shown in FIG. 1, FIG. 49 is a block diagram which shows the construction of the touch panel and wireless communications interface (I/F) shown in FIG. 8, FIG. 50 is a diagram which shows a second screen displayed by the liquid crystal display shown in FIG. 8, FIG. 51 is a diagram which shows a third screen displayed by the liquid crystal display part shown in FIG. 8, FIG. 52 is a diagram which shows a fourth screen displayed by the liquid crystal display part shown in FIG. 8, FIG. 53 is a diagram which shows a fifth screen displayed by the liquid crystal display part shown in FIG. 8, FIG. 54 is a diagram which shows a sixth screen displayed by the liquid crystal display part shown in FIG. 8, FIG. 55 is a diagram which shows a seventh screen displayed by the liquid crystal display part shown in FIG. 8, FIG. 56 is a diagram which shows an eighth screen displayed by the liquid crystal display part shown in FIG. 8, FIG. 57 is a diagram which shows a ninth screen displayed by the liquid crystal display part shown in FIG. 8, FIG. 58 is a diagram which shows a tenth screen displayed by the liquid crystal display part shown in FIG. 8, FIG. 59 is a diagram which shows an eleventh screen displayed by the liquid crystal display part shown in FIG. 8, FIG. 60 is a diagram which shows a twelfth screen displayed by the liquid crystal display part shown in FIG. 8, FIG. 61 is a diagram which shows the construction of the unidirectional infrared communications controller of the unidirectional infrared communications interface (I/F) shown in FIG. 1, FIG. 62 is a diagram which shows the construction of the bidirectional infrared communications controller of the bidirectional infrared communications interface (I/F) shown in FIG. 1, FIG. 63 is a first flow chart which shows the flow of the processing that is performed when a peripheral device is operated by the PDA shown in FIG. 1, FIG. 64 is a second flow chart which shows the flow of the processing that is performed when a peripheral device is operated by the PDA shown in FIG. 1, FIG. 65 is a flow chart which illustrates the operation of the unidirectional infrared communications controller and bidirectional infrared communications controller shown in FIG. 61 and FIG. 62, and FIG. 66 is a diagram which is used to describe the flow chart shown in FIG. 65.

The remote controller 30 is a second concentrated operating device that is operated by an operating surgeon in a sterile area. This device is arranged so that other devices with which communications have been established can be operated via the system controller 22.

As is shown in FIG. 33, the system controller 22 comprises a character superimposing unit 151 which superimposes desired characters on the endoscopic image and outputs this image to the BNC 138, a setting operating unit I/F unit 152 which transmits and receives data to and from the operating panel 21, an infrared I/F unit 149 which performs infrared communications with the infrared remote controller 69 and PDA 68, a remote controller control I/F unit 153a which transmits and receives data to and from the remote controller 30, and a serial communications I/F unit 150a which performs serial communications via the RS-232C communications connectors 135(1) through 135(8) and the RS-422 communications connector 136, and is constructed so that these parts are connected to the internal bus 154a.

Furthermore, a CPU 155a which controls the interior of the system controller 22 is connected to the abovementioned internal bus 154a, and the CPU 155a controls the interior of the system controller 22 using an EEPROM 156a, EEPROM 157a, RAM 158a and the like. Furthermore, a TCP/IP control unit 159a is connected to the CPU 155a, and a connection to the hospital LAN is made by the TCP/IP control unit 159a.

As is shown in FIG. 5, a power supply switch 131, the abovementioned unidirectional infrared I/F 67 for the infrared remote controller 69, and the abovementioned bidirectional infrared I/F 66 for the PDA 68, are installed on the front surface of the system controller 22, and as is shown in FIG. 6, eight RS-232C communications connectors 135(1) through 135(8) (for example) which are used to control the electrical scalpel 13, insufflator 14, endoscopic camera device 15, light source device 16, VTR 17, concentrated display panel 20, and the like, an RS-422 communications connector 136 which is used to control the remote controller 30, a 10BaSe/T connector 137 which is used for connection with the hospital LAN 101, a BNC 138 which connects the display device 19, a pin jack 139 which transmits and receives video signals to and from the VTR 17, a communications connector 140 which is used for setting control of the operating panel 21 and the like are installed on the back surface of the system controller 22.

As is shown in FIG. 47, the infrared remote controller 69 is constructed from a key input unit 181 comprising a plurality of key switches, a matrix processing unit 182 which scans the key input unit 181, a CPU 183 which produces key codes corresponding to the key inputs of the key input unit 181, an infrared output unit 184 which outputs infrared pulses corresponding to the key codes to the system controller 22, and performs unidirectional communications, a current adjustment unit which adjusts the driving current of the infrared output unit 184, and a power supply circuit 186 which supplies power to the CPU 183 and current adjustment unit 185. The key layout of the key input unit 181 of the infrared remote controller 69 is as shown in FIG. 13.

Furthermore, in the present embodiment, wireless communications are performed using infrared (unidirectional infrared communications and bidirectional infrared communications, e.g., an IrDA system or the like). However, electromagnetic wireless may also be used for the transmission and reception of peripheral device parameters in both directions; e.g., a wireless LAN, Bluetooth or the like may also be used. In this case, since wireless is used, communications can always be continued so that data can be exchanged without being blocked by obstacles.

Furthermore, FIG. 48 shows a flow chart of the flow in a case where the operation of peripheral devices is performed by TV remote controller using unidirectional infrared communications. The detailed flow of the processing will be described later.

As is shown in FIG. 49, the touch panel 166 of the PDA 68 is constructed from a key input unit 191c comprising touch sensors formed in a matrix pattern, and a matrix processing unit 192c which scans the key input unit 191c. Furthermore, the wireless communications I/F part 167c is constructed from an infrared output unit 193c which outputs infrared pulses corresponding to command codes (produced by the CPU 164c in accordance with the key inputs of the key input unit 191c) to the system controller 22, an infrared input unit 194c which inputs infrared pulses from the system controller 22 and outputs these pulses to the CPU 164c, and a current adjustment unit 195c which adjusts the driving current of the infrared output unit 193c.

As is shown in FIG. 10, a liquid crystal display part 165 on which a touch panel 166 is installed is disposed on the front surface of the PDA 68, and a portion of the liquid crystal display part 165 constitutes an handwritten input part. Furthermore, as is shown in FIG. 11, a card slot 169 and an external communications I/F 171 are disposed on the back surface of the PDA 68. Examples of expansion cards 168 that can be mounted in the card slot 169 include movie image communications expansion cards, still image communications expansion cards, GPS (global positioning system) expansion cards, modem expansion cards and the like such as those shown in FIG. 12.

Data can be exchanged with the system controller 22 by IrDA communications by touching the touch panel 166 on the menu screen of the liquid crystal display part 165 shown in FIG. 10 with the fingers or with a stylus pen or the like. For example, an endoscopic image 201 such as that shown in FIG. 50 can be displayed on the liquid crystal display part 165. Furthermore, if users such as a doctor or the like who have a PDA in which a GPS expansion card constituting an expansion card 168 is mounted in the card slot 169 are ready to make access to the internet, addresses of those users can be displayed on the liquid crystal display part 165 as an address book 202c as shown in FIG. 51.

Furthermore, a registration item button (not shown in the figures) which is used to register setting values is disposed on the menu screen of the liquid crystal display part 165 shown in FIG. 10, and when a user operates the registration item button by operating the touch panel 166, the screen on the liquid crystal display part 165 is switched to the registered name input image 283 shown in FIG. 52.

The registered name input image 283 shown in FIG. 52 is an image which is used to input registered names for the respective operating rooms 2 described in FIG. 1, in accordance with the type of surgery or the like. Registered name input cells 285 into which registered names are input is disposed on the right side of the setting number cells 284. An up-down button 286 which is used to move the cursor between the respective registered name input cells 285 is disposed beneath the setting number cells 284. Furthermore, a registration button 287 is disposed on the lower right portion of the screen.

The user inputs registered names into the PDA 68 using the touch panel 166. Here, in FIG. 52, a case is shown in which registered names have already been input into the registered name cells 285 from “setting 1” to “setting 4”, and the cursor is positioned at “setting 5”, so that registered name can be input into the registered name input cell 285 of the “setting 5”.

Furthermore, in regard to the registered names that are input into the registered name input cells 285, for example, “setting 1” is general surgery, “setting 2” is urology, “setting 3” is obstetrics and gynecology, and “setting 4” is plastic surgery. Furthermore, in FIG. 52, the registered name input image 283 is disposed from “setting 1” to “setting 5”; however, further settings can be accomplished by scrolling the display cells with the movement of the cursor.

Furthermore, registered names are registered by the user operating the registration button 287 by similarly operating the touch panel 166 after the registered names are input. As a result, the registered names are set (stored in memory) in the PDA 68, and registered names corresponding to types of surgery or the like can be assigned by exchanging data with the system controller 22 by IrDA communications. Accordingly, by selecting registered names that have been registered, the user can make selections and settings which are such that the respective medical devices disposed in the operating room 2 have the desired settings. Furthermore, when the registration button 287 is operated, the screen on the liquid crystal display device 165 is switched to the device selection image 290 shown in FIG. 53.

The device selection image 290 shown in FIG. 53 is an image which is used to select medical devices for which registration is desired on the screen. On the device selection image 290, the names of the high-frequency cauterizing device and the like are disposed in medical device display cells 291. Furthermore, a confirmation button 292 is disposed on the lower right portion of the screen.

Here, the user uses the touch panel 166 to select medical devices for which registration is desired, and confirms these devices by operating the confirmation button 292.

Furthermore, in the present embodiment, the high-frequency cauterizing device and insufflator have been selected as medical devices. Then, when the confirmation button 292 is operated, the screen on the liquid crystal display device 165 is switched to the setting input image 293 shown in FIG. 54.

The setting input image 293 shown in FIG. 54 is an image which is used to perform setting input for medical devices selected by the device selection screen 290 illustrated in FIG. 53. The setting input screen 293 is arranged so that desired setting values can be input for the medical devices selected by the user in FIG. 53. In the setting input screen 293, respective treatment mode name cells 295a and setting name cells 295b are disposed beneath peripheral device name display cells 294, and setting value input cells 296 are disposed in adjacent positions to the right of these respective name cells 295a and 295b.

Up-down buttons 297 which are used to move the setting values that are input into the setting value input cells 296 upward or downward are disposed in adjacent positions to the right of these setting value input cells 296.

Furthermore, a list display cell 298 which displays the position of the selected setting value input cell 296 when one of the setting value input cells 296 is selected is disposed in an adjacent position to the right of the up-down buttons 297. Furthermore, an input confirmation button 299 which confirms the input of the setting value input cells 296 is disposed beneath the up-down buttons 297.

Here, the user uses the touch panel 166 to input desired setting values in to the setting value input cells 296 for the selected medical devices. When input is completed, this input is confirmed by operating the input confirmation button 299. Then, when the input confirmation button 299 is operated, the screen on the liquid crystal display device 165 is switched to the registration confirmation image 300 shown in FIG. 55.

The registration confirmation image 300 shown in FIG. 55 is an image which is used to confirm the registration of the content registered by the operation up to the setting input image 293 illustrated in FIG. 54. In the registration confirmation image 300, a registration confirmation button 300a which is used to confirm the registration of the registered content, and a registration cancellation button 300b which is used to cancel the registration of the registered content, are disposed side by side in the center of the screen.

When the registered content is satisfactory, the user completes the registration by sing the touch panel 166 to operate the registration confirmation button 300a. Then, when the registration confirmation button 300a is operated, the screen on the liquid crystal display part 165 is switched to the menu screen shown in FIG. 10.

On the other hand, in case where the user is not satisfied with the registered content, the user uses the touch panel 166 to operate the registration cancellation button 300b, and repeats the registration operation until satisfied with the registered content. Here, when the registration cancellation button 300b is operated, the screen on the liquid crystal display device 165 is switched to the registered name input image 283 illustrated in FIG. 52.

Furthermore, in the PDA 68, the states of the respective medical devices disposed in the operating room 2 can be downloaded and displayed on the liquid crystal display device 165 by exchanging data with the system controller 22 by IrDA communications. For example, a measurement value screen 351 showing the abdominal cavity pressure, flow rate and the like of the insufflator 14 (as shown in FIG. 56) can be displayed on the liquid crystal display device 165. In this case, the settings can be altered by displaying the setting screen 352 used to input setting values on the liquid crystal display device 165.

When the touch panel 166 is operated in the setting screen 352, the screen shifts to a data transmission screen 353 such as that shown in FIG. 57. The setting data for respective medical devices set by the PDA 68 can be transmitted to the system controller 22 by IrDA communications by pressing the transmission button 354. Furthermore, state information for the respective medical devices disposed in the operating room 2 can be received from the system controller 22 by IrDA communications by pressing the reception button 355.

For example, when vital sign data under a laparoscopic cholecystectomy being monitored by the patient monitoring system 4 is received from the system controller 22 by IrDA communications, data such as the body temperature, blood pressure, pulse and the like of the patient, as well as (for example) a blood pressure waveform diagram 381 and electrocardiogram 382, can be displayed on the liquid crystal display device 165 in the PDA 68 as shown in FIG. 58. Furthermore, for example, if the electrocardiogram 382 is selected by the touch panel 166, the electrocardiogram 382 can be displayed in enlarged form as shown in FIG. 59. Furthermore, if an area of interest such as an abnormal waveform or the like is detected in this enlarged electrocardiogram, data for the area of interest can be converted into numerical values and displayed by pressing the area of interest with the touch panel 166.

Furthermore, the system is arranged so that when the electrocardiogram 382 is selected by the touch panel 166, the electrocardiogram 382 is displayed in enlarged form. However, the present invention is not limited to this; for example, it would also be possible to display numerical data for the pulse waveform on the liquid crystal display device 165.

Thus, in the system controller 22 of the present embodiment, respective device control commands for a plurality of keys are assigned on the side of the infrared remote controller 69 using a device such as a TV remote controller utilizing infrared radiation as the infrared remote controller 69, so that the response speed from the unidirectional transmission of the key codes by infrared radiation and reception processing by the system controller 22 up to the updating in respective devices is increased. Furthermore, in the case of numerical data such as device measurement data, patient information and the like, this numerical data is transmitted and received using a device such as the PDA 68, which is a portable terminal capable of bidirectional communications.

Here, the respective I/Fs shown in FIG. 33 are constructed using a device known as an FPGA (field programmable gate array).

Next, the parts of the infrared I/F 149 will be described with reference to FIGS. 61 and 62. The infrared I/F 149 is comprised of the abovementioned bidirectional infrared communications I/F 66 and unidirectional infrared communications I/F 67. A driver and a controller are respectively comprised in each I/F; FIG. 61 shows the detailed construction of the unidirectional infrared communications controller 1001 in the unidirectional infrared communications I/F 67.

As is shown in FIG. 61, the unidirectional infrared communications controller 1001 comprises an infrared light receiving element 1002, an I/V conversion unit 1003 which converts the current produced by photoelectric conversion by the light receiving element 1002 into a voltage, a signal amplifier 1004 which amplifies the output of the I/V conversion unit 1003, a BPF (band pass filter) 1005 which passes only a certain frequency band of the signal amplified by the signal amplifier 1004, and which has upper-limit and lower-limit frequencies (fH and fL) of this frequency band, and an AGC (auto-gain control) 1006 which automatically adjusts the strength of the signal according to distance.

For example, the AGC 1006 automatically adjusts the reception sensitivity in cases where the distance is great so that the infrared signal has become weak, and has the function of automatically adjusting the communications sensitivity to the optimal sensitivity.

Furthermore, the unidirectional infrared communications controller 1001 is equipped with a detection unit 1007 which is used to extract only a specified signal from the signal whose gain has been controlled, and a resistance R 1008 which is used for a reference voltage is connected to the detection unit 1007. The signal detected by the detection unit 1007 is output to an infrared control unit 1009. The infrared control unit 1009 is connected to the CPU 155a via the internal bus 154a.

FIG. 62 shows the detailed construction of the bidirectional infrared communications controller 1011 in the bidirectional infrared communications I/F 66.

As is shown in FIG. 62, the bidirectional infrared communications controller 1011 comprises an infrared light receiving element 1012, an I/V conversion unit 1013 which converts the current produced by photoelectric conversion by the light receiving element 1012 into a voltage, a signal amplifier 1014 which amplifies the output of the I/V conversion unit 1013, a BPF (band pass filter) 1015 which passes only a certain frequency band of the signal amplified by the signal amplifier 1014, and which has upper-limit and lower-limit frequencies (fH and fL) of this frequency band, and an AGC (auto-gain control) 1016 which automatically adjusts the strength of the signal according to distance.

Furthermore, the bidirectional infrared communications controller 1011 is equipped with a detection unit 1017 which is used to extract only a specified signal from the signal whose gain has been controlled, and a resistance R 1018 which is used for a reference voltage is connected to the detection unit 1017. The signal detected by the detection unit 1017 is output to an infrared control unit 1019. The infrared control unit 1019 is connected to the CPU 155a via the internal bus 154a. Furthermore, the infrared control unit 1019 drives and controls a light-emitting element 1020, and the light-emitting element 1020 transmits infrared signals.

[Effect]

The operation of the PDA 68 in a case where the abovementioned construction is used will be described with reference to FIGS. 63 and 64. Furthermore, the operation of the unidirectional infrared remote controller 69 will be described with reference to FIG. 48.

In the flow chart shown in FIG. 63, the parameter editing program is started from the menu icon of the PDA 68 shown in FIG. 10 in step S411. In step S412, the parameters of the peripheral device for which remote controller is desired (the parameters shown in FIG. 59 or the like) are altered. This operation means that the operator edits the setting values, and data is stored in a predetermined register of the memory of the PDA 68. When the edited content is OK in step S413, the transmission button is pressed in step S414. In step S415, bidirectional communications are performed between the system controller 22 and the PDA 68.

The flow of the transmission operation in bidirectional communications will be described with reference to the flow chart shown in FIG. 64.

In step S421, the transmission command of the PDA 68 is recognized, and in step S422, the edited data is read out from the memory, and converted into a transmittable format. For example, packet communications (a system that performs communications using a data structure having individual IDs or port numbers) or the like may be used. In the present embodiment, the transmission data, the type of the data, the version of the communications protocol, read/write and the like are transmitted and received as a single data structure. The “type of data” is information for the peripheral device for which updating is desired, and refers to an ID number. Furthermore, the data may be numerical data of peripheral device parameters, or a plurality of sets of data such as ON/OFF information or the like may be used.

In step S423, the PDA 68 sends a communications request to the system controller 22, and is placed in a state that allows communications. When a state that allows communications is established in step S424, data is transmitted to the system controller 22 in step S425. In step S426, the system controller 22 analyzes the communications content on the basis of the abovementioned data type and version information. When communications are correctly accomplished in step S427 from the analysis results of step S426, then the PDA 68 is informed in step S428 that communications have been performed in a normal manner. In cases where communications could not be accomplished in a normal manner is step S427, an error may be displayed in step S429, or a re-send command may be transmitted, and communications processing may be performed.

The communications processing in step S428 is completed; the processing then proceeds to step S416 in FIG. 63, the system controller 22 completes the alteration of the setting values of the peripheral device in question, and the operator confirms the results of the alterations on the concentrated display panel 20 or the like.

Furthermore, in the case of a protocol in which a request must be made for the updating of data, such as Bluetooth, wireless LAN or the like, a data updating request command may be transmitted from the PDA 68 is in step S423 in FIG. 64, and a determination as to whether the transmission and reception of data with the system controller 22 is possible may be made in step S424.

Furthermore, the system may also be devised so that the reception of vital sign data for the patient 48 from the abovementioned patient monitoring device 4 and functions such as the input of endoscopic images are also performed by the PDA 68 using the abovementioned operation.

The flow of the operation of the unidirectional infrared remote controller 69 will be described with reference to FIG. 48.

In step S431, the operator selects the UP/DOWN key in the area of the insufflator 14 shown in FIG. 13, and presses the command button. In step S432, infrared light is transmitted from the abovementioned infrared light output unit 184 of the unidirectional infrared remote controller 69. In step S433, the system controller 22 receives a key command transmitted by infrared light, and analyzes the reception data by the abovementioned filter processing and key command comparison. In step S434, the analyzed setting values of the insufflator 14 are altered.

The operation of the detection unit in FIGS. 61 and 62 will be described with reference to FIG. 65.

The wiring and the like of the endoscopic surgical system and the respective medical devices are set up as shown in FIG. 1. When preparations are completed, the icon of the PDA 68 is pressed, and the stored setting value information for the respective medical devices is read out and called up on the screen display part 165 of the PDA 68. Here, for example, a nurse confirms the procedure of the current endoscopic surgery, or setting values according to the doctor, and presses the transmission button of the transmission and reception buttons.

The setting value data for the respective medical surgery devices is transmitted to the system controller 22 from the PDA 68 by bidirectional infrared communications. Here, external light noise such as fluorescent lamps, natural light or the like is cut by an infrared-transmitting filter installed in the bidirectional infrared I/F 66 illustrated in FIG. 5.

Next, the flow of the operation in which the infrared light transmitted from the infrared-transmitting filter is processed inside the system controller 22 will be described with reference to FIG. 65.

In step S1001 in FIG. 65, the infrared signal transmitted from the PDA 68 is received by the light receiving element 1011 constructed in the bidirectional communications controller 1010 inside the infrared I/F 149 of the system controller 22, and this signal is converted into a current value that corresponds to the intensity of the received infrared light. In step S1002, this current value is converted into a voltage value by the I/V converter 1012.

In step S1003, the signal produced by conversion into a voltage is amplified to a signal of a predetermined level by the amplifier 1013, and only a predetermined frequency band of the infrared light is received by the BPF 1014. The gain corresponding to the attenuation of the infrared signal that depends on the distance between the PDA 68 and the system controller 22 is adjusted via the AGC 1015. In this way, a specified signal is received and subjected to waveform shaping.

The signal shaped in step S1004 is compared with a predetermined value by the detection unit 1016. The signal other than that of a specified level is discarded in step S1005, and only the predetermined signal from the PDA 68 is extracted in step S1006, thus producing the output signal.

In step S1007, the infrared control unit 1017 analyzes the predetermined output signal transmitted from the PDA 68, and transfers the data to the CPU 155a via the internal bus 154a.

On the basis of the transferred data, the CPU 155a alters the setting values for each medical device, so that preparations for endoscopic surgery are completed. In this way, infrared bidirectional communications are performed.

Next, for example, a case will be described in which the doctor operates the infrared remote controller 69 and receives a first infrared signal from the infrared remote controller 69 during surgery, while a second infrared signal is received at the same time from the PDA 68 from a nurse.

In this case, the propagation frequency bands in which the infrared light is propagated and the like are both similar; accordingly, both signals pass through the abovementioned infrared-transmitting filter or BPF.

As a result, as for the PDA 68, the desired control signal is received by the light receiving element on the side of the PDA 68 in steps S1001 through S1007, and waveform shaping is performed. In this case, the signal from the side of the infrared remote controller 69 is also mixed in.

Accordingly, the levels of the infrared light transmitted from the PDA 68 and infrared remote controller 69 are compared by the detection unit with the timing of step S1004.

For example, the level of the signal voltage shaped in the transmission data from the infrared remote controller 69 is assumed to be 4 V. Furthermore, the level of the signal voltage of the transmission data transmitted from the PDA 68 is assumed to be 5 V.

In the bidirectional infrared controller unit 1010, the input signals are compared by the detection unit 1017 with the reference that is preset by the infrared control unit 1019 set at 4.5 V.

In this case, it goes without saying that the set threshold value of the detection unit 1017 is close to 4.5 V with hysteresis.

Thus, only the signal data with a voltage of 5 V constituting the infrared signal from the PDA 68 is transmitted to the infrared control unit 1019, and the unnecessary signal is discarded by the detection unit 1017.

Furthermore, in the unidirectional infrared controller unit 1001, the input signals are conversely compared by the detection unit 1007 with the reference voltage that is preset by the infrared control unit 1009 set as described above at a value close to 4.5 V, and only the 4 V signal data is transmitted to the infrared control unit 1009.

FIG. 66 shows an actual infrared data waveform; for example, FIG. 66 shows the infrared data waveform in a case where an unnecessary signal is mixed in the blank period between the custom ID data specifying the device and the transmission data.

[Merits]

As a result of the abovementioned construction and effect, the control of desired medical devices can be accomplished without communications errors even when the infrared remote controller and PDA are used at the same time. Accordingly, the system is convenient to use, and the progress of surgery is not impeded.

A convenient system can be realized by providing remote controller that is suitable for respective settings made before and during surgery as described above.

[Seventh Embodiment]

Next, a seventh embodiment of the present invention will be described. A description of parts that are the same as in the sixth embodiment will be omitted. FIG. 67 is a block diagram which shows the essential parts of the construction of the PDA in this seventh embodiment of the present invention.

[Construction]

In the display part 165 of the PDA 68 shown in the abovementioned FIG. 57, the following construction is used: specifically, when the transmission button 355 is pressed in cases where the abovementioned comprehensive settings are performed, the setting values of the insufflator 14 and the like shown in the figure are transmitted to the system controller 22, and the communications processing state is displayed on a communications state display part 356.

[Effect]

When data is exchanged in the flow of transmission and reception in FIG. 64 described in the sixth embodiment, procedures such as communications establishment processing in step S423, transmission or reception of data in progress to or from the system controller 22 in step S425, data analysis in progress in step S426, communications completed in step S428 and the like must be used. Accordingly, the current processing state in data communications is displayed on the communications state display part 365.

Conceivable display contents include “communications being established”, “data reception (transmission) in progress”, “normal completion”, “communications error”, “insufflator mode unsatisfactory”, “insufflator in operation” and the like.

Furthermore, in cases where the abovementioned communications processing is performed at a high speed, the process may be displayed as an error log function indicating the stage at which an error has occurred when the data cannot be updated. In this case, the error log can be arranged as “communications establishment processing—pass→ID acquisition—pass→data transmission—fault”, and the operator can re-transmit while taking into account the content of the error log.

Furthermore, in cases where the setting value information transmitted to the system controller 22 is outside the range that can be set for the peripheral device in question, this may be displayed as a parameter setting range error on the PDA 68 or the like.

[Merits]

The present embodiment possesses the following merits: specifically, trouble that occurs when the operator makes a mistake can be quickly handled, the convenience of the remote controller device can be improved, and obstacles to the progress of surgery can be avoided.

[Eighth Embodiment]

An eighth embodiment of the present invention will be described. A description of parts that are the same as in the sixth and seventh embodiments will be omitted.

[Construction]

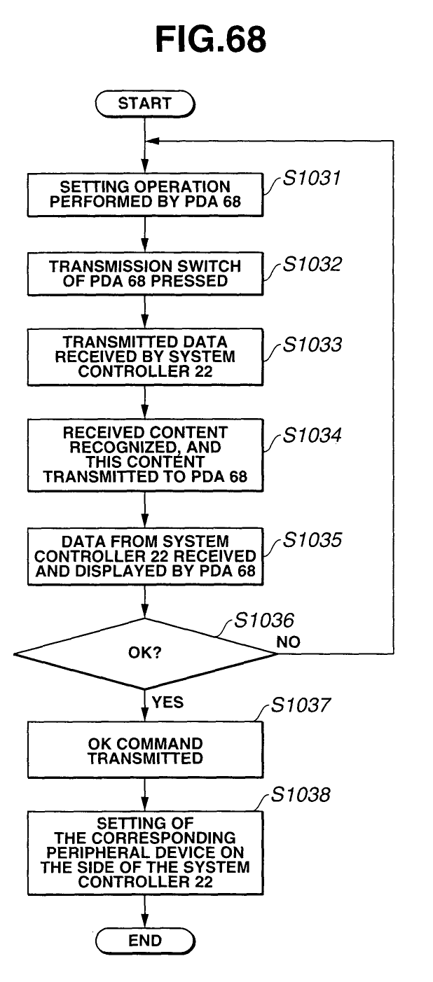

FIG. 68 shows a flow chart of the processing that takes place when the PDA 68 is operated.

[Effect]

Next, the flow chart shown in FIG. 68 will be described. In step S1031, the area of the insufflator 14 (see the insufflator setting value area in FIG. 56) of the PDA 68 is selected. In step S1032, infrared light is transmitted by pressing the command button for which a setting operation is desired. In step S1033, the system controller 22 receives the transmission data. In step S1034, the reception content is recognized, and the reception data is re-transmitted to the PDA 68. In step S1035, the PDA 68 receives the data and displays the content on the liquid crystal display part 165. In step S1036, the operator views the content, and if the operator confirms that this is the content that has been selected and transmitted by the operator himself, the operator presses the command button in step S1037, and transmits the data to the system controller 22. When the system controller 22 recognizes a notification of confirmation in step S1038, the setting values of the insufflator 14 are updated, and the processing is ended.

[Merits]

As a result of the abovementioned construction and effect, the following merits are obtained: for example, in the case of a conventional unidirectional infrared remote controller 69, the operator performs setting operations for peripheral devices by means of UP/DOWN commands, and can ensure safety by confirming the updated values on the display device 19. On the other hand, in cases where the PDA 68 is used, the received results can be sent back from the system controller 22 when the operator performs setting operations, so that the operator can be caused to re-confirm the values. Accordingly, a greater degree of safety can be maintained.

[Ninth Embodiment]

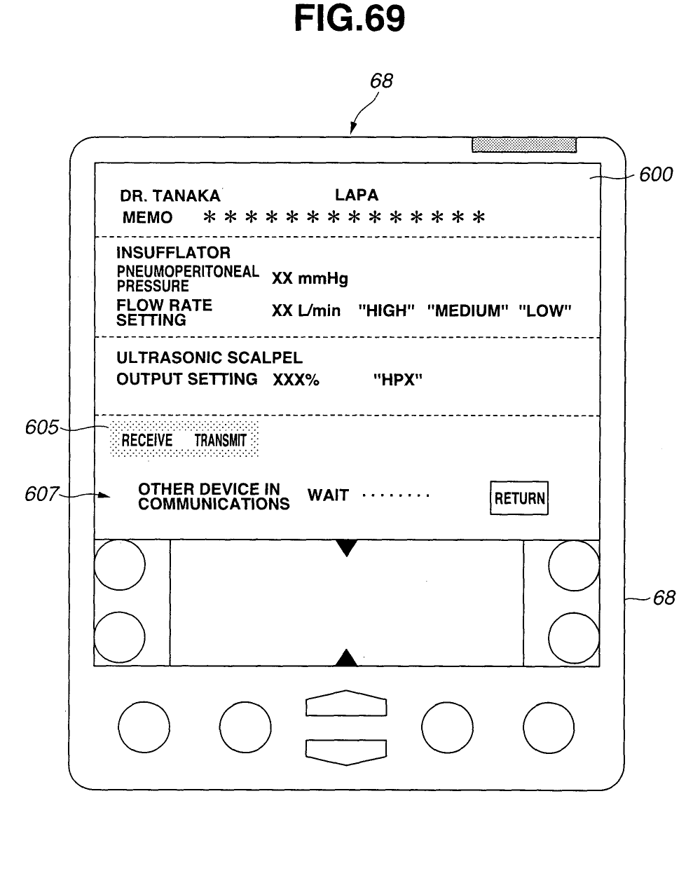

A ninth embodiment of the present invention will be described with reference to FIGS. 69 through 71.

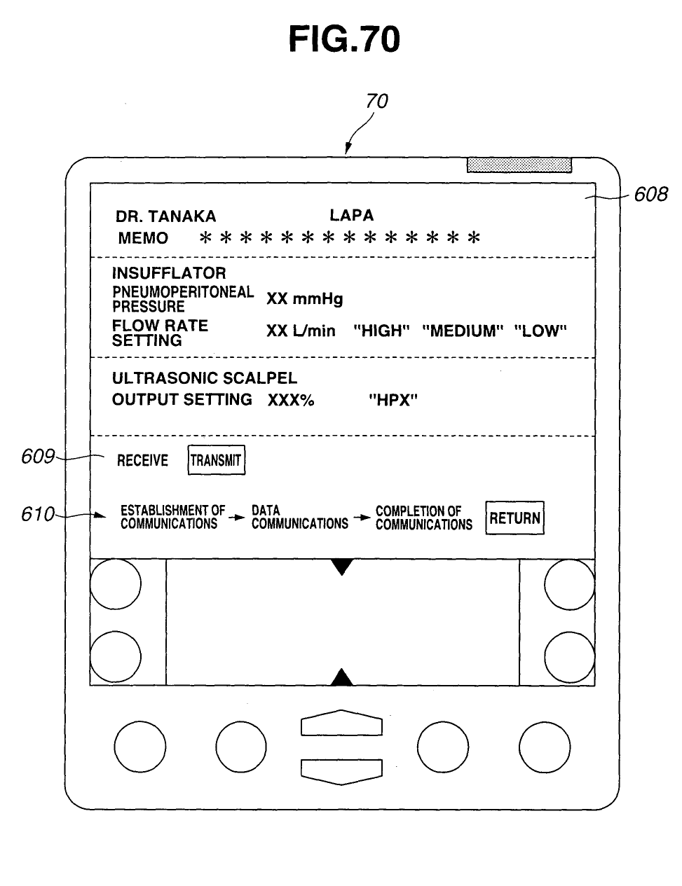

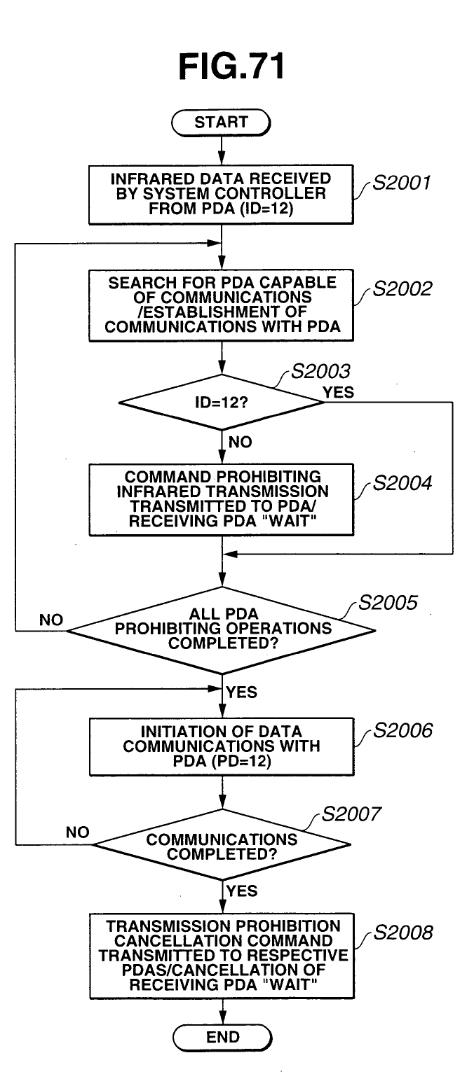

FIGS. 69 and 70 are diagrams which show the display states of the display parts of the PDA 68 and PDA 70 in a case where the system is constructed from a first PDA 68 and a second PDA 70, and remote controller operations are performed by the system controller 22 from one PDA 70. FIG. 71 is a flow which illustrates the software operation that transmits communications limiting commands from the system controller 22 to a specified PDA 68.

A description of constructions that are the same as in the sixth embodiment will be omitted.

[Construction]

FIG. 69 shows the first PDA 68; the first PDA 68 is constructed from a display part 600 which displays transmitted and received setting data for respective medical devices, a transmission and reception button 605 in which the commands are masked, and a communications status display part 607 which displays the state of communications between the PDA 68 and the system controller 22.

FIG. 70 shows the second PDA 70; the second PDA 70 is similarly constructed from a display part 608, a transmission and reception button 609 capable of command operations, and a communications status display part 610.

[Effect]

Here, in regard to the method used by the system controller 22 to distinguish between the respective PDAs, the system controller 22 can discriminate between individual IDs for each PDA in the abovementioned IrDA packet communications. Accordingly, the individual device IDs can be assigned as initial values each time that the software shown in FIG. 68 is downloaded from the system controller 22, or can be set for each PDA by an operation performed by the user.

Next, the operation of the present embodiment will be described with reference to FIG. 71.

In step S2001, the transmission and reception button 609 of the PDA 70 shown in FIG. 70 is pressed, and the CPU 155a of the system controller 22 receives infrared command data for infrared communications. The CPU 155a of the system controller 22 recognizes an individual ID number (ID=12) that distinguishes the PDA 70 from the received infrared data.

In step S2002, a search is made for PDAs other than ID=12, and communications are established. For example, it is assumed that the PDA 68 is in a range that allows communications with the system controller 22. As a result of the search made in step S2002, the system controller 22 establishes communications with the PDA 68. Then, if the ID is not ID=12 is step S2003, the system controller 22 transmits the data of a communications limiting command to the PDA 68, and proceeds to step S2005. When the ID is ID=12 in step S2003, the system controller 22 proceeds to step S2005.

When the PDA 68 receives a communications limiting command, infrared transmission operations are prohibited, and only infrared reception is enabled. In this case, a mask is applied to the transmission and reception button shown in FIG. 69, and a “communications impossible” display is performed by the communications status display part 7. In FIG. 69, this is displayed as “other device in communications wait”.

Next, in step S2005, if there is no PDA capable of communications other than the PDA 68, a determination is made that the infrared transmission operations of all PDAs other than the PDA 70 whose ID=12 have been prohibited.

Next, the processing proceeds to step S2006, and data communications are initiated with the PDA 70 whose ID=12. When transmission has been selected by the transmission and reception button 609 of the PDA 70 shown in FIG. 70, the communications status display part 610 displays a communications state transition.

When it is recognized in step S2007 that communications have been completed, the processing proceeds to step S2008, and the system controller 22 sends a communications prohibition cancellation command to the PDA 68 for which infrared communications had been prohibited. After the PDA 68 receives this transmission prohibition cancellation command, the PDA 68 enters a state in which infrared transmission is possible.

In the present embodiment, infrared communications are used; however, it would also be possible to apply this embodiment to wireless communications using electromagnetic waves.

[Merits]

As a result of the above effect, the following merits are obtained: specifically, infrared transmission and reception to the system controller from a plurality of PDAs can be prevented, so that communications can always be reliably performed with one PDA. Accordingly, an efficient remote controller operation can be accomplished, and obstacles to the progress of surgery can be prevented.

As was described above, the sixth through ninth embodiments possess the following merits: specifically, a remote controller operation that is free of communications errors can be performed in a control system using an infrared remote controller transmitted from unidirectional infrared communications and a PDA performing bidirectional infrared communications, so that the convenience of use can be improved.

Having described the preferred embodiments of the invention referring to the accompanying drawings, it should be understood that the present invention is not limited to those precise embodiments, and that various changes and modifications thereof could be made by one skilled in the art without departing from the spirit or scope of the invention as defined in the appended claims.

What is claimed is:

1. A medical control device comprising:
a first communications control unit which utilizes communications of a first protocol to transmit and receive data to and from a first medical device that is used to perform medical treatments;
a second communications control unit which utilizes communications of a second protocol that differs from the first protocol to transmit and receive data to and from a second medical device that is used to perform medical treatments; and
a control part which transmits and receives data utilizing communications of a third protocol that is shared by the first communications control unit and the second communications control unit, and which controls the first communications control unit and the second communications control unit.

2. The medical control device according to claim 1, wherein the control part has a memory part that stores priority information relating to the communications processing of the first communications control unit and the second communications control unit, and the first communications control unit and the second communications control unit are controlled on the basis of the priority information.

3. The medical control device according to claim 2, wherein the priority information is information that corresponds to the type of protocol.

4. The medical control device according to claim 2, wherein the priority information is information that corresponds to the type of medical device.

5. The medical control device according to claim 2, wherein the priority information is information that corresponds to the function of the medical device.

6. The medical control device according to claim 2, wherein the control part performs processing with the first communications control unit and second communications control unit split in a time series in accordance with the priority information.

7. A medical control device control method comprising:
a first communications control step in which the control of transmission and reception is accomplished using a first communications control circuit which transmits and receives data to and from a first medical device which is used to perform medical treatments utilizing communications of a first protocol;
a second communications control step in which the control of transmission and reception is accomplished using a second communications control circuit which transmits and receives data to and from a second medical device which is used to perform medical treatments utilizing communications of a second protocol that differs from the first protocol; and
a step in which control is performed using a control circuit which transmits and receives data utilizing communications of a third protocol that is shared by the first communications control circuit and the second communications control circuit, and which controls the first communications control circuit and the second communications control circuit.

8. The method according to claim 7, wherein the control circuit controls the first communications control circuit and the second communications control circuit on the basis of priority information relating to the communications processing of the first communications control circuit and the second communications control circuit.

9. The method according to claim 8, wherein the priority information is information that corresponds to the type of protocol.

10. The method according to claim 8, wherein the priority information is information that corresponds to the type of medical device.

11. The method according to claim 8, wherein the priority information is information that corresponds to the function of the medical device.

12. The method according to claim 8, wherein the control part performs processing with the first communications control unit and the second communications control unit split in a time series in accordance with the priority information.

13. A medical control device which controls a plurality of medical devices, and causes medical images to be displayed by display means, comprising:
information selection means for selecting control information for the medical devices and patient vital sign information; and
information superimposing means for causing the information selected by the information selection means to be dispersed and shown in a superimposed display by the display means.

14. The medical control device according to claim 13, wherein the information superimposing means display the selected information for a predetermined time.

15. A medical system device, comprising at least:
a control device that controls a plurality of medical devices; and
an operating panel that indicates the control content to the control device and displays the control status,
wherein data transmitting and receiving means that transmit and receive data by means of difference data are provided on the control device and operating panel, the data is successively transmitted to the control device by the operating panel, and the control device periodically loads the data that is successively transmitted from the operating panel.

16. The medical system device according to claim 15, further comprising detection means for periodically loading the data that is successively transmitted from the operating panel and detecting changes in this loaded data.

17. The medical system according to claim 16, wherein the detection means have a memory that stores initial data beforehand, and detect changes in the data by performing operations on the initial data stored in the memory and the loaded data.

18. A control system comprising:
a control device used for control;
first transmitting and receiving means disposed in the control device;
a plurality of remote controller means which respectively have second transmitting and receiving means that are capable of communicating operating information used to operate the control device with the first transmitting and receiving means;
remote controller discriminating means which are disposed in the control device, and which discriminate the remote controller means on the basis of communications information from the second transmitting and receiving means received by the first transmitting and receiving means;
first communications control means for controlling the first transmitting and receiving means so that regulation indicating information used to indicate transmission regulations is transmitted to the second transmitting and receiving means of remote controller means that differ from the remote controller means discriminated by the remote controller discriminating means; and
second communications control means which are provided in each of the plurality of remote controller means, and which control the second transmitting and receiving means on the basis of the regulation indicating information.

19. A control system comprising:
a control device used for control;
first remote controller means which have a first infrared transmitting part that can transmit operating information used to operate the control device;
an infrared receiving part which is installed in the control device in order to receive the operating information transmitted by the first infrared transmitting part;
second remote controller means which have a second infrared transmitting part capable of transmitting at a higher infrared intensity than the first infrared transmitting part; and
infrared signal separating means which are disposed in the control device, and which separate the infrared transmission signals received by the infrared receiving part using a predetermined threshold value.
